(19) United States
(12) Patent Application Publication
(10) Pub. No.: US 2024/0180895 A1
(43) Pub. Date: Jun. 6, 2024

(54) USE OF KAPPA OPIOID RECEPTOR AGONISTS TO TREAT CARDIOVASCULAR DISEASE

(71) Applicant: Board of Supervisors of Louisiana State University and Agricultural and Mechanical College, Baton Rouge, LA (US)

(72) Inventors: Jacob Meariman, River Ridge, LA (US); Daniel R. Kapusta, Slidell, LA (US); Juan Gao, New Orleans, LA (US)

(21) Appl. No.: 18/282,173

(22) PCT Filed: Mar. 21, 2022
(86) PCT No.: PCT/US2022/021165

§ 371 Date: Sep. 14, 2023

Related U.S. Application Data
(60) Provisional application No. 63/163,584, filed on Mar. 19, 2021.

(51) Int. Cl. A61K 31/485; A61K 31/4965; A61K 31/5415; A61K 31/635; A61K 38/07; A61P 9/12; A61P 13/12

(52) U.S. Cl. CPC A61K 31/485; A61K 31/4965; A61K 31/5415; A61K 31/635; A61K 38/07; A61P 9/12; A61P 13/12

(57) ABSTRACT

A method of method of preventing or reversing diuretic resistance, the method comprising administering to the subject an amount of a kappa opioid receptor agonist (KOA) sufficient to selectively increase urine output, wherein the KOA decreases or does not change urinary excretion of electrolytes.

This application claims priority from U.S. Provisional Application No. 63/163,584 filed on Mar. 19, 2021, the entire contents of which is hereby incorporated by referenced.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

A method of method of preventing or reversing diuretic resistance, the method comprising administering to the subject an amount of a kappa opioid receptor agonist (KOA) sufficient to selectively increase urine output, wherein the KOA decreases or does not change urinary excretion of electrolytes.

### BACKGROUND OF THE INVENTION

Diuretics are one of the most prescribed therapies throughout the world and have proven to be an effective first-line class of antihypertensive drugs. However, the thiazide and loop classes of diuretics are well known to cause many adverse effects, including severe and potentially dangerous electrolyte abnormalities.

### SUMMARY OF THE INVENTION

An aspect of the invention is directed to methods to reverse diuretic resistance. In one embodiment, the method comprises administering to the subject an amount of a kappa opioid receptor agonist (KOA) sufficient to selectively increase urine output. In another embodiment, the KOA decreases or does not change urinary excretion of electrolytes. In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

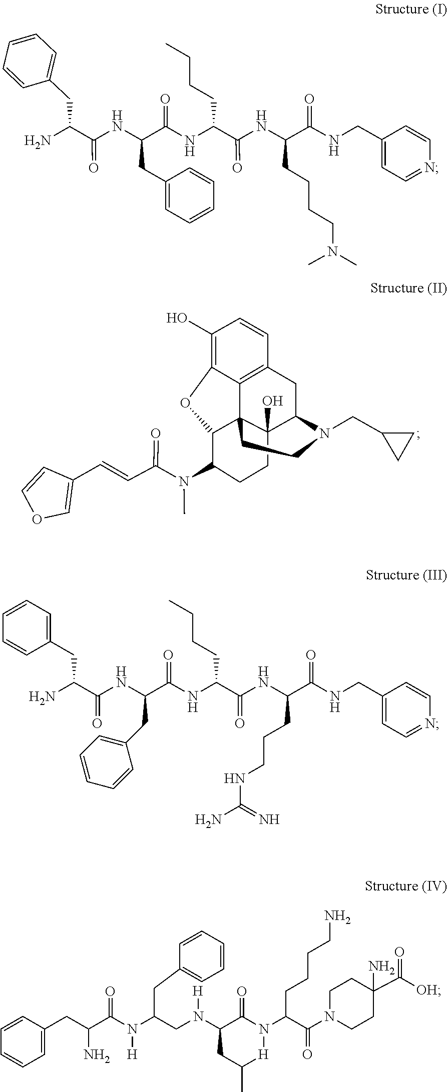

or any combination thereof. In some embodiments, the method further comprises administering to the subject one or more additional active agents. In yet further embodiments, the one or more additional active agents comprises a diuretic. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone.

An aspect of the invention is directed to methods of preventing diuretic resistance. In one embodiment, the method comprises administering to the subject an amount of a kappa opioid receptor agonist (KOA) sufficient to selectively increase urine output. In another embodiment, the KOA decreases or does not change urinary excretion of electrolytes. In a further embodiment, the KOA is administered in combination with a diuretic. In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

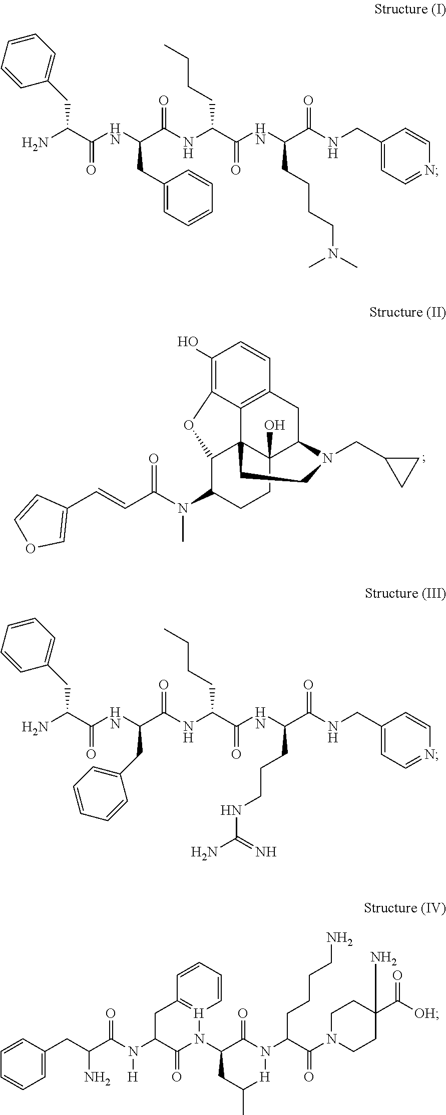

or any combination thereof. In some embodiments, the method further comprises administering to the subject one or more additional active agents. In yet further embodiments, the one or more additional active agents comprises a diuretic. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone.

An aspect of the invention is directed to methods of sensitizing a subject to a diuretic. In one embodiment, the method comprises administering to the subject an amount of a kappa opioid receptor agonist (KOA) sufficient to selectively increase urine output. In another embodiment, the KOA decreases or does not change urinary excretion of electrolytes. In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

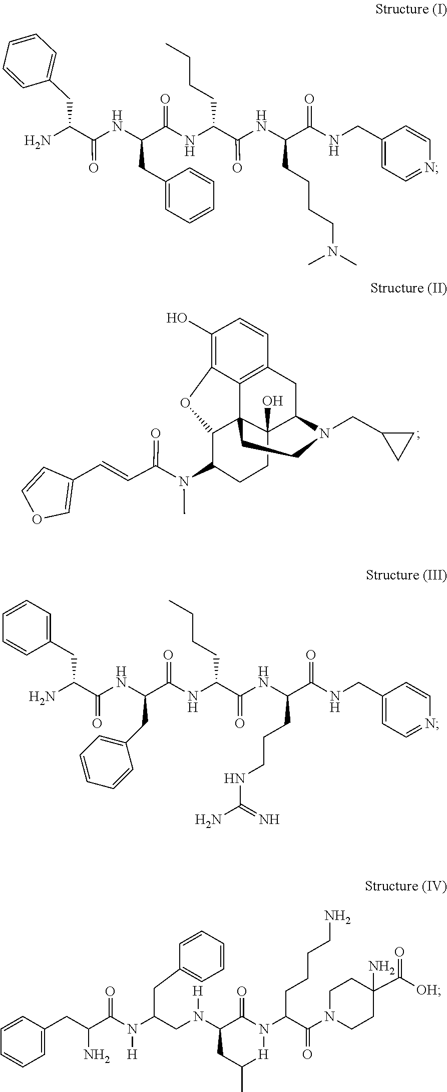

or any combination thereof. In some embodiments, the method further comprises administering to the subject one or more additional active agents. In yet further embodiments, the one or more additional active agents comprises a diuretic. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone.

An aspect of the invention is directed to methods of promoting urinary output. In one embodiment, the method comprises administering to the subject an amount of a kappa opioid receptor agonist (KOA) sufficient to selectively increase urine output. In another embodiment, the KOA reduces or does not change urinary excretion of electrolytes. In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

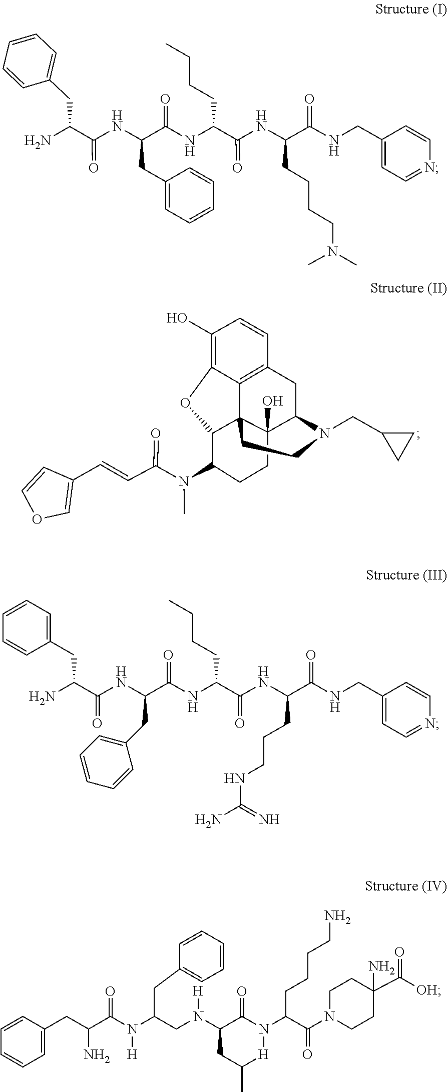

or any combination thereof. In some embodiments, the method further comprises administering to the subject one or more additional active agents. In yet further embodiments, the one or more additional active agents comprises a diuretic. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone.

An aspect of the invention is directed to methods of treating a subject afflicted with a cardiovascular disease. In some embodiments, the method comprises administering to the subject an amount of a kappa opioid receptor agonist (KOA) sufficient to selectively increase urine output. In another embodiment, the KOA decreases or does not change urinary excretion of electrolytes. In one embodiment, the treating comprises increasing urine output, decreasing urinary sodium and potassium excretion, decreasing urine osmolality, decreasing mean arterial pressure, or a combination thereof. In another embodiment, the cardiovascular disease comprises hypertension, heart failure, or a combination thereof. In a further embodiment, the kappa opioid receptor agonist decreases mean arterial pressure (MAP). In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

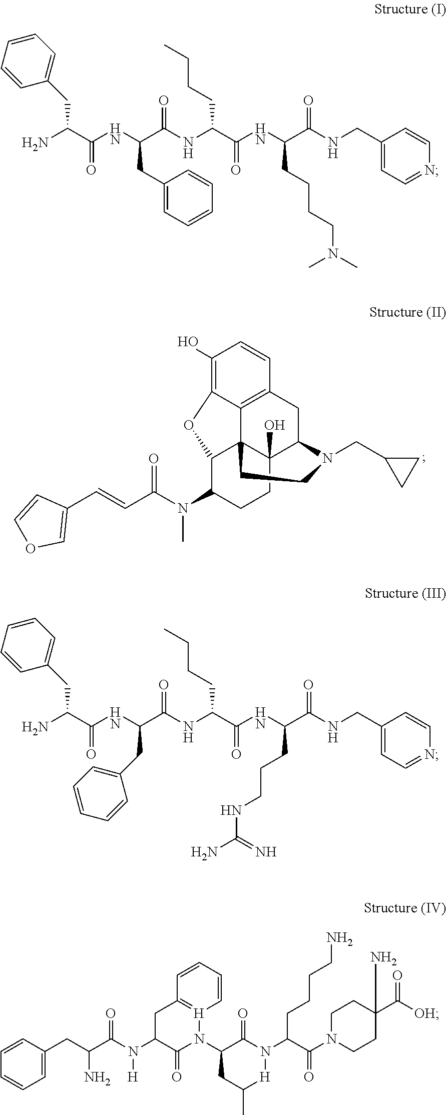

or any combination thereof. In some embodiments, the method further comprises administering to the subject one or more additional active agents. In yet further embodiments, the one or more additional active agents comprises a diuretic, an antihypertensive medication, or a combination of both agents. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone. In other embodiments, the antihypertensive medication comprises an angiotensin receptor blocking agent, an angiotensin converting enzyme inhibitor, a beta blocker, an alpha receptor blocker, a centrally acting sympatholytic, a calcium channel blocker, or a direct acting vascular smooth muscle dilator.

An aspect of the invention is directed to methods treating a subject afflicted with a renal disease. In some embodiments, the method comprises administering to the subject an amount of a kappa opioid receptor agonist (KOA) sufficient to selectively increase urine output. In another embodiment, the KOA decreases or does not change urinary excretion of electrolytes. In one embodiment, the treating comprises increasing urine output, decreasing urinary sodium and potassium excretion, decreasing urine osmolality, decreasing mean arterial pressure, or a combination thereof. In another embodiment, the renal disease comprises acute or chronic kidney disease. In some embodiments, the subject suffers from diuretic resistance. In other embodiments, the subject has failed to achieve diuresis with diuretic therapy prior to administering the KOA. In further embodiments, the kappa opioid receptor agonist (KOA) comprises an organic or synthetic small molecule, or a peptide. In other embodiments, the kappa opioid receptor agonist (KOA) is a peripherally restricted KOA, a centrally acting KOA, or both. In yet further embodiments, the electrolytes comprise sodium, potassium, chloride, or a combination of the electrolytes listed herein. In some embodiments, the organic small molecule or peptide is JT09, nalfurafine, CR665, or difelikefalin. In some embodiments, the organic or synthetic small molecule or peptide comprises a molecule of:

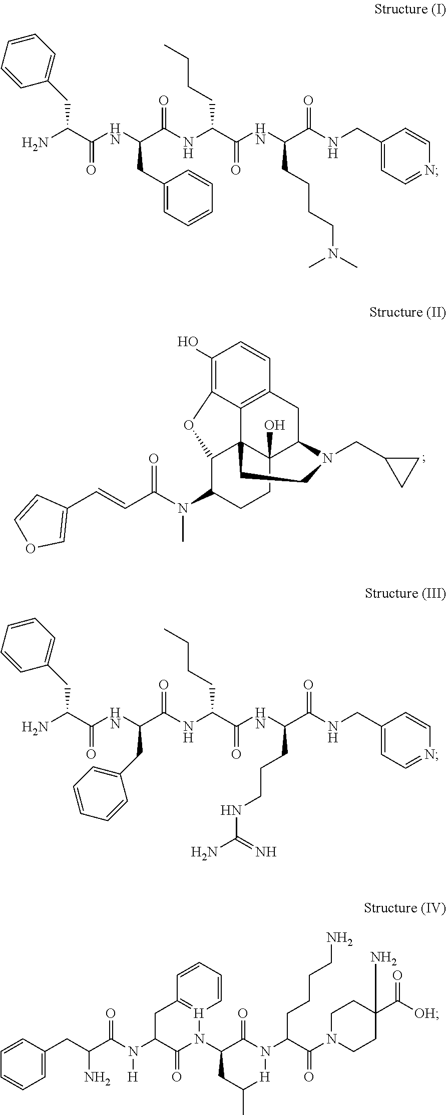

or any combination thereof. In some embodiments, the method further comprises administering to the subject one or more additional active agents. In yet further embodiments, the one or more additional active agents comprises a diuretic, an antihypertensive medication, or a combination of both agents. In other embodiments, the diuretic comprises a loop diuretic, a thiazide or thiazide-like diuretic, or a potassium sparing diuretic. In further embodiments, the loop diuretic comprises furosemide, torsemide, ethacrynic acid, or bumetanide. In yet further embodiments, the thiazide or thiazide-like diuretic comprises hydrochlorothiazide, chlorthalidone, metolazone, indapamide, chlorothiazide, or bendroflurnethiazide. In some embodiments, the potassium sparing diuretic comprises amiloride, triamterene, spironolactone, or eplerenone. In other embodiments, the antihypertensive medication comprises an angiotensin receptor blocking agent, an angiotensin converting enzyme inhibitor, a beta blocker, an alpha receptor blocker, a centrally acting sympatholytic, a calcium channel blocker, or a direct acting vascular smooth muscle dilator.

Other objects and advantages of this invention will become readily apparent from the ensuing description.

### BRIEF DESCRIPTION OF THE FIGURES

FIG. 1A and FIG. 1B show cardiovascular and renal excretory responses to i.v. administration of nalfurafine. Values are mean±SEM and demonstrate the cardiovascular and renal excretory responses produced by i.v. bolus administration of nalfurafine (5 μg kg−1; n=6) or vehicle (2 cc kg−1; n=6) in conscious Sprague-Dawley rats. FIG. 1A) Urine samples were collected for three consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; UNaV, urinary sodium excretion; UKV, urinary potassium excretion; HR, heart rate; MAP, mean arterial pressure. FIG. 1B) Total urine output, sodium concentration, potassium concentration, and urine osmolality were measured from the cumulative urine excreted during the 90-min protocol. *p<0.05 when compared between treatment groups; Student's t-test.

FIG. 2A and FIG. 2B show renal excretory responses to oral administration of nalfurafine. Values are mean±SEM and demonstrate the renal excretory response produced by oral administration of nalfurafine (150 μg kg−1; oral gavage) or vehicle (20 cc kg−1). Both groups are n=6, except where indicated for UNaV and UKV due to animals not producing urine at that time point. FIG. 2A) Urine samples were collected immediately after administration of drug/vehicle for 5 hours in consecutive 1-hour samples. ^{τ}p<0.05 when compared between treatment groups at respective time point; mixed linear model repeated measures, post hoc Sidak's. V, urine flow rate; UNaV, urinary sodium excretion; UKV, urinary potassium excretion. FIG. 2B) Total urine output, sodium concentration, and potassium concentration were measured from the cumulative urine excreted during the 5-hour protocol. *p<0.05 when compared between treatment groups; Student's t-test.

FIG. 3A and FIG. 3B show cardiovascular and renal excretory responses to i.v. administration of furosemide alone or in combination with nalfurafine. Values are mean±SEM and demonstrate the cardiovascular and renal excretory responses produced by i.v. bolus administration of furosemide alone (7.5 mg kg−1; n=6) or furosemide and nalfurafine (5 μg kg−1) (n=6). FIG. 3A) Urine samples were collected for three consecutive 10-min control periods and averaged for control value (C) and immediately after injection of drug for 90-min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; UNaV, urinary sodium excretion; UKV, urinary potassium excretion; HR, heart rate; MAP, mean arterial pressure. FIG. 3B) Total urine output, sodium concentration, potassium concentration, and urine osmolality were measured from the cumulative urine excreted during the 90-min protocol. *p<0.05 when compared between treatment groups; Student's t-test.

FIG. 4A and FIG. 4B show renal excretory responses to oral administration of furosemide alone or in combination with nalfurafine. Values are mean±SEM and demonstrate the renal excretory response to oral administration of furosemide alone (50 mg kg−1) or furosemide and nalfurafine (150 μg kg−1). Both groups are n=6, except where indicated for UNaV and UKV due to animals not producing urine at that time point. FIG. 4A) Urine samples were collected immediately after administration of drug for 5 hours in consecutive 1-hour samples. ^{τ}p<0.05 when compared between treatment groups at respective time point; mixed linear model repeated measures, post hoc Sidak's. V, urine flow rate; UNaV, urinary sodium excretion; UKV, urinary potassium excretion. FIG. 4B) Total urine output, sodium concentration, and potassium concentration were measured from the cumulative urine excreted during the 5-hour protocol. *p<0.05 when compared between treatment groups; Student's t-test.

FIG. 5A and FIG. 5B show cardiovascular and renal excretory responses to i.v. administration of HCTZ alone or in combination with nalfurafine. Values are mean±SEM and demonstrate the cardiovascular and renal excretory responses produced by i.v. bolus administration of HCTZ alone (2.5 mg kg−1; n=6) or HCTZ and nalfurafine (5 μg kg−1) (n=6). FIG. 5A) Urine samples were collected for three consecutive 10-min control periods and averaged for control value (C) and immediately after injection of drug for 90-min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; UNaV, urinary sodium excretion; UKV, urinary potassium excretion; HR, heart rate; MAP, mean arterial pressure. FIG. 5B) Total urine output, sodium concentration, potassium concentration, and urine osmolality were measured from the cumulative urine excreted during the 90-min protocol. *p<0.05 when compared between treatment groups; Student's t-test.

FIG. 6A and FIG. 6B show renal excretory responses to oral administration of HCTZ alone or in combination with nalfurafine. Values are mean±SEM and demonstrate the renal excretory response to oral administration of HCTZ alone (5 mg kg−1) or HCTZ and nalfurafine (150 μg kg−1). Both groups are n=6, except where indicated for UNaV and UKV due to animals not producing urine at that time point. FIG. 6A) Urine samples were collected immediately after administration of drug for 5 hours in consecutive 1-hour samples. ^{τ}p<0.05 when compared between treatment groups at respective time point; mixed linear model repeated measures, post hoc Sidak's. V, urine flow rate; UNaV, urinary sodium excretion; UKV, urinary potassium excretion. FIG. 6B) Total urine output, sodium concentration, and potassium concentration were measured from the cumulative urine excreted during the 5-hour protocol. *p<0.05 when compared between treatment groups; Student's t-test.

FIG. 7A and FIG. 7B show cardiovascular and renal excretory responses to i.v. administration of amiloride alone or in combination with nalfurafine. Values are mean±SEM and demonstrate the cardiovascular and renal excretory responses to i.v. bolus administration of amiloride alone (2 mg kg−1; n=6) or amiloride and nalfurafine (5 μg kg−1) (n=6). FIG. 7A) Urine samples were collected for three consecutive 10-min control periods and averaged for control value (C) and immediately after injection of drug for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; UNaV, urinary sodium excretion; UKV, urinary potassium excretion; HR, heart rate; MAP, mean arterial pressure. FIG. 7B) Total urine output, sodium concentration, and potassium concentration were measured from the cumulative urine excreted during the 90-min protocol. *p<0.05 when compared between treatment groups; Student's t-test.

FIG. 8A and FIG. 8B show renal excretory responses to oral administration of amiloride alone or in combination with nalfurafine. Values are mean±SEM and demonstrate the renal excretory response to oral administration of amiloride alone (5 mg kg−1) or amiloride and nalfurafine (150 μg kg−1). Both groups are n=6, except where indicated for UNaV and UKV due to animals not producing urine at that time point. FIG. 8A) Urine samples were collected immediately after administration of drug for 5 hours in consecutive 1-hour samples. ^{τ}p<0.05 when compared between treatment groups at respective time point; mixed linear model repeated measures, post hoc Sidak's. V, urine flow rate; UNaV, urinary sodium excretion; UKV, urinary potassium excretion. FIG. 8B) Total urine output, sodium concentration, potassium concentration, and urine osmolality were measured from the cumulative urine excreted during the 5-hour protocol. *p<0.05 when compared between treatment groups; Student's t-test.

FIG. 9A and FIG. 9B show cardiovascular and renal excretory responses to intravenous administration of difelikefalin. Values are mean±SEM and demonstrate the cardiovascular and renal excretory responses produced by intravenous administration of difelikefalin (10 μg/kg; n=6) or vehicle (2 cc/kg; n=6) in conscious Sprague-Dawley rats. FIG. 9A) Urine samples were collected for two consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; UNaV, urinary sodium excretion; U_{K}V, urinary potassium excretion; HR, heart rate; MAP, mean arterial pressure. FIG. 9B) Total urine output, sodium concentration, potassium concentration, and urine osmolality were measured from the cumulative urine excreted during the 90-min protocol. *p<0.05 when compared between treatment groups; Student'st-test.

FIG. 10A and FIG. 10B show cardiovascular and renal excretory responses to intravenous administration of difelikefalin following ICV norBNI pretreatment. Values are mean±SEM and demonstrate the cardiovascular and renal excretory responses produced by intravenous administration of difelikefalin (10 μg/kg) following pretreatment with ICV norBNI (1 μg/5 μL; n=6) or ICV vehicle (5 μL; n=6) in conscious Sprague-Dawley rats. FIG. 10A) Urine samples were collected for two consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; U_{Na}V, urinary sodium excretion; U_{K}V, urinary potassium excretion; HR, heart rate; MAP, mean arterial pressure. FIG. 10B) Total urine output, sodium concentration, potassium concentration, and urine osmolality were measured from the cumulative urine excreted during the 90-min protocol. *p<0.05 when compared between treatment groups; Student's t-test.

FIG. 11A and FIG. 11B show cardiovascular and renal excretory responses to intravenous administration of nalfurafine following ICV norBNI pretreatment. Values are mean±SEM and demonstrate the cardiovascular and renal excretory responses produced by intravenous administration of nalfurafine (5 μg/kg) following pretreatment with ICV norBNI (1 μg/5 μL; n=6) or ICV vehicle (5 μL; n=6) in conscious Sprague-Dawley rats. FIG. 11A) Urine samples were collected for two consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; U_{Na}V, urinary sodium excretion; U_{K}V, urinary potassium excretion; HR, heart rate; MAP, mean arterial pressure. FIG. 11B) Total urine output, sodium concentration, potassium concentration, and urine osmolality were measured from the cumulative urine excreted during the 90-min protocol. *p<0.05 when compared between treatment groups; Student's t-test.

FIG. 12A and FIG. 12B show cardiovascular and renal excretory responses to intravenous administration of difelikefalin following IV norBNI pretreatment. Values are mean±SEM and demonstrate the cardiovascular and renal excretory responses produced by intravenous administration of difelikefalin (10 μg/kg) following pretreatment with IV norBNI (30 μg/kg; n=6) or IV vehicle (2 cc/kg; n=6) in conscious Sprague-Dawley rats. FIG. 12A) Urine samples were collected for two consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; U_{Na}V, urinary sodium excretion; U_{K}V, urinary potassium excretion; HR, heart rate; MAP, mean arterial pressure. FIG. 12B) Total urine output, sodium concentration, potassium concentration, and urine osmolality were measured from the cumulative urine excreted during the 90-min protocol. *p<0.05 when compared between treatment groups; Student's t-test.

FIG. 13A and FIG. 13B show cardiovascular and renal excretory responses to intravenous administration of nalfurafine following IV norBNI pretreatment. Values are mean±SEM and demonstrate the cardiovascular and renal excretory responses produced by intravenous administration of nalfurafine (5 μg/kg) following pretreatment with IV norBNI (30 μg/kg; n=6) or IV vehicle (2 cc/kg; n=6) in conscious Sprague-Dawley rats. A) Urine samples were collected for two consecutive 10-min control periods and averaged for control value (C) and immediately after injection of drug/vehicle for 90 min in consecutive 10-min samples. *p<0.05 when compared to respective group control value C; two-way repeated measures ANOVA, post hoc Dunnett's. ^{τ}p<0.05 when compared between treatment groups at respective time point; two-way repeated measures ANOVA, post hoc Sidak's. V, urine flow rate; U_{Na}V, urinary sodium excretion; U_{K}V, urinary potassium excretion; HR, heart rate; MAP, mean arterial pressure. B) Total urine output, sodium concentration, potassium concentration, and urine osmolality were measured from the cumulative urine excreted during the 90-min protocol. *p<0.05 when compared between treatment groups; Student's t-test.

FIG. 14 shows difelikefalin prevents diuretic resistance to repeated daily administration of furosemide. Values are mean±SEM and demonstrate the 24-hour renal excretory responses produced by once daily intraperitoneal (IP) administration of furosemide alone (10 mg/kg; n=8) or the combination of furosemide and difelikefalin (20 μg/kg; n=6). Consecutive 24-hour urine samples were collected after administration of drug in Sprague-Dawley rats. τ P<0.05 when compared between treatment groups at respective time point and *P<0.05 interaction between treatment groups; 2-way repeated measures ANOVA, post hoc Sidak. UNaV, urinary sodium excretion; UKV, urinary potassium excretion.

FIG. 15 shows nalfurafine prevents diuretic resistance to repeated administration of furosemide. Values are mean±SEM and demonstrate the 5-hour renal excretory responses produced by twice daily intraperitoneal (IP) administration of furosemide alone (20 mg/kg; n=6) or the combination of furosemide and nalfurafine (10 μg/kg; n=6). Urine samples were collected daily for 5-hours after the first administration of drug. τ P<0.05 when compared between treatment groups at respective time point and *P<0.05 interaction between treatment groups; 2-way repeated measures ANOVA, post hoc Sidak. UNaV, urinary sodium excretion; UKV, urinary potassium excretion.

FIG. 16 shows difelikefalin rescues diuretic resistance caused by repeated administration of furosemide in rats consuming a low sodium diet. Values are mean±SEM and demonstrate the 5-hour renal excretory responses produced by once daily intraperitoneal (IP) administration of furosemide alone (10 mg/kg; n=6; days 1-12; open circles) and furosemide in combination with difelikefalin (20 μg/kg; days 13-17; filled circles) in rats placed on a low sodium diet. UNaV, urinary sodium excretion; UKV, urinary potassium excretion.

FIG. 17 shows nalfurafine rescues diuretic resistance following repeated daily administration of furosemide. Values are mean±SEM and demonstrate the 5-hour renal excretory responses produced by twice daily oral administration of furosemide alone (50 mg/kg; n=3). Nalfurafine (150 μg/kg) was added to furosemide treatment on day 7. Urine samples were collected 5 hours after administration of drug. UNaV, urinary sodium excretion; UKV, urinary potassium excretion.

FIG. 18 shows changes in blood pressure produced by chronic oral administration of nalfurafine. Values are mean±SEM and demonstrate the changes in blood pressure produced by oral administration of nalfurafine (0.5 mg/kg/day; n=8) or vehicle (n=8) in conscious spontaneously hypertensive rats over 5 weeks. τ p<0.05 significant interaction between treatment groups over time; two-way repeated measures ANOVA. *p<0.05 when compared between treatment groups at respective time point; post hoc Sidak's.

FIG. 19 shows cChanges in blood pressure produced by chronic subcutaneous infusion of nalfurafine. Values are mean±SEM and demonstrate the changes in blood pressure produced by oral administration of nalfurafine (0.3 mg/day; n=7) or vehicle (n=6) in conscious spontaneously hypertensive rats over 5 weeks. τ p<0.05 significant interaction between treatment groups over time; two-way repeated measures ANOVA. *p<0.05 when compared between treatment groups at respective time point; post hoc Sidak's.

### DETAILED DESCRIPTION OF THE INVENTION

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Administration of a diuretic to animals or man causes an increase in urine output (diuresis) by acting within the kidneys to prevent the renal tubular reabsorption of sodium, which over time can lead to diuretic-induced hyponatremia, hypokalemia, and activation of compensatory mechanisms (e.g., increased renal tubular sodium reabsorption in nephron segments not blocked by the diuretic, central release of antidiuretic hormone) which lead to loss of diuretic efficacy (diuretic resistance).

Administration of a kappa opioid receptor agonist (KOA) to animals or man causes an increase in urine output (diuresis) associated with either no change or a decrease in urinary electrolyte excretion (e.g., sodium, potassium, chloride); this pattern of changes in water and electrolytes is referred to as a water diuresis (aquaresis). See, for example, Horwell et al., U.S. Pat. Nos. 4,663,343, 4,906,655, 4,965,278, 5,019,588, 5,063,242; Clemence et al., U.S. Pat. Nos. 4,888,355, 4,988,727; Zimmerman et al., U.S. Pat. Nos. 4,891,379, 4,992,450, 5,064,834, 5,319,087, 5,422,356; Naylor et al., U.S. Pat. No. 5,116,842; Moura et al., U.S. Pat. Nos. 5,068,244, 5,130,329; and Mcknight et al., U.S. Pat. Nos. 5,317,028, and 5,369,105.

Aspects of the invention are drawn to new uses of compositions comprising kappa opioid receptor agonists. For example, the uses comprise methods of preventing and/or reversing diuretic resistance, the method comprising administering to the subject an amount of a kappa opioid receptor agonist (KOA) sufficient to selectively increase urine output, wherein the KOA decreases or does not change urinary excretion of electrolytes.

Endogenous opioid receptors have been identified in both the central nervous system (brain and spinal cord), and in the periphery. These receptors have been classified into three major subtypes: mu, delta, and kappa receptors.

The kappa opioid receptor (KOR) is a G protein-coupled receptor that in humans is encoded by the OPRKI gene. KORs are widely distributed in the brain, spinal cord, and in peripheral tissues. Based on receptor binding studies, three variants of the KOR referred to as K1, K2, and K3 have been characterized.

The so-called “kappa opioid agonists,” bind to kappa receptors with high selectivity. A compound is considered a kappa opioid agonist if it binds to kappa receptors in a binding assay, or if it demonstrates kappa agonist activity in functional assays.

The term “agonist” can refer to a compound that binds to a receptor, such as the KOR, and elicits a response in the cell. An “agonist” can mimic the action of an endogenous ligand, such as a hormone, peptide, or protein, and causes a physiological response similar to that produced by an endogenous ligand.

The term “partial agonist” can refer to a compound that binds to a receptor and induces a partial response in a cell. For example, a partial agonist can produce only a partial physiological response of the endogenous ligand. For example, the response is about 10%, about 20%, about 30%, about 40%, about 50%, about 60%, about 70%, about 80%, about 90%, or about 99% of the full physiological response of an endogenous ligand.

For example, a kappa opioid receptor agonist can refer to a compound that binds to and activates a specific kappa opioid receptor type. Any pharmacologically acceptable kappa opioid agonist will function in the invention, as the underlying mechanism is dependent upon binding to and stimulating kappa opioid receptors.

Categories of kappa opioid agonists include (1) the dynorphins, which are endogenous peptides and their derivatives; (2) the benzodiazepine derivatives, such as tifluadom; (3) the benzomorphan derivatives, such as ethylketocyclazocine, ketocyclazocine, and bremazocine; (4) the benzeneacetamide derivatives, such as U-50,488H, U-62,066E, U-69,593, CI-977, and PD 117302; and (5) the aminomethylpyridines, such as BRL 52537, BRL 52656, BRL 53114, GR89696, GR86014, and GR91272. Kappa opioid agonists can also include short chain peptides. See, for example, U.S. Pat. No. 7,402,564. For example, JT09 and difelikefalin are each peptides.

Non-limiting examples of kappa opioid agonists are listed herein. See, for example, U.S. Pat. No. 5,859,043, which is incorporate by reference herein in its entirety. In many cases the listings include references to a commercial source, a citation for the synthesis of a compound, or both.

I. L Kappa Opioid Agonists available through SmithKline Beecham Pharmaceuticals, Department of Renal Pharmacology, King of Prussia, Pa.; or Department of Biology, SmithKline Beecham Farmaceutici, Baranzate, Milan, Italy:
BRL 52537 (2S)-1-[3,4-dichlorophenylacetyl]-2-[(1-pyrrolidinylmethyl]-piperidine
BRL 52656 (2S)-1-[4-trifluoromethyl-phenyl]acetyl]-2-[(1-pyrrolidinyl)methyl]piperidine V. Vecchietti et al., J. Med. Chem., vol. 34, pp. 397-403 (1991).
BRL 53114 (−)-1-(4-trifluoromethylphenyl)-acetyl-2-(1-pyrrolidinylmethyl)-3,3-dimethyl -piperidine hydrochloride
BRL 52974 4-(1-pyrrolidinylmethyl)-5-(3,4-dichlorophenyl)acetyl-4,5,6,7-tetrahydroimidazo-[4,5-c]-pyridine
BRL 53117 1-[(3,4-dichlorophenyl)acetyl]-2-[(3-hydroxy-1-pyrrolidinyl)-methyl]-4,4-dimethylpiperidine
BRL 52974 5-[(3,4-dichlorophenyl)acetyl]-4-(1-pyrrolidinylmethyl)-4,5,6,7-tetrahydro -1H-imidazo[4,5-c]pyridine
BRL 53001 (2S)-2-(dimethylaminomethyl)-1-[(5,6,7,8-tetrahydro-5-oxo-2-naphthyl)acetyl]piperidine
2-(aminomethyl)piperidine derivatives, with incorporation of the 1-tetralon-6-yl-acetyl residue: G. Giardina et al., J. Med. Chem., vol. 37, pp. 3482-3491 (1994)
Compound (34) (2S)-2-[(dimethylamino)-methyl]-1-[(5,6,7,8-tetrahydro-5-oxo-2-naphthyl)-acetyl]-piperidine

II. Kappa Opioid Agonists available through The Upjohn Company, Kalamazoo, Mich.:
U-50,488H trans-(+/−)-3,4-dichloro-N-methyl-N-[2-(1-pyrrolidinyl)cyclohexyl]-benzeneacetamide methanesulphonate hydrate
J. Szmuszkovicz et al., J. Med. Chem., vol. 25, pp. 1125-1126 (1982); U.S. Pat. Nos. 4,098,904; 4,145,435; European Pat. No. 0 129 991 (1985); Chem. Abstr. vol. 91, no. 39003g (1979).
U-62,066E (spiradoline) 3,4-dichloro-N-methyl-N-(3-methylene-2-oxo-8-(1-pyrrolidinyl) -1-oxaspiro(4,5)dec-7-yl)-benzeneacetamide
J. Szmuszkovicz et al., J. Med. Chem., vol. 25, pp. 1125-1126 (1982); U.S. Pat. No. 4,438,130; Chem Abstr. vol. 101, no. 54912w; German Pat. No. 3241933 (1985); Chem. Abstr. vol. 103, no. 184969b (1985).
U-69,593 (5-α,7-α,8-β)-(−)-N-methyl-N-[7-(1-pyrrolidinyl)-1-oxaspiro(4,5)-dec-8-yl]benzeneacetamide
R. A. Lahti et al., Eur. J. Pharmacol., vol. 109, pp. 281-284 (1985).

III. Kappa Opioid Agonists available through Parke-Davis Research Unit, Addenbrooke's Hospital Site, Hills Road, Cambridge, England, or Parke-Davis Research Division, Warner-Lambert Company, Ann Arbor, Mich.
CI-977 (enadoline=PD 129290) (5R)-(5-α, 7-α, 8-β)-N-methyl-N-[7-(1-pyrrolidinyl)-1-oxaspiro[4,5]dec-8-yl]-4-benzo-furanacetamide monohydrochloride
P. R. Halfpenny et al., J. Med. Chem., vol. 33, pp. 286-291 (1990); P. R. Halfpenny et al., J. Med. Chem., vol. 34, pp. 190-194 (1991).
PD 117302 (+/−)-trans-N-methyl-N-[2-(1-pyrrolidinyl)-cyclohexyl]-benzo[b]-thiophene-4-acetamide monohydrochloride
C. R. Clark et al., J. Med. Chem., vol. 31, pp. 831-836 (1988).
Derivatives of PD 117302:
A. Compound (9) (+)-N-methyl-N-[7-(1-pyrrolidinyl)-1-oxaspiro[4.5]dec-8-yl]acenaphthene -carboxamide monohydrochloride
P. R. Halfpenny et al., J. Med. Chem., vol. 34, pp. 190-194 (1989).
B. Compound (17) (−)-4,5-dihydro-N-methyl-N-[7-(1-pyrrolidinyl)-1-oxaspiro[4.5]dec-8-yl]-3H-naphtho-[1,8-b,c]-thiophene-5-carboxamide-p-toluene sulfonate
P. R. Halfpenny et al., J. Med. Chem., vol. 34, pp. 190-194 (1989).
C. Compound (32) trans-(+/−)-N-methyl-N-[4,5-dimethoxy-2-(1-pyrrolidinyl)-cyclohexyl]-benzo[b]-thiophene-4-acetamide
P. R. Halfpenny et al., J. Med. Chem., vol. 32, pp. 1620-1626 (1989).
D. Compound (21) (−)-(5-β, 7-β, 8-α)-N-methyl-N-7-(1-pyrrolidinyl)-1-oxaspiro [4.5]-dec-8-yl]-benzo[b]furan-4-acetamide monohydrochloride
P. R. Halfpenny et al., J. Med. Chem., vol. 33, pp. 286-291 (1990).

IV. Kappa Opioid Agonists available through Zambeletti Research Laboratories, Baranzate, Milan, Italy
(2S)-1-(arylacetyl)-2-(aminomethyl)piperidine derivatives:
V. Vecchietti et al., J. Med. Chem., vol. 34, pp. 397-403 (1991); V. Vecchietti et al., J. Med. Chem., vol. 34, pp. 2624-2633 (1991).
A. Compound (14) (=BRL 52537A) (2S)-1-[3,4-dichlorophenyl-acetyl]-2-(pyrrolidin-1-yl -methyl) piperidine hydrochloride
B. Compound (21) (=BRL 52656A) (2S)-1-[[4-(trifluoromethyl)phenyl]acetyl]-2-(pyrrolidin -1-yl-methyl) piperidine hydrochloride (1S)-1-(aminomethyl)-2-(arylacetyl)-1,2,3,4-tetrahydroisoquinoline and heterocycle -condensed tetrahydropyridine derivatives:
V. Vecchietti et al., J. Med. Chem., vol. 34, pp. 2624-2633 (1991).
A. Compound (28)
B. Compound (30)
C. Compound (48)

V. Kappa Opioid Agonists available through The Du Pont Merck Pharmaceutical Co., Wilmington, Del.
DuP747 3,4-dichloro-N-methyl-N-(2-(pyrrolidin-1-yl)-1,2,3,4-tetrahydro-5-hydroxynaphthalen-1-yl)-benzeneacetamide
M. A. Hussain et al., Pharm. Res., vol. 9, pp. 750-752 (1992).
DuP E3800 (+/−)-trans-3,4-dichloro-N-methyl-[2-(pyrrolidine-1-yl)-6-hydroxy -1,2,3,4tetrahydronaphth-1-yl]-benzeneacetamide phosphate

VI. Kappa Opioid Agonists available through Preclinical Pharmaceutical Research, and Department of Medicinal Chemistry, E. Merck, Darmstadt, Germany; or Merck-Clevenot S. A., Nogent-sur-Marne, France
EMD 60400 N-methyl-N-[(1S)-1-phenyl-2-((3S)-3-hydroxypyrrolidine-1-yl)-ethyl]-2-aminophenylacetamide 2HCl
A. Barber et al., Br. J. Pharmacol., vol. 111, pp. 843-851 (1994).
EMD 61753 R. Gottschlich et al., Chirality, vol. 6, pp. 685-689 (1994); A. Barber et al., Br. J. Pharmacol., vol. 113, pp. 1317-27 (1994).

VII. Kappa Opioid Agonists available through Glaxo Group Research Ltd., Dept. of Medicinal Chemistry and Neuropharmacology, Ware, Herefordshire, England:
1-[(3,4-dichlorophenyl)acetyl]-2-[(alkylamino)methyl]piperidine derivatives:
D. Scopes et al., J. Med. Chem., vol. 35, pp. 490-501 (1992); A. Hayes et al., Br. J. Pharmacol., vol. 101, pp. 944-948 (1990); H. Rogers et al., Br. J. Pharmacol., vol. 106, pp. 783-789 (1992).
A. Compound (10) 1-[3,4-dichlorophenyl-acetyl]-2-[1-(3-oxopyrrolidinyl)]methyl]piperidine
B. Compound (39) (-GR 45809) 8-[3,4-dichlorophenyl-acetyl]-7-(1-pyrrolidinylmethyl)-1,4-dioxa-8-azaspiro[4,5]decane
C. GR89696 methyl-4-[3,4-dichlorophenyl-acetyl]-3-(1-pyrrolidinylmethyl)-1-piperazinecarboxylate fumarate
D. GR86014 2-[(3,4-dichlorophenyl)acetyl]-1,2,3,4-tetrahydro-1-(1-pyrrolidinyl-methyl) -Sisoquinolinol maleate
E. GR91272 5-[(3,4-dichlorophenyl)-acetyl]-4,5,6,7-tetrahydro-4-[(3-hydroxy-1-pyrrolidinyl)-methyl]furo 3,2-c]pyridine hydrochloride
F. GR44821 1-[(3,4-dichlorophenyl)acetyl]-2-[(3-oxo-1-pyrrolidinyl)methyl]piperidine maleate
G. GR103545 (R)-methyl-4-[(3,4-dichlorphenyl)acetyl]-3-(1-pyrrolidinyl-methyl)-1-piperazinecarboxylate fumarate
H. GR94839
I. GR85571
5-(arylacetyl)-4-[(alkylamino)methyl]furo 3,2-c]pyridines:
A. Naylor et al., J. Med. Chem., vol. 37, pp. 2138-44 (1994).
A. Compounds (16-23)
B. Compound (26) (=GR107537)
C. Compound (27)
Substituted trans-3-(decahydro- and octahydro-4a-isoquinolinyl) phenols:
D. Judd et al., J. Med. Chem., vol. 35, pp. 48-56 (1992).
A. Compound (10)
B. Compounds (11 a-d)
C. Compound (20)

VIII. Kappa Opioid Agonists available through ICI Pharmaceutical, Research Department, Alderley Park, Macclesfield, Cheshire, England
G. Costello et al., Eur. J. Pharmacol, vol. 151, pp. 475-478 (1988).
ICI 204879 (R,S)-N-[2-(N-methyl-3,4-dichlorophenylacetamido)-2-(3,4-dimethyloxyphenyl)-ethyl]pyrrolidine hydrochloride
ICI 199441 (2S)-N-[2-(N-methyl-3,4-dichlorophenylacetamido)-2-phenylethyl]pyrrolidine hydrochloride
ICI 197067 (2S)-N-[2-(N-methyl-3,4-dichlorophenylacetamido)-3-methylbutyl]pyrrolidine hydrochloride
2-(3,4-dichlorophenyl)-N-[2-(1-pyrrolidinyl)ethyl] acetamide derivatives (U-50,488 derivatives):
G. Costello et al., J. Med. Chem., vol. 34, pp. 181-189 (1991).
A. Compound (8) 2-(3,4-dichlorophenyl)-N-methyl-N-[(1S)-1-phenyl-2-(1-pyrrolidinyl)ethyl]acetamide
2-(3,4dichlorophenyl)-N-methyl-N-[2-(1-pyrrolidinyl)-1-substituted-ethyl]acetamides:
J. Barlow et al., J. Med. Chem., vol. 34, pp. 3149-3158 (1991).
A. Compound (13) 2-(3,4-dichlorophenyl)-N-methyl-N-[(1S)-1-(1-methylethyl)-2-(1-pyrrolidinyl)-ethyl]acetamide
B. Compound (48) 2-(3,4-dichlorophenyl)-N-methyl-N-[(1R,S)-1-(3-aminophenyl)-2-(1-pyrrolidinyl)-ethyl]acetamide

IX. Kappa Opioid Agonists available through Medizinische Klinik II and Institut fur Klinische Chemie, Klinikum Grosshadern, Munchen, Germany; or Humanpharmakologisches Zentrum, Boehringer Ingelheim KG, Ingelheim am Rhein, Germany
MR 2033 (+)-α-(1R,5R,9R)-5,9-dimethyl-2-(L-tetrahydrofurfuryl)-2′-hydroxy-6,7-benzomorphan
MR 2034 (−)-α-(1R, 5R,9R)-5,9-dimethyl-2-(L-tetrahydrofurfuryl)-2′-hydroxy-6,7-benzomorphan
H. Merz et al., J. Med. Chem., vol. 22, pp. 1475-1479 (1979).

X. Kappa Opioid Agonist available through Preclinical Research, Pharmaceutical Division, Sandoz Ltd., Basel, Switzerland; or Kali-Chemie Pharma Ltd., Hannover, Germany
tifluadom (+)-(1-methyl-2,3-thienyl-carboxyl)-aminomethyl-5-(2-fluorophenyl)-H-2,3-dihydro-1,4-benzodiazepine
D. Romer et al., Life Sciences, vol. 31, pp. 1217-1220 (1982).

XI. Kappa Opioid Agonist available through Centre de Recherches Roussel Uclaf, Romainville, France; or Hop. Sacre-Coeur, Universite de Montreal, Canada
RU 51599 (Niravoline)
G. Hamon et al., J. Am. Soc. Nephrol, vol. 5, p. 272A (1994).

XII. Dynorphin, Dynorphin Derivatives, and Analogs available through Sigma, RBI, ICN, and other pharmaceutical and biochemical distributors:
G. Martinka et al., Eur. J. Pharmacol., vol. 196, pp. 161-167 (1991).
A. Dynorphin A-(1-11)-NH2
B. [D-Ala3]Dyn A(1-11)-NH2
C. [Ala3] Dyn A(1-11)-NH2
F. Lung et al., J. Med. Chem., vol. 38, pp. 585-586 (1995).

XIII. Kappa Opioid Agonist available through EISAI Chemical Co., Tsukuba Research Laboratories, Ibaraki, Japan:
Benzomorphans
ketocyclazocine (+)-3-(cyclopropylmethyl)-8-keto-5-(eq)-9(ax)-dimethyl-6,7-benzomorphan
bremazocine [5R-(5,7,8-β)]-N-methyl-N-[7-(1-pyrrolidinyl)-1-oxaspiro]-4,5-dec-8-yl]-4-benzofuranacetamide

XIV. Other Kappa Opioid Agonists
ethylketocyclazocine (Sterling Winthrop)
HN-11608 (Harslund Nycomed)
RP-60180 (Rorer)
TRK-820 (Toray)
R-84760

As described herein, urinary output can be selectively increased by administration of a kappa opioid receptor agonist to a subject. In exemplary embodiments, the kappa opioid receptor agonist can comprise JT09, nalfurafine, CR665, or difelikefalin.

JT09 (C₃₈H₅₃N₇O₄) can refer to an orally active and peripherally restricted KOA. JT09 increases urine output which reduces serum creatinine levels. JT09 comprises a compound according to Structure (I):

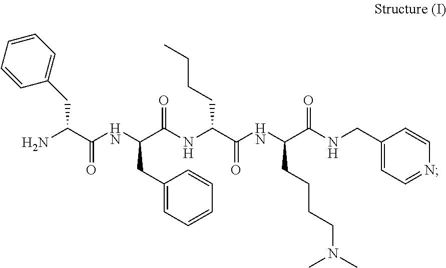 [SMILES: CCCC[C@@H](NC(=O)[C@@H](Cc1ccccc1)NC(=O)[C@H](N)Cc1ccccc1)C(=O)N[C@H](CCCCN(C)C)C(=O)NCc1ccncc1]

Nalfurafine (C₂₈H₃₂N₂O₅) can refer to an orally active, potent, selective, centrally-penetrant κ-opioid receptor (KOR) agonist. Nalfurafine comprises a compound according to Structure (II):

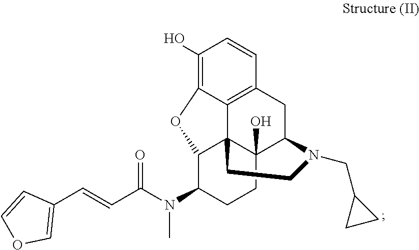

CR665 can refer to a D-amino acid peptide that acts as a peripherally restricted κ-opioid receptor agonist. CR665 comprises a compound according to Structure (III):

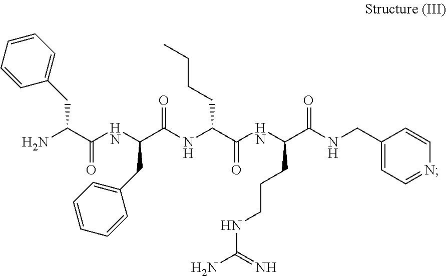 [SMILES: CCCC[C@@H](NC(=O)[C@@H](Cc1ccccc1)NC(=O)[C@H](N)Cc1ccccc1)C(=O)N[C@H](CCCNC(=N)N)C(=O)NCc1ccncc1]

CR845 (also referred to as Difelikefalin) can refer to a peripherally-acting, selective κ-opioid receptor agonist. Difelikefalin comprises a compound according to Structure (IV):

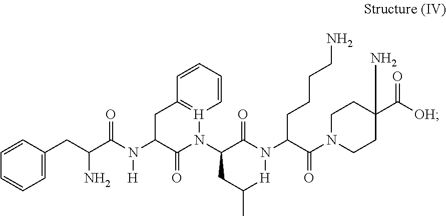

In embodiments, the KOA is an organic molecule. An “organic molecule” can refer to a molecule or compound that contains at least one carbon atom. The organic molecule can be an organic small molecule. A “small molecule” can refer to a chemical compound that is small enough in size so that it can readily pass through a cellular membrane unassisted. In general, a small molecule can refer to a chemical compound that is not a polymer, such as a nucleic acid, polypeptide, or polysaccharide, although the term can encompass small polymers that can readily crossing the cellular membrane.

In embodiments, the kappa opiod agonist can be a peptide or peptidemimetic. The term “peptide” can refer to a macromolecule which comprises a multiplicity of amino or imino acids (or their equivalents) in peptide linkage. In the polypeptide or peptide notation used herein, the left-hand direction is the amino-terminal direction and the right-hand direction is the carboxy-terminal direction, in accordance with standard usage and convention. Peptides can include moieties other than amino acids (e.g., may be glycoproteins, proteoglycans, etc.) and/or may be otherwise processed or modified.

Peptides can contain L-amino acids, D-amino acids, or both and can contain any of a variety of amino acid modifications or analogs known in the art. Useful modifications include, e.g., terminal acetylation, amidation, glycosylation, biotinylation, substitution with D-amino acid or unnatural amino acid, and/or cyclization of the peptide. In some embodiments, peptides may comprise natural amino acids, non-natural amino acids, synthetic amino acids, and combinations thereof. A “short peptide” can refer to any peptide containing up to 25 amino acids (e.g., up to 20, 15, 12, 10, 9, 8, 7, 6, 5, 4, or 3). In some embodiments, a short peptide contains 5-25 amino acids. Peptides also can include peptidomimetics unless indicated otherwise. Herein the terminologies “mimic,” “mimetic,” “peptidomimetic” and the like can be used herein interchangeably. As used herein, an KOA agonistic peptide can refer to any peptide that can directly or indirectly elicit a response in the cell.

Endogenous opioid receptors have been identified in both the central nervous system (brain and spinal cord), and in the periphery. Accordingly, the KOA can be a peripherally acting KOA or a centrally acting KOA.

Non-limiting embodiments of peripherally restricted and/or peripherally acting KOAs include JT09, CR665, and difelikefalin. For example, JT09 is an orally active and peripherally restricted KOA that increases urine output and reduces serum creatinine levels. For example, CR665 is a D-amino acid peptide that acts as a peripherally restricted κ-opioid receptor agonist. For example, difelikefalin is a peripherally-acting, selective κ-opioid receptor agonist

In embodiments, the KOA can be a centrally acting KOA. “Centrally acting” can refer to any compound capable of crossing the blood-brain barrier to exert its effect on the central nervous system (brain and spinal cord). Non-limiting centrally acting KOAs include U-50,488, U-62,066E (spiradoline), CI-977 (enadoline), bremazocine, U-69,593, PD 117302, tifluadom, BRL 52537.

Without wishing to be bound by theory, urinary output can be increased without urinary excretion of electrolytes by administering to a subject a KOA.

In some embodiments, a kappa opioid receptor agonist can be provided as a pharmaceutically acceptable salt. The term “pharmaceutically acceptable” can refer to salts or chelating agents are acceptable from a toxicity viewpoint. The term “pharmaceutically acceptable salt” can refer to ammonium salts, alkali metal salts such as potassium and sodium (including mono, di- and tri-sodium) salts, alkaline earth metal salts such as calcium and magnesium salts, salts with organic bases such as dicyclohexylamine salts, N-methyl-D-glucamine, and salts with amino acids such as arginine, lysine, and so forth.

The term “salt”, as used herein, denotes acidic and/or basic salts, formed with inorganic or organic acids and/or bases, for example basic salts. While pharmaceutically acceptable salts can be used when employing the compounds of the invention as medicaments, other salts can be used as well, for example, in processing these compounds, or for non-medicament-type uses. Salts of these compounds can be prepared by art-recognized techniques. Examples of such pharmaceutically acceptable salts include, but are not limited to, inorganic and organic acid addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well. “Pharmaceutically acceptable anions” as used herein includes the group consisting of CH₃COO⁻, CF₃COO⁻, Cl⁻, SO₃²⁻, maleate and oleate.

Aspects of the inventions are also drawn towards combination compositions and methods comprise at least one KOA and at least one additional active agent. In embodiments, the at least one additional active agent can be a diuretic or an anti-hypertensive agent. The skilled artisan will recognize that the dosage of the at least one additional active agent can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the biological effect desired; and rate of excretion.

The term “diuretic” can refer to any compound or substance that assist in diuresis (i.e., the process of increasing urine production or excretion). For example, the diuretic can be a loop diuretic, a thiazide or thiazide-like diuretic, or a potassium sparing diuretic. As described herein, combining a kappa opioid receptor agonist with a diuretics (non-limiting examples of standard-of-care diuretics include furosemide, torsemide, ethacrynic acid, bumetanide, hydrochlorothiazide, chlorthalidone, metolazone, indapamide, chlorothiazide, or bendroflurnethiazide, amiloride, triamterene, spironolactone, or eplerenone) increases the magnitude and duration of the diuresis and decreases the amount of sodium and potassium excreted into the urine when compared to the standard-of-care diuretic alone.

A “loop diuretic” can refer to diuretics that can affect the ascending limb of the loop of Henle in the kidney. Non-limiting examples of loop diuretics comprise furosemide, torsemide, ethacrynic acid, or bumetanide.

A “thiazide diuretic” or “thiazide-like diuretic” can refer to a compound that comprises a benzothiadiazine molecular structure. In some embodiments, thiazide diuretics inhibit sodium and chloride reabsorption in the distal tubules of the kidney, resulting in increased sodium and water excretion in the urine. Non-limiting examples of thiazide or thiazide-like diuretics comprise hydrochlorothiazide, chlorthalidone, metolazone, indapamide, chlorothiazide, bendroflumethiazide, altizide, cyclopentiazide, cyclothiazide, epitizide, hydroflumethiazide, mebutizide, meticlothiazide, polythiazide, and trichlormethiazide.

A “potassium sparing diuretic” can refer to compounds that increase diuresis (urination) without the loss of potassium. They are generally weak diuretics and work by interfering with the sodium-potassium exchange in the distal convoluted tubule of the kidneys or as an antagonist at the aldosterone receptor. Non-limiting examples of a potassium sparing diuretic comprise amiloride, triamterene, spironolactone, and eplerenone.

Classical diuretics (thiazide and loop diuretics) produce an increase in urine output by acting within the kidneys to prevent the renal tubular reabsorption of sodium, which over time can lead to diuretic-induced hyponatremia, hypokalemia, and activation of compensatory mechanisms (e.g., increased renal tubular sodium reabsorption in nephron segments not blocked by the diuretic, central release of antidiuretic hormone) which lead to loss of diuretic efficacy (diuretic resistance).

The term “anti-hypertensive agent” can refer to any compound that when administered to a subject reduces blood pressure. Non-limiting examples of anti-hypertensives comprise an angiotensin receptor blocking agent, an angiotensin converting enzyme inhibitor, a beta blocker, and alpha receptor blocker, a centrally acting sympatholytic, a calcium channel blocker, or a direct acting vascular smooth muscle dilator.

In some embodiments, the compounds can be present in a pharmaceutical composition comprising a pharmaceutically acceptable excipient, carrier, or diluent. In embodiments, a pharmaceutically acceptable excipient, carrier, or diluent can comprise any solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Accordingly, embodiments of the invention comprise combination compositions comprising kappa opioid receptor agonists and at least one additional active agent. A “combination composition” can refer to a composition comprising a mixture of at least two different active compounds.

Aspects of the invention are drawn to the use of compositions described herein for the treatment of a subject afflicted with a cardiovascular disease or a renal disease.

In embodiments, the term “subject” or “patient” can refer to any organism to which aspects of the invention can be administered, e.g., for experimental, diagnostic, prophylactic, and/or therapeutic purposes. Typical subjects to which compounds described herein can be administered will be mammals, for example primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals, for example, pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted herein or another organism that is alive. The term “living subject” refers to the entire subject or organism and not just a part excised (e.g., a liver or other organ) from the living subject. The terms “subject”, “individual”, and “patient” can be used interchangeably.

The term “treating” or “to treat” can refer to clinical intervention in an attempt to alter the natural course of the individual or subject being treated. For example, “treating a disease” can comprise partially or completely alleviating, ameliorating, improving, relieving, delaying onset of, inhibiting progression of, reducing severity of, and/or reducing incidence of one or more symptoms, features, or clinical manifestations of a disease, disorder, and/or condition, such as cardiovascular disease or renal disease. Clinical indications of treating or preventing a disease can comprise, for example, increasing urine output, decreasing urinary sodium, potassium and chloride excretion, decreasing urine osmolality, decreasing mean arterial pressure, or a combination thereof.

Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition.

A “cardiovascular disease” can refer to a disease or condition that affects the heart or blood vessels. In embodiments, a cardiovascular disease can involve the kidneys. In embodiments, cardiovascular disease can include a disease caused or exacerbated by cardiovascular and renal complications (e.g., example hypertension, heart failure, cardiorenal syndrome).

The term “heart failure” can refer to the pathophysiologic state in which an abnormality of cardiac function is responsible for the failure of the heart to pump blood at a rate commensurate with the requirements of the metabolizing tissues.

The term “hypertension” can refer to elevation of systemic and/or diastolic blood pressure. Hypertension can include essential, or primary, hypertension wherein the cause is not known or where hypertension is due to greater than one cause, such as changes in both the heart and blood vessels; and secondary hypertension wherein the cause is known. Causes of secondary hypertension include but are not limited to obesity; kidney disease; hormonal disorders; use of certain drugs, such as oral contraceptives, corticosteroids, cyclosporin, and the like. Clinically, hypertension can be indicated by high blood pressure, in which both the systolic and diastolic pressure levels are elevated (>130 mmHg/>80 mmHg), and isolated systolic hypertension, in which only the systolic pressure is elevated to greater than or equal to 130 mm Hg, while the diastolic pressure is less than 80 mm Hg. Normal blood pressure can be defined as less than 120 mm Hg (systolic pressure) and less than 80 mm Hg (diastolic pressure). A hypertensive subject is a subject with hypertension. A pre-hypertensive subject is a subject with an elevated blood pressure that is between 120 mmHg less than 80 mmHg and 129 mmHg and less than 80 mmHg. One outcome of treatment is decreasing mean arterial pressure in a subject.

A “renal disease” can refer to a disease or condition related to reduction in kidney function compared to healthy kidney function. A renal disease can include a disease caused or exacerbated by renal complications. For example, the renal disease can be chronic kidney disease. The term “chronic kidney disease” or “chronic renal disease” can refer to a disease or condition related to the progressive reduction in kidney function compared to healthy kidney function. Chronic kidney disease can be characterized by a glomerular filtration rate (GFR) of less than 90 ml/min/1.73 m2 for three or more months or kidney damage (e.g., presence of high levels of protein in the urine, such as albumin). In embodiments, chronic kidney disease is stage 1 wherein glomerular filtration rate (GFR) is 90-120 ml/min/1.73 m2 but there is radiologic or other evidence of kidney disease (such as protein in the urine). In embodiments, chronic kidney disease is stage 2 wherein glomerular filtration rate (GFR) is from 60 to 89 ml/min/1.73 m2. In embodiments, chronic kidney disease is stage 3A wherein glomerular filtration rate (GFR) is from 45 to 59 ml/min/1.73 m2. In embodiments, chronic kidney disease is stage 3B wherein glomerular filtration rate (GFR) is from 30 to 44 ml/min/1.73 m2. In embodiments, chronic kidney disease is stage 4 wherein glomerular filtration rate (GFR) is from 15 to 29 ml/min/1.73 m2. In embodiments, chronic kidney disease is stage 5 wherein glomerular filtration rate (GFR) is less than 15 ml/min/1.73 m2, which is also called “end stage renal disease” or ESRD. A normal (e.g. healthy) glomerular filtration rate may be greater than or equal to 90 ml/min/1.73 m2. A normal (e.g. healthy) glomerular filtration rate may be 90 to 120 ml/min/1.73 m2. In embodiments, an average normal GFR (e.g., not associated with chronic kidney disease) associated with age (age in years: GFR) is 20-29:116, 30-39:107, 40-49:99, 50-59:93, 60-69:85, greater than 70:75. In one embodiment, wherein renal disease is not polycystic kidney disease.

Aspects of the invention are drawn to compositions and methods to selective increase urine output (i.e., promote urinary output) while reducing or preventing change in urinary excretion of electrolytes.

Aspects of the invention are drawn to compositions and methods to sensitize a subject to a diuretic, wherein the subject suffers from diuretic resistance and/or the subject has failed to achieve diuresis with diuretic therapy prior to administering the KOA.

The term “sensitize” can refer to raising the sensitivity or reducing the resistance of a subject to a therapeutic treatment, such as a diuretic.

The term “diuretic resistance” can refer to a condition in which the subject does not respond, or has a poor response, to treatment with a diuretic. For example, the subject does not respond, or has a poor response, to increase fluid and sodium output by the kidneys sufficiently to relieve volume overload, edema, or congestion, despite treatment with a diuretic, such as a loop diuretic. One method to diagnose diuretic resistance is the failure to excrete at least 90 mmol of sodium within 72 hrs of an oral furosemide dose (for example, 80 mg) given twice a day. Normal response to a diuretic has been defined as producing 3-4 L of urine per 40 mg of furosemide, whereas diuretic resistant individuals exhibit impaired capacity below this level. This effect can be caused by one or combination of mechanisms: (i) a change in the pharmacokinetic profile of loop diuretics, (ii) compensation of sodium reabsorption at proximal and distal nephron sites not targeted by the diuretic, and (iii) diminished nephron response.

Embodiments as described herein can comprise administering to the subject a composition comprising a kappa opioid receptor agonist. For example, the kappa opioid agonists can reverse and/or prevent diuretic resistance. Exemplary compounds are described herein.

The term “administration”, “administer”, or “administering” can refer to the physical introduction to the subject of a composition containing a therapeutic agent using any of a variety of methods and delivery systems known to those of skill in the art.

Embodiments can comprise the administration of a kappa opioid receptor agonist and at least one additional active agent, such as a diuretic or an anti-hypertensive agent. In embodiments, the at least one additional active agent can be administered sequentially or concurrently.

“Sequential administration” can refer temporally separated administration of the agents or therapies described herein. For example, the first and second therapies can be administered during a combination therapy for a time interval greater than about 15 minutes (about 20 minutes, 30 minutes, 40 minutes, 50 minutes, 60 minutes, or longer).

“Concurrent administration” can refer to the simultaneous administration of two agents in any manner in which the pharmacological effects of the two agents are apparent in the patient. For simultaneous administration, the two agents need not be administered as a single pharmaceutical composition, in the same dosage form, or by the same route of administration.

The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

In embodiments, the composition can be administered peripherally. “Peripheral administration” can refer to any route of administration that is applied outside the blood-brain barrier. In embodiments, peripherally administered agonists can cross the blood brain barrier.

Embodiments as described herein can be administered to a subject in the form of a pharmaceutical compositions suitable for the intended route of administration. Such compositions can comprise, for example, the active ingredient, such as the kappa opioid receptor agonist, and a pharmaceutically acceptable carrier, excipient, or diluent. Such compositions can be in a form adapted to oral, subcutaneous, parenteral (intravenous, intraperitoneal, intradermal), intramuscular, rectal, epidural, intratracheal, inhalation, intranasal, transdermal (i.e., topical), transmucosal, vaginal, buccal, ocularly, or pulmonary administration, for example, in a form adapted for administration by a peripheral route, or is suitable for oral administration or suitable for parenteral administration. Other routes of administration are subcutaneous, intraperitoneal and intravenous, and such compositions can be prepared in a manner well-known to the person skilled in the art, e.g., as described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions can appear in conventional forms, for example, solutions and suspensions for injection, capsules and tablets, such as in the form of enteric formulations, e.g. as disclosed in U.S. Pat. No. 5,350,741, for oral administration.

In embodiments, the pharmaceutical carrier, excipient, or diluent can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. The carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier will vary widely but can be, for example, from about 1 mg to about 1 g.

When a liquid carrier is used, the preparation can be in the form of a syrup, emulsion, soft gelatin capsule or sterile injectable liquid such as an aqueous or non-aqueous liquid suspension or solution.

The composition can also be in a form suited for local or systemic injection or infusion and can, as such, be formulated with sterile water or an isotonic saline or glucose solution. The compositions can be in a form adapted for peripheral administration only, with the exception of centrally administrable forms. The compositions can be in a form adapted for central administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™M (BASF, Parsippany, N.J.) or phosphate buffered saline (PBS). The composition can be sterile and can be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, the kappa opioid receptor agonist can be administered by bolus injection or by infusion. A bolus injection can refer to a route of administration in which a syrine is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration of the kappa opioid receptor agonist or nociceptin opioid peptide receptor agonist in a continuous manner, such as to maintain the therapeutic effect (activity) over a prolonged period of time.

The compositions can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The composition can contain pharmaceutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

Multi-dose formulations can be prepared as a solution of a compound of the invention in sterile, isotonic saline, stored in capped vials, and if necessary a preservative is added (e.g. benzoates). Fixed dose formulations can be prepared as a solution of the compound in sterile, isotonic saline, stored in glass ampoules, and if necessary filled with an inert gas. Each dose of the compound is stored dry in ampoules or capped vials, if necessary filled with inert gas. The multi-dose formulation demands the highest degree of stability of the compound. When the stability of the compound is low fixed dose formulations can be used. For nasal administration, the preparation can contain a compound of the invention dissolved or suspended in a liquid carrier, for example, an aqueous carrier, for aerosol application. The carrier can contain additives such as solubilizing agents, e.g., propylene glycol, surfactants such as bile acid salts or polyoxyethylene higher alcohol ethers, absorption enhancers such as lecithin (phosphatidylcholine) or cyclodextrin, or preservatives such as parabines.

A “therapeutically effective amount” or “therapeutically effective dose” can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

In embodiments, a therapeutically effective amount, such as a therapeutically effective amount of a kappa opioid receptor agonist, can comprise a dose of about 0.005 mg/kg to about 1000 mg/kg. In some embodiments, a therapeutically effective amount can comprise a dose of about 0.005 mg/kg to about 10 mg/kg. In some embodiments, a therapeutically effective amount can comprise a dose of about 0.25 mg/kg to about 2 mg/kg. In some embodiments, the therapeutically effective amount is at least about 0.001 mg/kg at least about 0.0025 mg/kg, at least about 0.005 mg/kg, at least about 0.01 mg/kg, at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. However, the skilled artisan will recognize that the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the biological effect desired; and rate of excretion.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Kappa opioid receptor agonists administered to animals produce an increase in urine output and a decrease in urinary sodium and potassium excretion (water diuresis), while also generating a decrease in blood pressure through the production of nitric oxide in vascular endothelium. However, the translation of these therapies into the clinic has been hindered by psychotomimetic adverse effects. Nalfurafine is a G-protein biased kappa opioid receptor agonist that has been approved for treatment-resistant uremic pruritis in Japan and is currently in a number of clinical trials in the US. Because nalfurafine is G-protein biased, it is able to provide most of the beneficial effects of classical kappa opioid agonists, while circumventing the psychotomimesis produced by classical kappa opioid receptor agonists. Recently, the diuretic effect of nalfurafine has been described in the literature, however the cardiovascular response remains unknown. Our laboratory has demonstrated that nalfurafine causes a significant decrease in blood pressure when administered to normotensive rats, while also producing a water diuresis. Our lab has also shown that combining nalfurafine with standard-of-care diuretics (furosemide, hydrochlorothiazide) increases the magnitude and duration of the diuresis and decreases the amount of sodium and potassium excreted into the urine when compared to the standard-of-care diuretic alone. Hypertension is a complicated disease that usually requires a combination of therapies that act on different parameters that contribute to the disease process. These combinations typically involve a diuretic, a vasodilator, an angiotensin converting enzyme (ACE) inhibitor/angiotensin receptor blocker (ARB), or a β-adrenoreceptor antagonist. Therefore, without wishing to be bound by theory, nalfurafine alone or in combination with other antihypertensive medications could provide an effective treatment for hypertension. Aspects of the invention are drawn to: 1) Nalfurafine, administered acutely or chronically, decreases blood pressure in normotensive and hypertensive settings and can be used to effectively treat hypertension, and 2) When administered in combination, kappa opioid agonists increase the diuretic response produced by standard-of-care diuretics, while limiting adverse hyponatremic/hypokalemic effects typically caused by these classical diuretics.

Nalfurafine can be used for the acute and chronic management of hypertension. Kappa opioid receptor agonists can be combined with standard-of-care diuretics to increase diuretic efficacy, prevent diuretic resistance or tolerance, reverse diuretic resistance or tolerance, and decrease adverse effects caused by classical diuretics.

Classical diuretics (thiazide and loop diuretics) produce an increase in urine output by acting within the kidneys to prevent the renal tubular reabsorption of sodium, which over time can lead to diuretic-induced hyponatremia, hypokalemia, and activation of compensatory mechanisms (e.g., central release of antidiuretic hormone) which lead to loss of diuretic efficacy (diuretic tolerance). In contrast, nalfurafine is thought to cause diuresis by acting in the brain to inhibit the release/secretion of antidiuretic hormone, while having no effect on the urinary excretion of sodium or potassium. Thus, administration of nalfurafine together with a loop/thiazide diuretic would afford the unique effect of selectively enhancing the renal excretion of water, but not sodium/potassium, to a level greater than the diuresis produced by either drug administered alone. Further, the ability of nalfurafine to retain sodium and potassium will counter the loss of these electrolytes caused by loop and thiazide diuretics. Thus together, the combination of nalfurafine and a loop or thiazide diuretic will have a major therapeutic advantage in producing a marked and selective water diuresis without adverse hyponatremic/hypokalemic adverse effects that can be sustained chronically with minimal risk for development of diuretic resistance. Finally, in addition to producing water diuresis, the action of nalfurafine to also decrease blood pressure will be a major advantage toward treatment of high blood pressure, since a single medication can target two contributing factors (water retention and vascular smooth muscle contraction) that are recognized to contribute to the etiology of hypertension. Nalfurafine is also different than other kappa opioid receptor agonists in that it does not produce psychotomimetic effects. Thus, combining kappa opioid receptor agonists with standard-of-care diuretics improves upon a widely successful class of therapeutics that is prescribed daily and could reduce the amount of adverse events associated with these medications.

### Example 2—Nalfurafine Combined With Standard-of-Care Diuretics Increases Diuresis and Limits Electrolyte Losses

Kappa opioid receptor (KOR) agonists produce a variety of beneficial effects, including a water diuresis, but their translation into the clinic has been hindered by psychotomimetic adverse effects. Nalfurafine is a novel, G protein biased KOR agonist that has been shown to produce several desired effects of KOR agonists, while avoiding central adverse effects. To validate the clinical use of this drug, this study examined the cardiovascular and renal responses to i.v. nalfurafine alone or in combination with the clinically used diuretics: furosemide, hydrochlorothiazide (HCTZ), and amiloride. Following chronic instrumentation, conscious Sprague-Dawley rats were continuously infused i.v. with isotonic saline; after stabilization, rats were administered i.v. bolus nalfurafine, diuretics, diuretics combined with nalfurafine, or vehicle, and mean arterial pressure (MAP), heart rate (HR), and urine output were recorded for 90 min. IV nalfurafine produced a marked diuresis, antinatriuresis, antikaliuresis, and decrease in MAP without eliciting a change in HR. As compared to diuretic treatment alone, co-administration of nalfurafine notably increased the total urine output to furosemide and HCTZ while reducing the amount of sodium and potassium excreted. When combined with amiloride, nalfurafine also increased the diuresis and decreased the amount of sodium excreted. In contrast to these diuretics administered alone, MAP was reduced with nalfurafine combination therapy. Together, these findings demonstrate that nalfurafine has a clinically important action to augment the diuresis to classical diuretics without causing excessive loss of electrolytes characteristic of these drugs. Without wishing to be bound by theory, combination therapy of nalfurafine with loop/thiazide diuretics can offer a new approach to treat several cardiovascular conditions such as hypertension, volume overloaded states, and electrolyte abnormalities.

### Example 3—Nalfurafine Combined With Standard-of-Care Diuretics Increases Diuresis and Limits Electrolyte Losses

Kappa opioid receptor (KOR) agonists produce a water diuresis, but their clinical utility has been hampered due to psychomimetic adverse effects. Standard-of-care loop and thiazide diuretics offer mainstay therapy for treatment of volume overload states, but these drugs often produce serious electrolyte adverse effects.

As described herein, intravenous bolus injection and oral (gavage) administration of nalfurafine, a KOR agonist without adverse central effects, produced a marked increase in urine output and decrease in urinary sodium/potassium excretion. Co-administration of nalfurafine with furosemide, hydrochlorothiazide, or amiloride augmented the total urine output and markedly reduced electrolyte losses caused by these diuretics. Thus, Nalfurafine administration alone and in combination with standard-of-care diuretics is a new therapy for treatment of cardiovascular and renal disease.

Abbreviations used in this example: KOR, kappa opioid receptor; i.v., intravenous; MAP, mean arterial pressure; HR, heart rate; HCTZ, hydrochlorothiazide; V, urine flow rate; U_{Na}V, urinary sodium excretion rate; U_{K}V, urinary potassium excretion rate.

Abstract: Kappa opioid receptor (KOR) agonists have therapeutic potential as antihypertensive and water diuretic (aquaretic) agents, yet adverse psychotomimetic effects have hindered their clinical development. Nalfurafine is a KOR agonist reported to be devoid of central adverse effects. Therefore, this study validated the cardiovascular and renal responses to nalfurafine. Also, nalfurafine was combined with standard-of-care diuretics to explore which channels nalfurafine utilizes to alter the renal handling of electrolytes and to determine this drug's clinical potential.

Experimental Approach: Changes in cardiovascular and renal excretory function were measured in conscious rats before and after (90-min) i.v. bolus injection of nalfurafine, furosemide, hydrochlorothiazide, amiloride, or nalfurafine combined with a diuretic. In separate studies, changes in urine output and the renal excretion of sodium/potassium produced by oral (gavage) administration of nalfurafine alone and in combination with diuretics were examined (5-hrs).

Results: Intravenous and oral nalfurafine produced a marked diuresis, antinatriuresis, and antikaliuresis. Nalfurafine also augmented the total urine output produced by i.v. and oral furosemide, hydrochlorothiazide, and amiloride and markedly decreased the sodium and potassium excreted by each diuretic. Nalfurafine decreased blood pressure alone and in combination with these diuretics.

Conclusions: Nalfurafine produced a significant aquaresis and slight reduction in blood pressure in rats. Nalfurafine also increased total urine output produced by standard-of-care diuretics while attenuating electrolyte losses and lowering blood pressure. Without wishing to be bound by theory, combination therapy of nalfurafine with a loop/thiazide diuretic offers a new approach to treat volume overload states while minimizing adverse electrolyte derangements caused by standard-of-care diuretics.

Introduction: Diuretics are one of the most prescribed therapies throughout the world and have proven to be an effective first-line class of antihypertensive drugs. However, the thiazide and loop classes of diuretics are well known to cause many adverse effects, including severe and potentially dangerous electrolyte abnormalities. Thiazide diuretics are common causes of hyponatremia (Krogager, Mortensen, et al., 2020) and combining thiazide diuretics, such as hydrochlorothiazide, with other antihypertensive medications increases the likelihood of developing hypokalemia (Van Blijderveen, Straus, et al., 2014). Loop diuretics are one of the cornerstone treatments for lowering blood pressure and removing edema. However, use of loop diuretics in heart failure patients, particularly at high doses, is likely to produce hyponatremia and/or hypokalemia (Kapelios, 2015; Velat, Bušić, et al, 2020). Further, resistance to the action of thiazide and loop diuretics, which leads to residual edema and congestion is frequently encountered in the clinical setting and is a known predictor of worsening outcomes (Kiernan, Stevens, et al., 2018).

Given the widespread use and serious complications of these drugs, stabilizing electrolyte losses caused by diuretics is of high clinical significance. However, an effective therapeutic strategy would not only limit electrolyte disturbances, but also help to improve the overall pattern of the diuresis while avoiding onset of diuretic resistance.

Kappa opioid receptor (KOR) agonists are well known to produce a water diuresis (aquaresis), wherein the increase in urine output is associated with a concurrent reduction in urinary sodium and potassium excretion and decrease in urine osmolality (Inan, Lee, et al., 2009). KOR agonists also produce a variety of other beneficial effects such as nonaddictive antinociception and organ protection from ischemia/reperfusion injury, but their translation into the clinic has been stalled by the psychotomimetic adverse effects of this class of drugs (Beck, Reichel, et al., 2019; Liu, Yu, et al., 2018; Pfeiffer, Brantl, et al., 1986). One exception, however, is nalfurafine, which is a selective KOR agonist that has been approved in Japan for clinical use and is currently prescribed for treatment-resistant pruritus in patients with renal failure undergoing hemodialysis (Kumagai, Ebata et al., 2010). Nalfurafine is a G-protein biased KOR agonist, which preferentially activates downstream Galpha-subunit protein in lieu of β-arrestin signaling pathways (Kaski, White, et al., 2019; Schattauer, Kuhar, et al., 2017). The psychotomimetic effects of KORs have been linked to β-arrestin signaling, suggesting that nalfurafine could produce the beneficial G-protein-linked effects of KORs while avoiding the undesirable aspects of β-arrestin signaling (Ehrich, Messinger, et al., 2015). Additionally, a post-marketing surveillance study of nearly 4000 hemodialysis patients demonstrated that nalfurafine did not cause the typical dysphoric adverse effects of standard KOR agonists (Kozono, Yoshitani, et al., 2018). While the nociceptive and signaling properties of nalfurafine are well characterized, the cardiovascular and renal effects remain largely unexplored.

The present studies were performed to validate the cardiovascular and renal responses produced by the i.v. bolus and oral (gavage) administration of nalfurafine in conscious Sprague-Dawley rats. Additionally, experiments were performed in which nalfurafine was administered in combination with standard-of-care diuretics to validate that nalfurafine alters the renal handling of sodium and potassium. The diuretics furosemide, hydrochlorothiazide, and amiloride were chosen given their known ability to inhibit electrolyte transport at different nephron segments and to validate the efficacy of nalfurafine to minimize electrolyte losses caused by these clinically used diuretics.

### Methods:

Animals: Male Sprague-Dawley (SD) rats (Envigo Labs, Indianapolis, IN), 300-350 g, were individually housed under a 12 h light-dark cycle and allowed tap water and standard rodent diet ad libitum. Rats were randomly assigned to experimental groups for all acute and subacute studies. All procedures were approved by the Louisiana State University Health Sciences Center Institutional Animal Care and Use Committee and conducted in accordance with the National Institutes of Health guidelines for the care and use of animals.

Surgical Procedures: Aseptic technique was followed for all surgical procedures. On the morning of the experiment, rats were anesthetized with and maintained under inhaled 2% isoflurane, and polyethylene catheters (PE50; BD Diagnostics, Sparks, MD) were surgically inserted into the left femoral vein and left femoral artery for the infusion of saline and/or drug administration and the recording of arterial blood pressure, respectively. Subsequently, a suprapubic incision was made and a flanged polyethylene catheter (PE240; BD Diagnostics, Sparks, MD) was inserted into the bladder, exteriorized, and sutured to surrounding muscle and skin. Rats were then placed in an elevated restrainer (Krowicki & Kapusta, 2011) to recover from surgery and allow collection of urine. When acute experimental protocols were completed, rats were anesthetized via isoflurane and decapitated.

Acute Cardiovascular and Renal Excretory Studies: Following surgery, rats received an i.v. infusion of isotonic saline at 55 μL min⁻¹ throughout the duration of the experiment and were allowed 4-6 hours to recover from anesthesia. Arterial catheters were connected to a pressure transducer (MLT0670, ADInstruments, Sydney, Australia), which was connected to Powerlab 8/30 (ADInstruments) for recording of pulsatile and mean arterial blood pressure (MAP) and heart rate (HR). Blood pressure and HR were visualized using LabChart software (v8.1.5; ADInstruments). Next, urine was collected every 10 minutes for a control period of 30 minutes until three consecutive urine rates of 55±15 μL min⁻¹ were observed. Following this control period, drug or vehicle (isotonic saline) was injected into the femoral vein catheter at a volume of 2 cc kg⁻¹, and urines were collected in 10 minute periods for a span of 90 minutes. The following drug concentrations were used for these experiments: nalfurafine 5 μg kg⁻¹, furosemide 7.5 mg kg⁻¹, hydrochlorothiazide 2.5 mg kg⁻¹, and amiloride 2 mg kg⁻¹. All groups are n=6. Urine volume was measured gravimetrically, urine sodium and potassium concentrations were measured using flame photometry (model 943; Instrumentation Laboratories, Lexington, MA), and urine osmolality was measured using a vapor pressure osmometer (model 5600; WESCOR, Logan, UT).

Metabolic Cage Renal Excretory Studies: Rats were placed in metabolic cages (model 18cv, Fenco, Cataumet, MA) for 2 hours to allow for acclimation and voiding of residual urine. Following the acclimation period, rats were volume loaded with 20 cc kg⁻¹ drug or vehicle (isotonic saline) via oral gavage. Urines were collected each hour over a 5-hour period. Following completion of the study rats were returned to their home cages. The following drug concentrations were used for these experiments: nalfurafine 150 μg kg⁻¹, furosemide 50 mg kg⁻¹, hydrochlorothiazide 5 mg kg⁻¹, and amiloride 5 mg kg⁻¹. All groups are n=6, except for urinary sodium and potassium excretion rate time points where rats did not urinate during that time point. These points are marked on the figures with number of measurements in parentheses.

Experimental Design and Analysis: All results are expressed as mean±SEM. In the acute i.v. bolus cardiovascular and renal excretory studies, changes at time points within a treatment group were compared to control value (C) using two-way analysis of variance (ANOVA) with repeated measures, and a post hoc Dunnett's test was used following confirmation of a significant F value. For changes between treatment groups, values at respective time points were compared using two-way ANOVA with repeated measures, and a post hoc Sidak's test was used following confirmation of a significant F value. In the subacute renal excretory studies, changes between treatment groups were compared using mixed linear modeling with repeated measures, and a post hoc Sidak's test was used following confirmation of a significant F value. Means between groups for total urine output, Na⁺ concentration, K⁺ concentration, and urine osmolality were compared using a Student's t-test. Statistical significance was characterized as P<0.05. Statistics were carried out using Prism 7.05 software (GraphPad Software, Inc., San Diego, CA) and IBM SPSS Statistics (IBM, Armonk, NY).

Materials: Nalfurafine was provided by Med Chem Express, Monmouth Junction, NJ; hydrochlorothiazide and amiloride by Sigma Aldrich, St. Louis, MO; furosemide by Tokyo Chemical Industry, Tokyo, Japan.

### Results

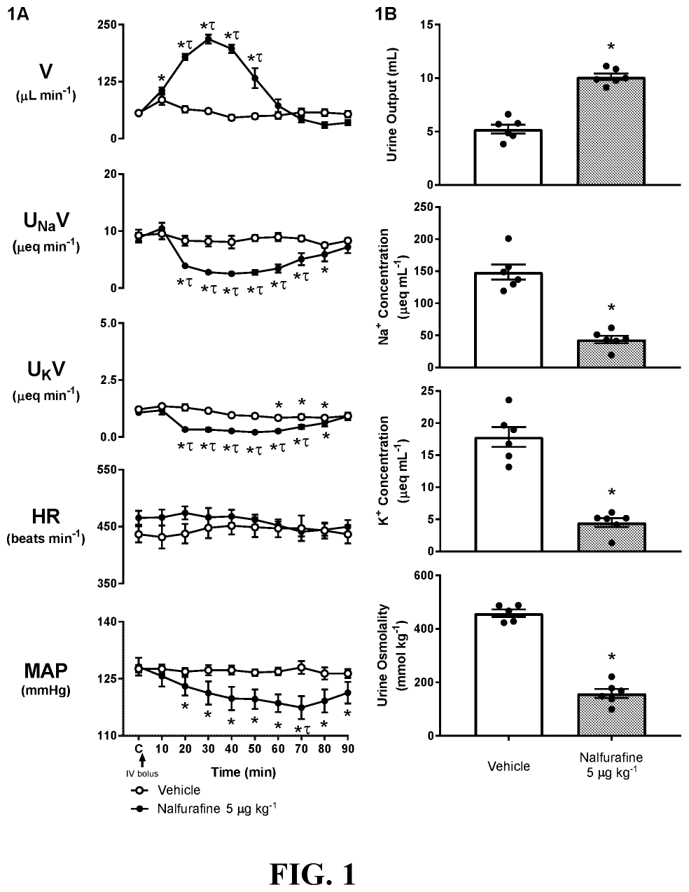

Cardiovascular and Renal Excretory Responses to Nalfurafine: The cardiovascular and renal excretory responses produced by i.v. bolus injection of nalfurafine (5 μg kg⁻¹) in conscious Sprague-Dawley rats are shown in FIG. 1A. Nalfurafine elicited a significant increase in urine flow rate compared to the respective group control value (C) which started 10-min following i.v. injection and persisted for 50 min, with peak diuresis occurring at 30 min. Urine flow rate was significantly different when compared to saline vehicle treated animals starting at 20 min and ending at 50 min. The cumulative amount of urine excreted throughout the 90-min protocol was significantly higher in the nalfurafine treated group compared to vehicle treated animals (FIG. 1B; 10.12±0 0.30 mL vs 5.24±0.40 mL). Concurrent with the increase in urine flow rate, nalfurafine produced a significant antinatriuresis and antikaliuresis compared to respective control values beginning at 20 min and ending at 80 min; peak antinatriuresis occurred at 40 min and peak antikaliuresis occurred at 50 min (FIG. 1A). Urinary sodium and potassium excretion rates were significantly lower in the nalfurafine group when compared to vehicle beginning at 20 min and ending at 70 min. Further, the concentration of sodium and potassium and osmolality measured in the cumulative 90-min urine sample was significantly less in the nalfurafine as compared to vehicle treated animals (FIG. 1B: [Na+] 43.59±5.71 μeq ml⁻¹ vs 148.8±11.76 μeq ml⁻¹; [K+] 4.51±0.68 μeq ml⁻¹ vs 17.83±1.52 μeq ml⁻¹; Urine Osmolality; 158.7±16.78 mmol kg⁻¹vs 458.6±14.04 mmol kg⁻¹, respectively). Notably, when compared to control levels, nalfurafine produced a slight, but significant decrease in MAP beginning 20-min after drug administration and lasting throughout the protocol (FIG. 1A; 70-min peak Δ; −11 mmHg). When compared to saline, nalfurafine produced a significantly different MAP at the 70-min time point. Interestingly, despite the decrease in MAP, nalfurafine did not change HR throughout the study.

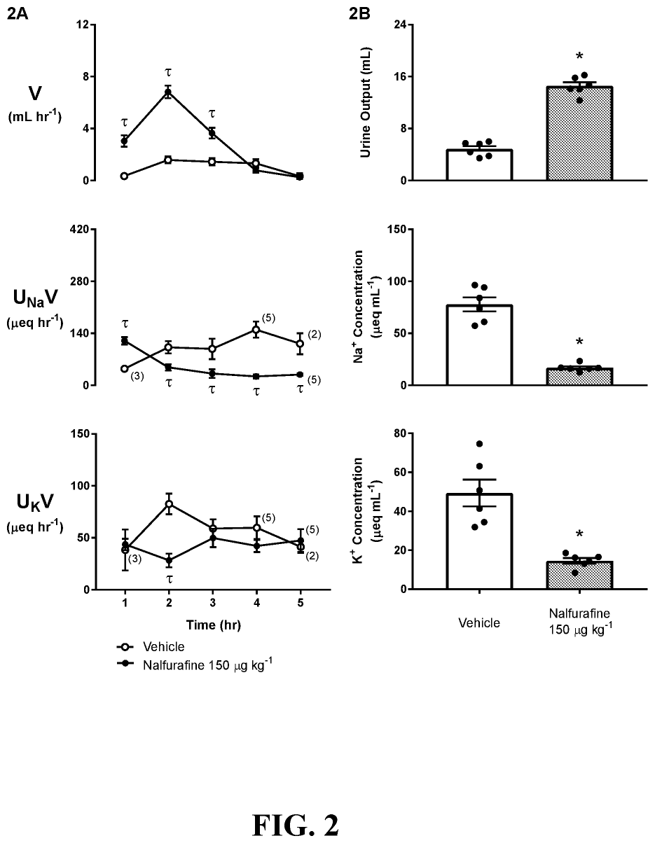

The renal excretory effects produced by oral administration (gavage) of nalfurafine (150 μg kg⁻¹) or vehicle (saline; 20 mL kg⁻¹) in conscious rats are displayed in FIG. 2A and FIG. 2B. Oral administration of nalfurafine produced a significant increase in urine flow rate compared to saline starting at hour-1 and ending at hour-3 post gavage, with peak diuresis occurring at hour-2 (FIG. 2A). The nalfurafine treated group excreted significantly more total urine compared to the saline treated group (FIG. 2B: 14.55±0.57mL vs 4.8±0.45 mL, respectively). In terms of the renal handling of sodium, in the first hour nalfurafine treated animals excreted sodium at a significantly higher rate than saline treated animals, but thereafter (hours 2-5) excreted sodium at a notably lower rate than saline treated animals. The concentration of sodium measured in the cumulative 5-hour urine collection was significantly lower than nalfurafine than saline treated animals (FIG. 2B: 16.92±1.45 μeq ml⁻¹ vs 77.81±6.78 μeq ml⁻¹, respectively). Interestingly, nalfurafine only produced a significant difference in urinary potassium excretion at the 2 hour time point when compared to saline; however, the total potassium concentration excreted between nalfurafine and saline was significantly different (14.55±1.46 μeq ml⁻¹ vs 49.39±6.89 μeq ml⁻¹).

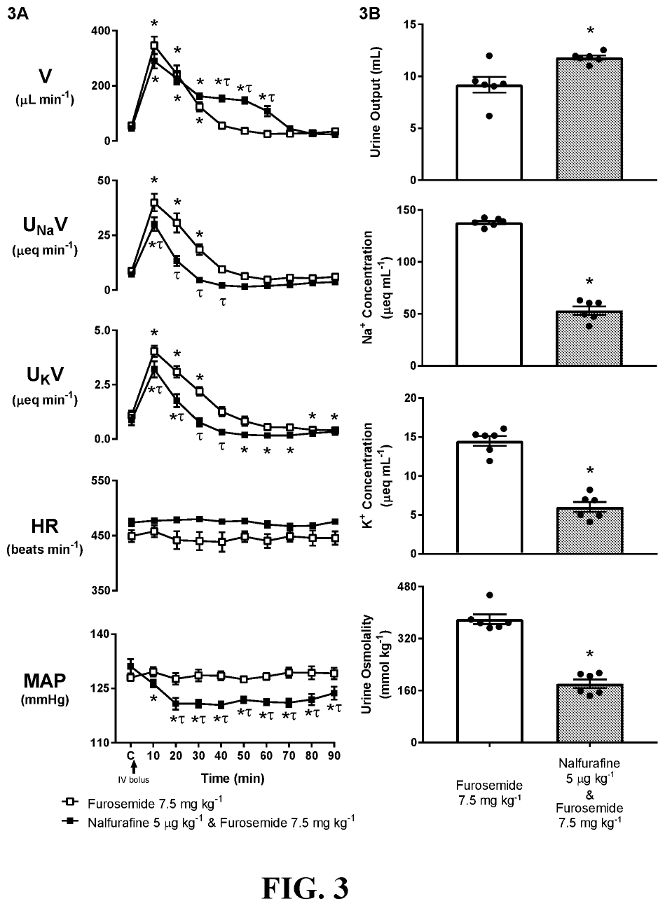

Cardiovascular and Renal Excretory Responses to Co-Administration of Furosemide and Nalfurafine: FIG. 3A displays the time-course changes in cardiovascular and renal excretory function produced by i.v. furosemide (7.5 mg kg⁻¹) and the combination of furosemide (7.5 mg kg⁻¹) and nalfurafine (5 μg kg⁻¹) over 90-min in conscious rats. As expected i.v. furosemide produced a significant increase in urine flow rate, urinary sodium excretion, and urinary potassium excretion when compared to respective control values. The peak increases in each of these renal excretory parameters was observed 10-min after drug injection and levels remained significantly elevated above respective control for 30 min. Urinary potassium excretion decreased below control values at 80 and 90 min. Furosemide did not produce any change in MAP or HR throughout the protocol.

When furosemide and nalfurafine were co-administered as an i.v. injection, a notable difference occurred in the pattern of renal excretory responses. In this regard, urine flow rat again significantly increased immediately following injection, but the levels remained significantly elevated above control and saline-treated rats at the 50, 60, and 70-min time points (FIG. 3A). Further, total urine output was significantly greater in rats treated with the combination of furosemide and nalfurafine as compared to furosemide alone (FIG. 3B: 11.80±0.20 mL vs 9.20±0.75 mL, respectively). The co-administration of furosemide and nalfurafine also initially increased urinary sodium and potassium excretion, but the duration of these responses was shortened with values returning to pre-drug control levels by the 20-and 30-min time points, respectively. As illustrated (FIG. 1A), in rats treated with furosemide and nalfurafine the levels of urinary sodium and potassium excretion were significantly lower over time periods 10-40-min as compared to rats that received furosemide alone. Interestingly, as compared to control levels, urinary potassium excretion was significantly decreased at the 50-, 60-, and 70-min time periods in rats that received furosemide and nalfurafine. Notably, the concentration of sodium and potassium and osmolality measured in the cumulative 90-min urine output of rats treated with furosemide and nalfurafine was markedly decreased as compared to levels measured in rats treated with furosemide alone (FIG. 3B: [Na+] 53.22±4.00 μeq ml⁻¹ vs 138±1.58 μeq ml⁻¹; [K+] 6.03±0.64 μeq ml⁻¹ vs 14.51±0.63 μeq ml⁻¹; Urine Osmolality, 181±13.16 mmol kg⁻¹ vs 379.8±15.33 mmol kg⁻¹, respectively). As compared to control levels, the combination treatment of furosemide and nalfurafine decreased MAP throughout the 90-min protocol (FIG. 3A; 40-min peak Δ; −11 mmHg) with levels also significantly different than furosemide alone over time points 20-90 min. Heart rate did not change in either treatment group throughout the study.

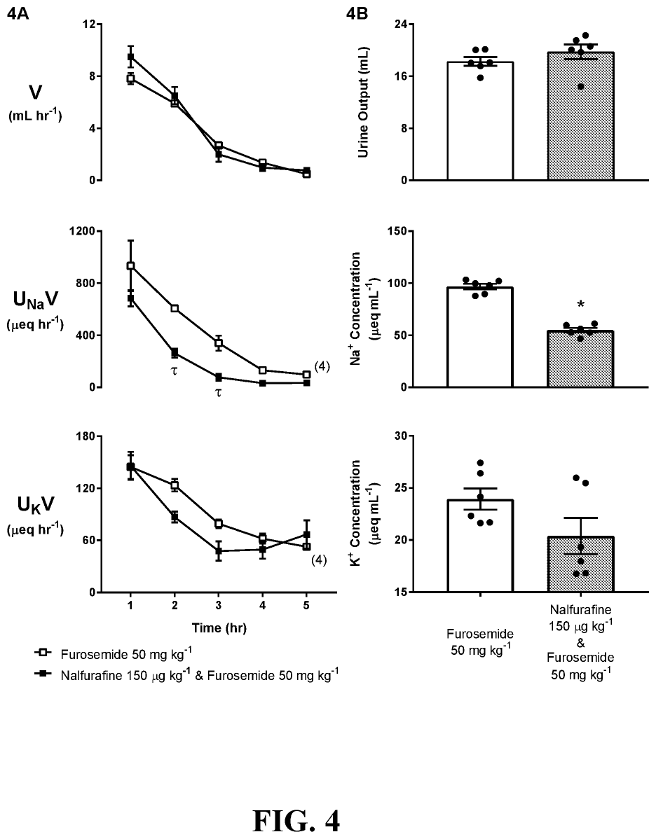

The renal excretory responses produced by the oral administration (gavage) of furosemide (50 mg kg⁻¹) and the combination of furosemide (50 mg kg⁻¹) and nalfurafine (150 μg kg⁻¹) are displayed in FIG. 4A and FIG. 4B. Combination treatment of these compounds did not produce any significant differences in urine output between treatment groups (FIG. 4A). However, the combination did notably decrease urinary sodium excretion at the 2- and 3-hour time periods when compared to furosemide alone. Urinary potassium excretion was also lower at the 2 and 3-hour time periods in rats treated with the combination of furosemide and nalfurafine, but this difference did not quite reach statistical significance. The concentration of sodium in the cumulative 5-hour urine that was voided in rats treated with furosemide and nalfurafine was significantly lower than furosemide alone (FIG. 4B: [Na+] 54.88±2.15 μeq ml⁻¹ vs 96.76±2.66 μeq ml⁻¹). While the concentration of potassium in the urine collected over 5 hours was reduced in rats that received the combination treatment, this difference did not achieve statistical significance from rats that only received furosemide (FIG. 4A and FIG. 4B: [K+] 19.76±1.14 mL vs 18.28±0.67 mL; 20.39±1.73 μeq ml⁻¹ vs 23.94±1.02 μeq ml⁻¹, respectively).

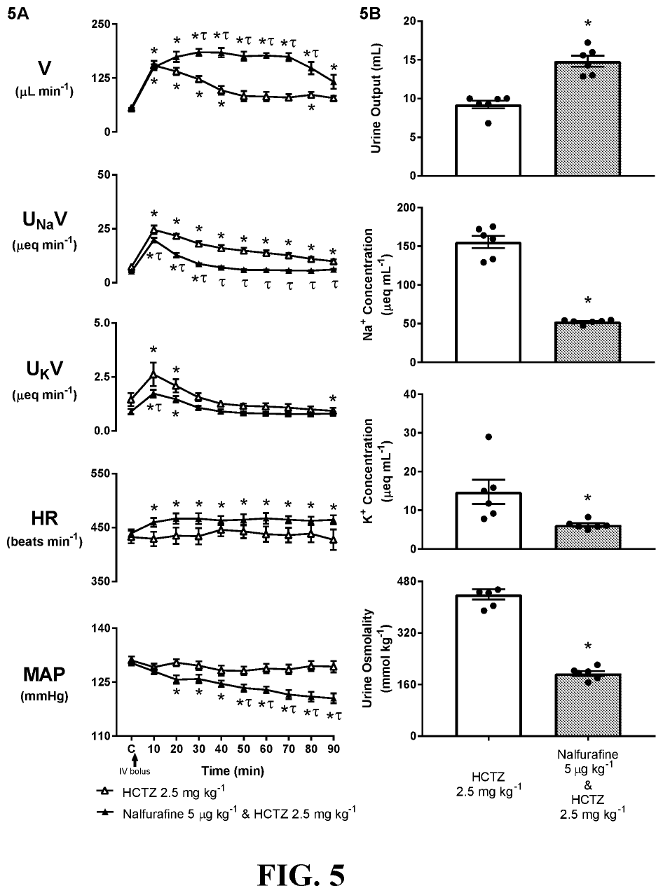

Cardiovascular and Renal Excretory Responses to Co-Administration of Hydrochlorothiazide and Nalfurafine: The changes in cardiovascular and renal excretory function produced by the i.v. administration of hydrochlorothiazide (HCTZ; 2.5 mg kg⁻¹) and the combination of HCTZ (2.5 mg kg⁻¹) and nalfurafine (5 μg kg⁻¹) in conscious rats are displayed in FIG. 5A. HCTZ significantly increased urine flow rate compared to the control value immediately after injection and returned to baseline levels by 50-min. In contrast, the combination of HCTZ and nalfurafine produced a marked increase in urine flow rate that remained elevated throughout the entire 90-min protocol. When compared to the HCTZ group, urine flow rate was significantly elevated in the HCTZ and nalfurafine treated animals from 30 to 80-min. The total cumulative urine output produced by the combination of drugs was significantly higher than HCTZ alone (FIG. 5B: 14.83±0.71 mL vs 9.22±0.50 mL, respectively). As shown in FIG. 5A, HCTZ also evoked a marked increase in urinary sodium excretion which was significantly elevated above the respective control level throughout the 10-90 min experimental periods. In contrast, in animals treated with HCTZ and nalfurafine, the natriuretic response was markedly shorter in duration (30-min) and of lower magnitude at each time point as compared to levels attained in the HCTZ group. The concentration of sodium in the cumulative 90-min urine output was significantly lower in the HCTZ and nalfurafine treated group as compared to the HCTZ alone group (FIG. 5B: [Na+] 52.54±1.06 μeq ml⁻¹ vs 155.6±8.04 μeq ml⁻¹, respectively). Both HCTZ and the combination drug treatment evoked an increase in urinary potassium excretion compared to respective control values. However, the magnitude of the kaliuresis was significantly lower in the HCTZ and nalfurafine group (FIG. 5A). Similarly, the concentration of potassium in the cumulative 90-min urine output was significantly lower in the combination drug treatment group as compared to HCTZ alone (FIG. 5B: [K+] 6.23±0.45 μeq ml⁻¹ vs 14.76±3.12 μeq ml⁻¹, respectively). Additionally, when compared to HCTZ alone, the combination of nalfurafine and HCTZ significantly decreased urine osmolality (FIG. 5B: 194.2±7.80 mmol kg⁻¹ vs 439.3±15.94 mmol kg⁻¹). In these studies, i.v. HCTZ alone did not produce any changes in MAP or HR. This contrasts with the combination of HCTZ and nalfurafine which produced a slight but significant decrease in MAP (FIG. 5A; 90-min peak Δ; −10 mmHg) which was statistically different than levels in the HCTZ group over 50-90 min periods. The combination of HCTZ and nalfurafine elicited a slight but significant increase in HR when compared to the respective control value throughout the protocol.

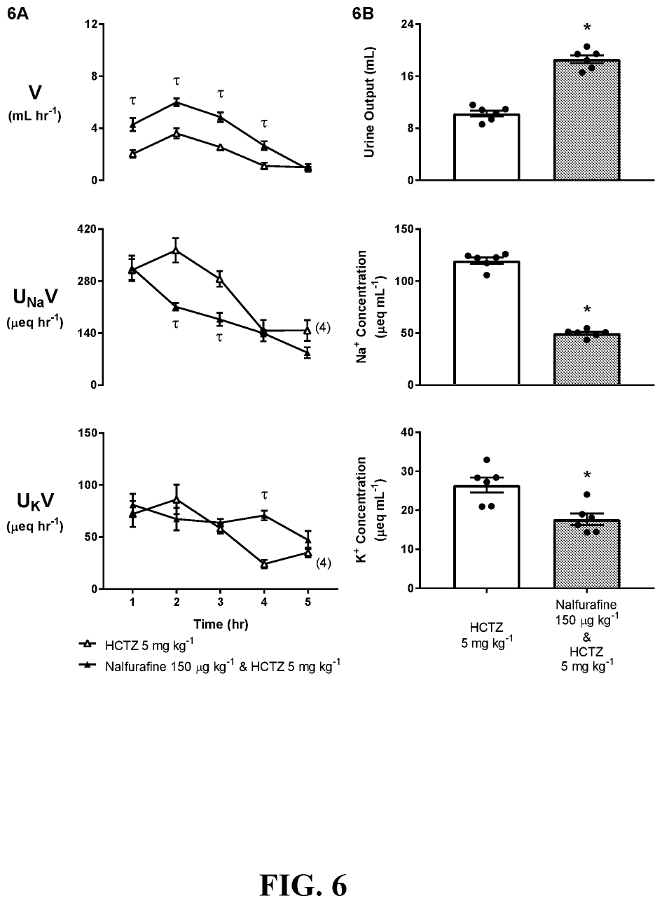

FIG. 6A illustrates the changes in renal excretory function produced by the oral administration (gavage) of HCTZ (5 mg kg⁻¹) and the combination of HCTZ (5 mg kg⁻¹) and nalfurafine (150 μg kg⁻¹). As shown, combination drug treatment significantly increased urine flow rate to a greater level than that produced by HCTZ (5 mg kg⁻¹) alone at the 1, 2, 3 and 4-hr periods (FIG. 6A and FIG. 6B). Thus, the cumulative amount of urine produced by the combination was notably higher than HCTZ alone (FIG. 6B: 18.62±0.61 mL vs 14.55±0.57 mL, respectively). The combination of HCTZ and nalfurafine also produced a markedly blunted natriuretic response as compared to HCTZ alone (FIG. 6A, periods 2 and 3-hrs). Of merit, despite the overall increase in urine output produced by HCTZ and nalfurafine, this combination drug treatment produced a significant decrease in the cumulative urine sodium concentration as compared to HCTZ (FIG. 6B: [Na+] 49.80±1.51 μeq ml⁻¹ vs 119.70±3.04 μeq ml⁻¹, respectively). Oral HCTZ and nalfurafine also decreased the total potassium concentration as compared to HCTZ alone (FIG. 6B: [K+] 17.69±1.48 μeq ml⁻¹ vs 26.50±1.91 μeq ml⁻¹).

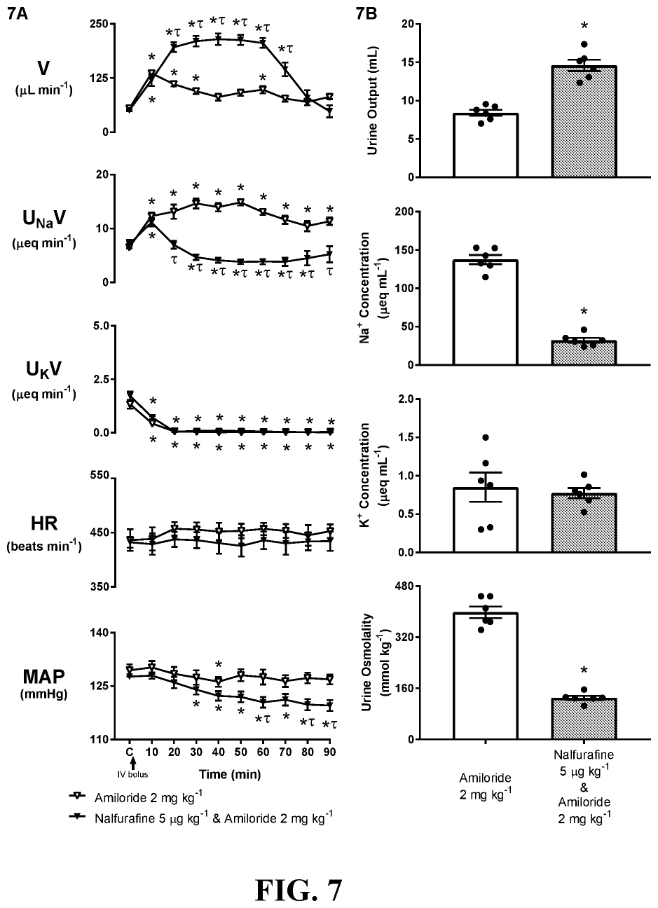

Cardiovascular and Renal Excretory Responses to Co-Administration of Amiloride and Nalfurafine: The cardiovascular and renal excretory responses produced by i.v. administration of amiloride (2 mg kg⁻¹) and amiloride (2 mg kg⁻¹) combined with nalfurafine (5 μg kg⁻¹) are shown in FIG. 7A. Amiloride alone produced a significant increase in urine flow rate above control levels that peaked at 10 min and lasted for 30 min. In contrast, the combination of amiloride and nalfurafine produced a marked diuresis that was greatly elevated above the respective group control level and levels attained in animals treated only with amiloride. Of merit, the enhanced increase in urine flow rate evoked by amiloride and nalfurafine persisted for 70-min before returning to baseline levels. The augmented diuretic response produced by the drug combination is also depicted in FIG. 7B, which shows that the cumulative urine excreted over the 90-min protocol was notably higher than that produced by amiloride alone (14.60±0.74 mL vs 8.42±0.40 mL, respectively). Amiloride also induced a marked increase in urinary sodium excretion that remained elevated throughout the entire protocol. This sustained natriuresis contrasts with the very brief (10-min) increase in urinary sodium excretion produced by the combination of amiloride and nalfurafine, which over periods 30-80-min then converted to a marked antinatriuresis. As depicted in FIG. 7B, despite the pronounced diuresis, the concentration of sodium in the cumulative urine excreted over the 90-min protocol was substantially lower in the amiloride and nalfurafine group as compared to that measured in the rats treated only with amiloride ([Na+] 32.32±3.25 μeq ml⁻¹ vs 137.60±6.01 μeq ml⁻¹, respectively). Both amiloride alone and the combination produced a profound antikaliuresis that was significantly different than respective group control values throughout the entire protocol (FIG. 7A). However, there were no differences in the concentration of potassium in the cumulative urine voided between the combined and amiloride treated groups (FIG. 7B: [K+] 0.77±0.07 μeq ml⁻¹ vs 0.85±0.19 μeq ml⁻¹, respectively). Additionally, the combination of nalfurafine and amiloride significantly decreased urine osmolality as compared to amiloride alone, (130±6.61 mmol kg⁻¹ vs 398.3±18.05 mmol kg⁻¹, respectively). Whereas amiloride had minimal effects on MAP, the drug combination significantly decreased MAP at 30-min which lasted throughout the protocol (FIG. 7A; 90-min peak Δ; −8 mmHg). Heart rate did not change throughout the study in both treatment groups.

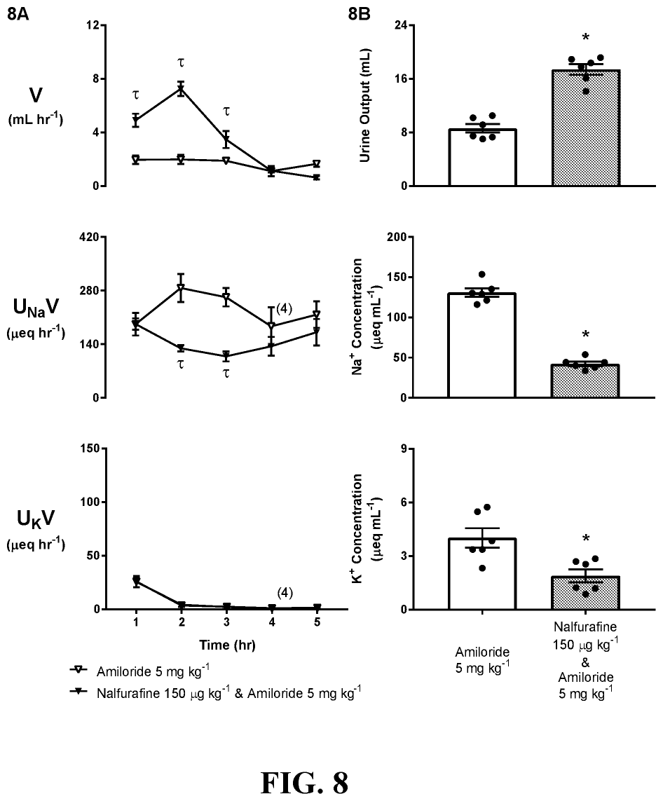

The time course effects of oral administration (gavage) of amiloride (5 mg kg⁻¹) and the combination of amiloride (5 mg kg⁻¹) and nalfurafine (150 μg kg⁻¹) on renal excretory function in rats placed in metabolic cages are displayed in FIG. 8A. The drug combination markedly increased urine flow rate when compared to amiloride alone at the 1, 2, and 3-hr time points. This is also depicted in FIG. 8B, which shows that the total cumulative urine voided over the 5-hr study was substantially greater in the amiloride and nalfurafine versus amiloride group (17.41±0.79 mL vs 8.61±0.63 mL, respectively). Additionally, while amiloride increased urinary sodium excretion, the combination of amiloride and nalfurafine notably decreased the renal excretion of sodium during the 2- and 3-hour time points after administration. As illustrated in FIG. 8B, the drug combination significantly decreased the sodium concentration in the cumulative urine excreted over the 5-hr period as compared to levels observed in the amiloride group (UNaV, 42.27±2.81 μeq ml⁻¹ vs 130.9±5.32 μeq ml⁻¹, respectively). In terms of potassium handling, the combination of amiloride and nalfurafine and amiloride alone excreted potassium at similar rates (FIG. 8A), but due to the enhanced urine output the combination decreased the total potassium concentration as compared to amiloride alone ([K+] 1.90±0.36 μeq ml⁻¹ vs 4.02±0.54 μeq ml⁻¹, respectively).

### Discussion

This study examined the cardiovascular and renal excretory responses produced by administration of the biased KOR agonist, nalfurafine, in conscious rats. The findings demonstrated that i.v. and oral administration of nalfurafine produced a profound increase in urine output and concurrent antinatriuresis and antikaliuresis. Moreover, nalfurafine markedly decreased the urinary concentration of sodium and potassium and urine osmolality, thus strongly indicating that the increase in urine output was mediated via inhibition of the synthesis/release of antidiuretic hormone. These findings are in agreement with prior studies in which nalfurafine was shown to evoke diuresis following subcutaneous injection (Inan, Lee et al., 2009) and extensive data showing that kappa opioids act centrally to produce diuresis by inhibiting antidiuretic hormone secretion (Leander, Zerbe, et al., 1985). Interestingly, i.v. nalfurafine also produced a slight, but significant (˜10 mmHg) decrease in MAP without inducing a concomitant increase in HR.

As an approach to investigate the nephron sites where nalfurafine acts to reabsorb sodium and potassium, we performed studies to examine whether the renal excretory responses to nalfurafine were altered by co-administration of diuretics that are known to selectively inhibit different tubular electrolyte transport pathways. The findings showed that nalfurafine was still able to increase renal tubular sodium and potassium reabsorption when combined with either a loop (furosemide), thiazide (hydrochlorothiazide), or potassium-sparing (amiloride) diuretic. This was demonstrated by the observation that in each case, the natriuresis and kaliuresis (thiazide and loop diuretics) produced by the standard-of-care diuretic was significantly decreased by co-administration of nalfurafine to respective levels at, or below, baseline control levels. These findings indicate that nalfurafine does not act via a transport channel located in a single nephron segment but instead, decreases the renal excretion of sodium and potassium by concurrently activating multiple tubular transport channels in different nephron segments. It should be noted that in these studies it was not determined whether nalfurafine affects the renal handling of these electrolytes via altering a transport pathway in the proximal convoluted tubule. This is of merit, since the proximal tubule is an active site in which both sodium and potassium are reabsorbed, which may, in part, account for the concurrent antinatriuretic and antikaliuretic responses produced by nalfurafine.

Regarding mechanisms, without wishing to be bound by theory, i.v. and oral nalfurafine activate renal nerve-dependent (central) and independent (peripheral) pathways that influence the renal handling of sodium and potentially potassium at multiple nephron segments. This is shown since central administration of KOR agonists are known to activate the renal sympathetic nerves and produce a marked decrease in urinary sodium excretion (potassium not studied), a response that is prevented by prior bilateral renal denervation (Kapusta & Obih, 1993). However, in other studies the antinatriuretic response produced by peripheral administration (i.v. infusion) of kappa opioids was not abolished by prior removal (renal denervation) of the renal sympathetic nerves (Kapusta, Jones, et al., 1989). In this latter case, the peripheral renal nerve-independent pathway may involve a direct renal action of nalfurafine to alter tubular transport processes and/or the secretion of circulating hormones such as renin, angiotensin II, aldosterone or catecholamines. Further studies are needed to validate the roles of these humoral and/or renal sympathetic nerve pathways in mediating the renal excretory response to nalfurafine.

Given nalfurafine's G-protein bias and reported lack of psychotomimetic effects, the water diuretic and blood pressure lowering effects of this compound are of merit when considering new approaches to treat patients with hypertension, cardiovascular disease, and volume overload. While this is of interest, it is well established that thiazide and loop diuretics have been used successfully for decades for the management of hypertension and removal of edema in heart failure. Despite their therapeutic efficacy, thiazides have been shown to increase the risk of hypokalemia and hyponatremia in the general population when used to treat hypertension (Rodenburg, Visser, et al., 2014; Krogager, et. al., 2020). In decompensated heart failure, furosemide paired with nitrates is first-line treatment to remove excess fluid and reduce afterload on the heart. Yet, treatment of patients with loop diuretics can lead to hypokalemia and with continued use, diuretic resistance. Diuretic resistance, which has been considered as a reduction or loss of natriuretic/diuretic response to loop diuretics when administered at a ceiling dose (Felker, Ellison, et al., 2020; Wilcox, Testani, et al., 2020), occurs in one out of three patients with chronic heart failure and is a major contributor of recurring hospitalization in these patients (Ravnan et al., 2002; Trullàs, Morales-Rull, et al., 2016). Similarly, physiological compensatory mechanisms that lead to enhanced sodium/water reabsorption (diuretic breaking) are well recognized to negate the beneficial diuretic and antihypertensive effects of chronic thiazide and loop diuretic therapy (Trullàs, Morales-Rull, et al., 2016; Wilcox, Testani, et al., 2020). Based on the understanding of the underlying mechanisms involved in diuretic adaptation and resistance, the addition of a thiazide diuretic has been shown to be effective in overcoming loop diuretic resistance by producing sequential nephron blockade and thus, diuretic synergy (Dormans, Gerlag, et al., 1998; Jentzer, DeWald, et al., 2010; Trullàs, Morales-Rull, et al., 2016; Wilcox, Testani, et al., 2020). Importantly, however, augmentation of urinary sodium excretion and urine output achieved by combination thiazide and loop diuretic therapy may occur at even a greater risk of inducing severe hypokalemia, hyponatremia, and worsening renal function (Rodenburg, Hoorn, et al., 2013; Rodenburg, Visser, et al., 2014; Krogager, Mortensen, et. al., 2020).

An alternative strategy that has not previously been considered to manage clinically diagnosed fluid overload in patients who require decongestion (e.g., chronic heart failure) is to administer a thiazide or loop diuretic together with a KOR agonist. This combination therapy would take advantage of two drugs acting by different mechanisms and sites of action to concurrently promote the renal excretion of water. Thus, while a thiazide or loop diuretic increases urine output secondarily to blockade of renal tubular sodium reabsorption within the kidneys, KOR agonists evoke diuresis by a central action to inhibit antidiuretic hormone secretion. This is of interest since in response to loss of fluid caused by a loop diuretic, a KOR agonist would prevent the compensatory increase in antidiuretic hormone secretion triggered by the volume depletion. Without wishing to be bound by theory, this combination of drugs can provide the beneficial effect of minimizing electrolyte disturbances, since loop/thiazides and KOR agonists have opposing actions (increased versus decreased, respectively) on sodium/potassium excretion. Along these lines, the results of the present study demonstrated that when combined with furosemide, HCTZ, or amiloride, nalfurafine increased total urine output and markedly limited the amount of sodium and potassium excreted by these diuretics. Because nalfurafine and these standard of care diuretics have opposing actions on sodium transport it was uncertain what impact this would have on urine output. However, it is now clear that these therapies work in a sub-additive fashion, significantly increasing the diuresis to these diuretics while decreasing the sodium/potassium excreted. These findings are important given the noticeable electrolyte disturbances that standard-of-care diuretics can cause. Also, it is important to note that when combined with these diuretics, nalfurafine still decreased MAP; this contrasts with administration of the diuretics alone which did not cause any change in MAP. This signifies that nalfurafine causes a change in MAP through a mechanism unrelated to the renal excretion of water. Traditional KOR agonists have been shown to act directly on the vasculature to cause vasorelaxation; however, this remains unexplored with nalfurafine (Guo, Zhang, et al., 2007).

These studies have demonstrated that nalfurafine is a potent and efficacious diuretic with antinatriuretic, antikaliuretic, and antihypertensive effects. When combined with multiple classes of traditional diuretics, nalfurafine increased the diuretic efficacy of these therapies and limits the loss of electrolytes through i.v. and oral routes of administration. Considering the findings presented here and the lack of central adverse effects associated with typical KOR agonists, nalfurafine is a new therapy for the treatment of cardiovascular and renal disease states associated with fluid overload that warrants future investigation.

### REFERENCES CITED IN THIS EXAMPLE

Beck, T. C., Reichel, C. M., Helke, K. L., Bhadsavle, S. S., & Dix, T. A. (2019). Non-addictive orally-active kappa opioid agonists for the treatment of peripheral pain in rats. European Journal of Pharmacology, 856.
Dormans, T. P. J., Gerlag, P. G. G., Russel, F. G. M., & Smits, P. (1998). Combination diuretic therapy in severe congestive heart failure. Drugs, 55(2), 165-172.
Ehrich, J. M., Messinger, D. I., Knakal, C. R., Kuhar, J. R., Schattauer, S. S., Bruchas, M. R., Zweifel, L. S., Kieffer, B. L., Phillips, P. E. M., & Chavkin, C. (2015). Kappa opioid receptor-induced aversion requires p38 MAPK activation in VTA dopamine neurons. Journal of Neuroscience, 35(37), 12917-12931.
Felker, G. M, Ellison, D. H., Mullens, W., Cox, Z. L, & Testani, J. M. (2020). Diuretic therapy for patients with heart failure: JACC State-of-the-art review. J. Am. Coll. Cardiology, 75(10), 1178-1195.
Guo, H. T., Zhang, R. H., Huang, L. Y., Li, J., Liu, Y. L., Bi, H., Zhang, Q. Y., Wang, Y. M., Sun, X., Ma, X. L., Cheng, L., Liu, J. C., Yu, S. Q., Yi, D. H., & Pei, J. M. (2007). Mechanisms involved in the hypotensive effect of a κ-opioid receptor agonist in hypertensive rats. Archives of Medical Research, 38(7), 723-729.
Inan, S., Lee, D. Y. W., Liu-Chen, L. Y., & Cowan, A. (2009). Comparison of the diuretic effects of chemically diverse kappa opioid agonists in rats: Nalfurafine, U50,488H, and salvinorin A. Naunyn-Schmiedeberg's Archives of Pharmacology, 379(3), 263-270.
Jentzer, J. C., DeWald, T. A., & Hernandez, A. F. (2010). Combination of loop diuretics with thiazide-type diuretics in heart failure. J. Am. Coll. Cardiology, 56(19), 1527-1534.
Kapelios, C. J. (2015). High furosemide dose has detrimental effects on survival of patients with stable heart failure. Hellenic Journal of Cardiology, 56, 154-159.
Kapusta, D. R., Jones, S. Y., & DiBona, G. F. (1989). Role of renal nerves in excretory responses to administration of kappa agonists in conscious spontaneously hypertensive rats. Journal of Pharmacology and Experimental Therapeutics, 251(1), 230-237.
Kapusta, D. R., & Obih, J. C. (1993). Central kappa opioid receptor-evoked changes in renal function in conscious rats: participation of renal nerves. Journal of Pharmacology and Experimental Therapeutics, 267(1).
Kaski, S. W., White, A. N., Gross, J. D., Trexler, K. R., Wix, K., Harland, A. A., . . . Setola, V. (2019). Preclinical testing of nalfurafine as an opioid-sparing djuvant that potentiates analgesia by the mu opioid receptor-targeting agonist morphine. Journal of Pharmacology and Experimental Therapeutics, 371(2), 487-499.
Krogager, M. L., Mortensen, R. N., Lund, P. E., Bøggild, H., Hansen, S. M., Kragholm, K., . . . Torp-Pedersen, C. (2020). Risk of Developing Hypokalemia in Patients with Hypertension Treated with Combination Antihypertensive Therapy. Hypertension, 75(4), 966-972.
Krowicki, Z. K., & Kapusta, D. R. (2011). Microinjection of Glycine into the Hypothalamic Paraventricular Nucleus Produces Diuresis, Natriuresis, and Inhibition of Central Sympathetic Outflow. Journal of Pharmacology and Experimental Therapeutics, 337(1), 247-255.
Kumagai, H., Ebata, T., Takamori, K., Muramatsu, T., Nakamoto, H., & Suzuki, H. (2010). Effect of a novel kappa-receptor agonist, nalfurafine hydrochloride, on severe itch in 337 haemodialysis patients: A Phase III, randomized, double-blind, placebo-controlled study. Nephrology Dialysis Transplantation, 25(4), 1251-1257.
Leander, J. D., Zerbe, R. L., & Hart, J. C. (1985). Diuresis and suppression of vasopressin by kappa opioids: Comparison with mu and delta opioids and clonidine. Journal of Pharmacology and Experimental Therapeutics, 234(2), 463-469.
Liu, L. J., Yu, J. J., & Xu, X. L. (2018). Kappa-opioid receptor agonist U50448h protects against renal ischemia-reperfusion injury in rats via activating the pi3k/akt signaling pathway. Acta Pharmacologica Sinica, 39(1), 97-106.
Pfeiffer, A., Brantl, V., Herz, A., & Emrich, H. M. (1986). Psychotomimesis mediated by κ opiate receptors. Science, 233(4765), 774-776.
Ravnan S. L., Ravnan, M. C., & Deedwania, P. C. (2002) pharmacothareapy in congestive heart failure: diuretic resistance and strategies to overcome resistance in patients with congestive heart failure. Congest. Heart Fail, 8, 80-85.
Rodenburg, E. M., Visser, L. E., Hoorn, E. J., Ruiter, R., Lous, J. J., Hofman, A., Uitterlinden, A. G., & Stricker, B. H. (2014). Thiazides and the risk of hypokalemia in the general population. Journal of Hypertension, 32(10), 2092-2097.
Schattauer, S. S., Kuhar, J. R., Song, A., & Chavkin, C. (2017). Nalfurafine is a G-protein biased agonist having significantly greater bias at the human than rodent form of the kappa opioid receptor. Cellular Signalling, 32, 59-65.
Trullàs J. C., Morales-Rull, J. L., Casado, J., Freitas Ramírez, A., Manzano, L., & Formiga, F.; CLOROTIC investigators. (2015). Rationale and design of the “Safety and efficacy of the combination of loop with thiazide-type diuretics in patients with decompensated heart failure (CLOROTIC) Trial:” A double-blind, randomized, placebo-controlled study to determine the effect of combined diuretic therapy (loop diuretics with thiazide-type diuretics) among patients with decompensated heart failure. J. Card. Fail. 22(7), 529-536.
Van Blijderveen, J. C., Straus, S. M., Rodenburg, E. M., Zietse, R., Stricker, B. H., Sturkenboom, M. C., & Verhamme, K. M. (2014). Risk of hyponatremia with diuretics: Chlorthalidone versus hydrochlorothiazide. The American Journal of Medicine, 127, 763-771.
Velat, I., Bušić, Ž., Jurić Paić, M., & Čulić, V. (2020). Furosemide and spironolactone doses and hyponatremia in patients with heart failure. BMC Pharmacology and Toxicology, 21(1), 57.
Wilcox, C. S., Testani, j. M., & Pitt, B. (2020). Pathophysiology of diuretic resistance and its implications for the management of chronic heart failure. Hypertension, 76(4), 1045-1054.

### Example 4—Difelikefalin, a Peripherally Restricted KOR (Kappa Opioid Receptor) Agonist, Produces Diuresis Through a Central KOR Pathway

### Abstract

Difelikefalin is a peripherally restricted kappa opioid receptor (KOR) agonist that was recently approved by the FDA for pruritis in dialysis patients. Here, we investigated the cardiovascular and renal responses to difelikefalin and using the KOR antagonist norbinaltorphimine (norBNI), examined whether difelikefalin causes its diuretic and electrolyte-sparing effects through a central or peripheral KOR pathway. Nalfurafine was also used to determine the effects of norBNI pretreatment on a KOR agonist that crosses the blood-brain barrier. We hypothesized that difelikefalin would produce different diuretic effects compared to nalfurafine, given that typical KOR agonists are known to employ central KORs to inhibit the release of vasopressin. Following catheterization and isotonic saline infusion, conscious Sprague-Dawley rats were pretreated with norBNI centrally via an intracerebroventricular (ICV) cannula or peripherally via an intravenous catheter 4-6 hours prior to difelikefalin or nalfurafine treatment, and urine output, heart rate and mean arterial pressure (MAP) were recorded for 90 minutes. Difelikefalin produced a significant increase in urine output, and notable decrease in sodium excretion, potassium excretion and MAP. Interestingly, ICV norBNI pretreatment attenuated the increase in urine output and decrease in MAP caused by difelikefalin and nalfurafine but did not inhibit the electrolyte effects. However, IV norBNI pretreatment diminished all responses to difelikefalin and nalfurafine. Thus, these findings demonstrate that difelikefalin and nalfurafine utilize central KOR pathways to elicit a diuresis and a decrease in MAP but reabsorb electrolytes through a peripheral KOR pathway, providing important insight into two clinically useful KOR agonists.

### Introduction

The kappa opioid receptor (KOR) is a G-protein coupled receptor that is activated by the endogenous dynorphin opioid peptides.¹ Agonists of the KOR have been shown to produce a variety of therapeutically beneficial effects, such as nonaddictive analgesia, organ protection from ischemia/reperfusion, and diuresis.²⁻⁴ However, the clinical utility of KOR agonists has been hindered by their dysphoric/psychotomimetic effects, which have been shown to be mediated by activation of central KORs and a downstream beta-arrestin signaling pathway.^{5.6}

To avoid producing the dysphoric/psychotomimetic adverse effects, two novel KOR agonists have been developed through a strategy involving G-protein-biased signaling and peripheral restriction. Regarding biased signaling, nalfurafine is a G-protein-biased KOR agonist that has been approved in Japan and shown to have efficacy and safety in treatment of uremic pruritis in hemodialysis patients.^{7.8} Further, following a successful phase 3 clinical trial, the FDA has recently approved the first selective KOR agonist in the US, difelikefalin, which is a peripherally restricted KOR agonist used for treatment-resistant pruritis in patients undergoing hemodialysis.⁹ Neither nalfurafine nor difelikefalin were shown to produce the typical dysphoric effects evoked by KOR agonists, demonstrating that biased signaling and peripheral restriction are sound strategies to improve the clinical utility of KOR agonists.

The renal excretory effects of KOR agonists are well studied and these compounds have been shown to produce a water diuresis (aquaresis), wherein an increase in urine output occurs concurrent with a reduction (or no change) in urinary sodium and potassium excretion, and a decrease in urine osmolality.⁴ A number of studies have demonstrated that KOR agonists produce diuresis by activating central KORs along the hypothalamic-pituitary axis, which leads to inhibition of the secretion of vasopressin (antidiuretic hormone) from the pituitary and thus, a decrease in distal tubular water reabsorption.^{10,11} However, a few studies suggest that KOR-mediated diuresis is dependent upon a peripheral mechanism, since prior adrenal demedullation attenuated the water diuresis to KOR agonists in rats. ^{12,13} Given the recent advancements in clinical utility of nalfurafine and difelikefalin, it is important to understand how these drugs may affect cardiovascular and renal function and determine the central and/or peripheral site(s) of drug action. This is relevant since, in certain cases, the production of an increase in urine output (diuresis) may be considered as an adverse effect. Alternatively, the ability of a KOR agonist to increase urine output without producing dysphoria/psychotomimetic effects may be a breakthrough in the clinical development of water diuretics for treatment of patients with water retaining and/or hyponatremic states.

While the diuretic effects of nalfurafine have been characterized, the cardiovascular and renal excretory effects of difelikefalin remain unexplored.^{4.16} Therefore, the present studies were performed to investigate the cardiovascular and renal effects of difelikefalin in conscious Sprague-Dawley rats. Additionally, to delineate the central and peripheral effects and sites of action of difelikefalin and nalfurafine, studies using the KOR antagonist norBNI were performed. For these studies conscious Sprague-Dawley rats were pretreated with the KOR antagonist peripherally or centrally and the cardiovascular and renal responses to difelikefalin or nalfurafine were measured. These studies were conducted to test the overall hypothesis that difelikefalin and nalfurafine will produce different effects on urine output due to the varying ability of these drugs to cross the blood-brain-barrier.

### Methods

### Animals

Male Sprague-Dawley rats (Envigo Labs, Indianapolis, IN) were individually housed, allowed tap water and standard rodent diet ad libitum, and kept under a 12-hour light-dark cycle. Rats used in these experiments weighed between 300 and 350 grams and were randomly assigned to experimental groups. All procedures were conducted in accordance with the National Institutes of Health guidelines for the care and use of animals and approved by the Louisiana State University Health Sciences Center Institutional Animal Care and Use Committee.

### Surgical Procedures

Aseptic technique was adhered to for all surgeries and detailed surgical methods were followed as previously described.¹⁴ Briefly, 3-5 days before experimentation, rats requiring central ventricle access for administration of drug/vehicle into the brain were anesthetized with ketamine and xylazine and a stainless-steel cannula was stereotaxically implanted into the right lateral cerebral ventricle.¹⁴

On the morning of the experiment, rats were anesthetized and maintained with inhaled 2% isoflurane, and polyethylene catheters were surgically placed into the left femoral artery, left femoral vein, and urinary bladder.^{15.16} Following surgery, rats were placed in an elevated restrainer for recovery and collection of urine from an indwelling bladder catheter.¹⁵ After completion of experimental protocols, rats were anesthetized with isoflurane and decapitated.

### Acute Cardiovascular and Renal Excretory Studies

During stabilization of cardiovascular and renal excretory parameters, rats received an isotonic saline infusion (55 μl/min), which lasted throughout the duration of the experiment. To record mean arterial pressure (MAP) and heart rate (HR), arterial catheters were connected to a pressure transducer (MLT0670, ADInstruments, Sydney, Australia), which was connected to Powerlab 8/30 (ADInstruments) and visualized using LabChart software (v8.1.5; ADInstruments). After one hour of recovery from surgery, rats were pretreated with the KOR antagonist norBNI or isotonic saline vehicle through either an intracerebroventricular (ICV) catheter or intravenous (IV) catheter. Following demonstration of stabilization of cardiovascular and renal excretory function (˜4-6 hours), blood pressure and HR were measured, and urine was collected during two consecutive 10-minute control periods. Next, drug or vehicle (isotonic saline) was injected into the femoral vein and urine samples were collected every 10 minutes during a 90-minute experimental period. The following drug concentrations were used: nalfurafine 5 μg/kg, difelikefalin 10 μg/kg, norBNI 30 mg/kg (IV), norBNI 1 μg/5 μL (ICV). All groups are n=6. Urine sodium and potassium were measured using a flame photometer (model 943; Instrumentation Laboratories, Lexington, MA). Urine volume was measured gravimetrically, and urine osmolality was measured using a vapor pressure osmometer (model 5600; WESCOR, Logan, UT).

### Experimental Design and Analysis

All results are expressed as mean±SEM. For determining statistically different changes at time points within a treatment group, means were compared to the control value using 2-way ANOVA with repeated measures and a post hoc Dunnett test following confirmation of a significant F value. For statistically different changes at individual time points between treatment groups, mean values were compared using 2-way ANOVA with repeated measures and a post hoc Sidak test following confirmation of a significant/value. For means between groups for total urine output, urine sodium concentration, urine potassium concentration, and urine osmolality, a Student/test was used. Statistical significance was characterized as P<0.05 and statistics were performed on Prism 7.05 software (GraphPad Software, Inc, San Diego, CA).

### Materials

Difelikefalin was obtained from Med Chem Express, Monmouth Junction, NJ; nalfurafine and norBNI by MilliporeSigma, Burlington, MA.

### Results

### Acute Cardiovascular and Renal Responses to Difelikefalin

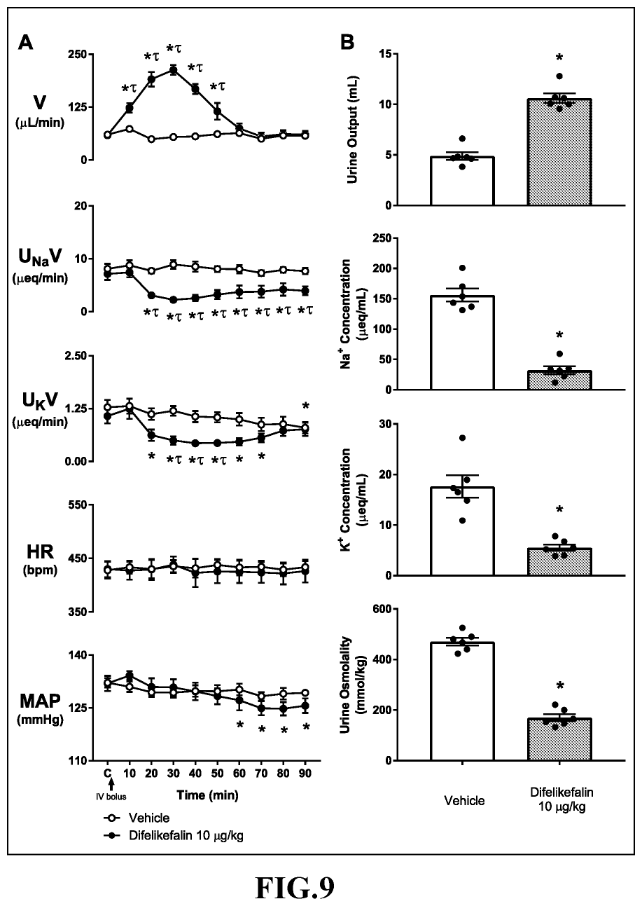

FIG. 9 displays the acute cardiovascular and renal responses to IV difelikefalin (10 μg/kg) or vehicle (isotonic saline) in conscious Sprague-Dawley rats. The dose for difelikefalin was chosen from previous dose-response studies. As shown, the IV bolus injection of isotonic saline vehicle did not produce any significant changes in renal excretory or cardiovascular function (FIG. 9A). In contrast, IV administration of difelikefalin produced a marked increase in urine flow rate when compared to respective group control value (C) and vehicle treated animals that began 10 minutes after drug administration and lasted for 50 minutes, with peak diuresis occurring at 30 minutes. Over the 90-minute experimental period, the difelikefalin treated group excreted significantly more total urine compared to the vehicle treated group (FIG. 9B: 10.62±0.47 mL versus 4.89±0.38 mL). Additionally, difelikefalin caused a significant decrease in urinary sodium excretion when compared to the group control value and vehicle treated animals that began at 20 minutes and lasted throughout the remainder of the protocol (FIG. 9A). Rats treated with difelikefalin also had a significantly lower urinary potassium excretion when compared to the group control value, which began at 20 minutes and lasted until 70 minutes post drug injection; these levels were also significantly different than urinary potassium excretion levels in the vehicle-treated group at 30, 40, and 50 minutes (FIG. 9A). The sodium concentration, potassium concentration, and urine osmolality of the total urine excreted in 90-minutes was significantly lower in the difelikefalin treated group compared to vehicle (FIG. 9B: [Na⁺] 32.22±6.48 μeq/mL versus 156.1±10.58 μeq/mL; [K⁺] 5.53±0.63 μeq/mL versus 17.64±2.23 μeq/mL; urine osmolality: 169.70±13.89 mmol/kg versus 470.8±14.88 mmol/kg). Difelikefalin elicited a significant decrease in blood pressure when compared to group control value that began at 60 minutes and lasted throughout the remainder of the protocol (FIG. 9A: 80-min peak Δ,−7 mmHg). Neither IV saline nor difelikefalin altered HR over the course of the protocol.

ICV norBNI modulates the Cardiovascular and Renal Responses to Difelikefalin and Nalfurafine

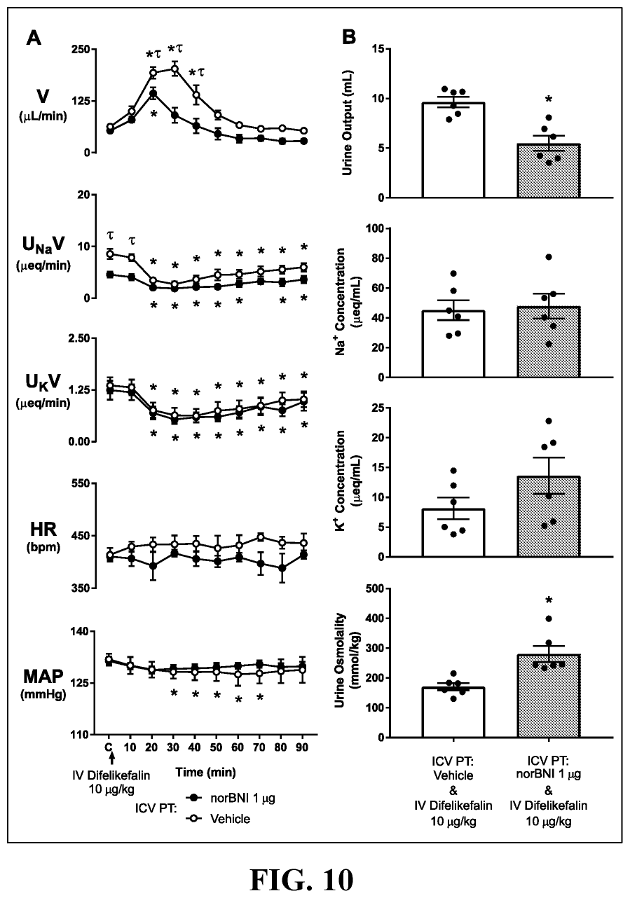

Changes in cardiovascular and renal excretory function produced by difelikefalin (10 μg/kg, IV) in rats pretreated centrally with the KOR antagonist norBNI (1 μg/5 μL, ICV) are displayed in FIG. 10. Rats that were pretreated ICV with vehicle demonstrated the typical and marked diuretic response produced by IV difelikefalin; urine flow rate was significantly increased compared to the group control value at 20, 30, and 40 min (FIG. 10A). In contrast, ICV norBNI pretreatment significantly attenuated the peak increase and duration of the difelikefalin-induced diuresis, with urine flow rate being higher than the control value only at the 20-minute time period (FIG. 10A); the levels for urine flow rate were also significantly lower than those in ICV vehicle pretreated rats at 20, 30, and 40 minutes. In rats pretreated ICV with norBNI, total urine output was significantly lower when compared to vehicle pretreatment (FIG. 10B: 5.49±0.75 mL versus 9.64±0.52 mL). Note that the total urine output produced by IV difelikefalin in the norBNI pre-treated rats (FIG. 10B: 5.49±0.75 mL) was not statistically different from the total 90-min urine output observed in rats that received IV vehicle treatment alone (see FIG. 9B: 4.89±0.38 mL). Interestingly, both ICV norBNI and ICV vehicle pretreatment groups displayed decreased urinary sodium and potassium excretion rates when compared to respective group control levels that began at 20 min and lasted throughout the protocol (FIG. 10A); both pretreatment groups also had similar urinary sodium and potassium concentrations, but rats pretreated ICV with norBNI had a significantly higher urine osmolality when compared to the vehicle pretreated group (FIG. 10B: [Na⁺] 47.92±8.30 μeq/mL versus 45.24±6.67 μeq/mL; [K⁺] 13.63±3.05 μeq/mL versus 8.16±1.81 μeq/mL; urine osmolality: 280±26.99 mmol/kg versus 170.7±12.04 mmol/kg). Also, in rats treated ICV with norBNI there was no significant change in MAP as compared to that seen in the vehicle pretreated group at 30-70 min when compared to baseline (FIG. 10A: 60-min peak Δ, −4 mmHg). No changes in HR were observed in either treatment group.

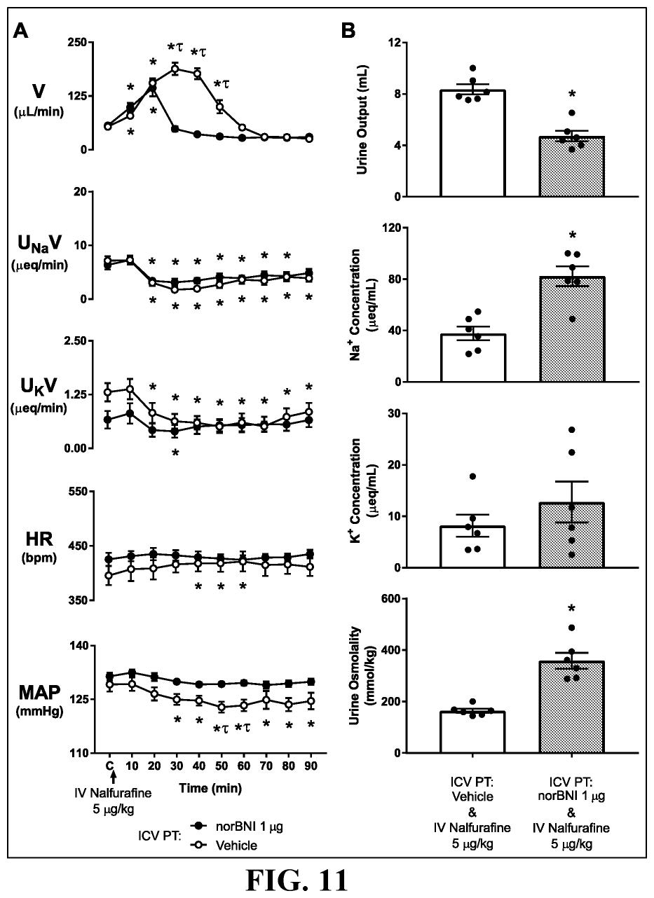

FIG. 11 displays the cardiovascular and renal excretory responses to nalfurafine (5 μg/kg, IV) following central pretreatment with norBNI (1 μg/5 μL, ICV) in conscious rats. As shown (FIG. 11A), in rats pretreated ICV with vehicle, IV nalfurafine produced a profound increase in urine flow rate (10-50 minutes), significant decrease in urinary sodium and potassium excretion (20-90 minutes), and decrease in MAP (30-90 minutes). However, in rats pretreated ICV with norBNI, nalfurafine only increased urine flow rate at the 10- and 20-minute periods and was significantly less in magnitude than levels observed in ICV vehicle pretreated rats at 30, 40, and 50 minutes. Also, rats pretreated ICV with norBNI excreted significantly less total urine in the 90-min protocol as compared to the level in ICV vehicle pretreated rats (FIG. 11B: 4.72±0.41 mL versus 8.37±0.40 mL). Importantly, ICV norBNI pretreatment did not prevent the nalfurafine-induced decrease in urinary sodium excretion (FIG. 11A); however, the concentration of sodium measured in the total 90-minute urine sample collected from central norBNI treated rats was significantly greater than the sodium concentration level observed in central vehicle pretreated rats (FIG. 11B: [Na⁺] 82.33±7.74 μeq/mL versus 37.78±5.35 μeq/mL). In contrast, in rats pretreated ICV with norBNI, nalfurafine did not change urinary potassium excretion as seen in the vehicle pretreated group, but the urine (90-min collection) potassium concentration was similar between the two groups (FIG. 11B: [K⁺] 12.77±3.99 μeq/mL versus 8.19±2.15 μeq/mL). Further, ICV norBNI prevented the nalfurafine-induced decrease in urine osmolality in the 90-minute urine sample (FIG. 11B: ICV norBNI group, 358.5±30.6 mmol/kg versus ICV vehicle group, 164±8.09 mmol/kg). Interestingly, the ICV pretreatment of rats with norBNI prevented the decrease in MAP to nalfurafine (FIG. 11A: ICV Vehicle group; 50-min peak Δ, −6 mmHg). A slight but statistically significant increase in HR was observed in rats pretreated with vehicle at the 40-, 50-, and 60-min periods.

IV norBNI Inhibits the Cardiovascular and Renal Responses to Difelikefalin and Nalfurafine

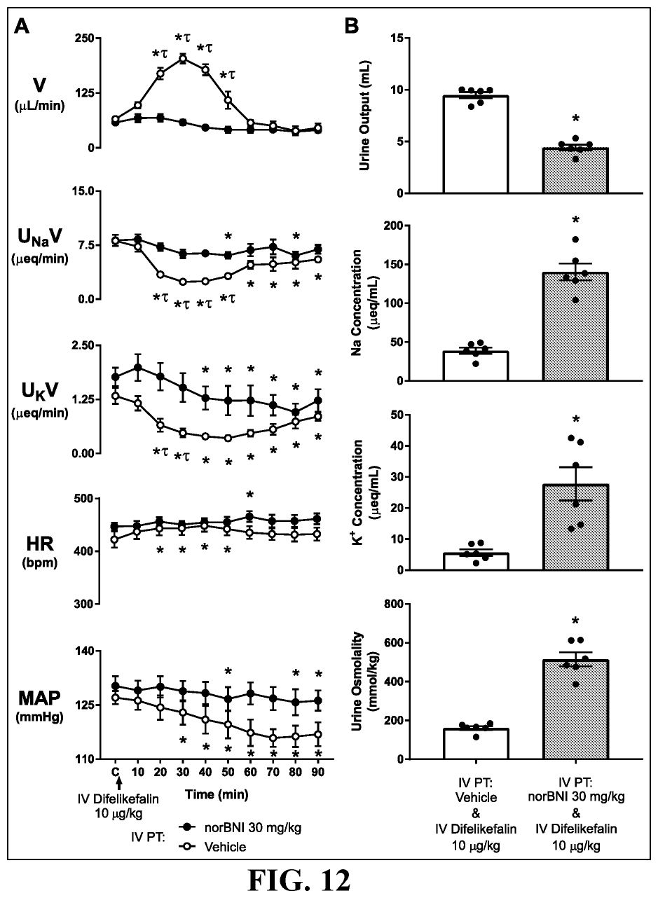

Studies were performed to examine the cardiovascular and renal responses to IV difelikefalin (10 μg/kg) in rats that were pretreated IV with the KOR antagonist norBNI (30 mg/kg); the findings of these studies are shown in FIG. 12. In vehicle-treated rats, difelikefalin produced an increase in urine flow rate beginning at 20 min and ending at 50 minutes post-drug administration. In contrast, IV norBNI pretreatment completely blocked the diuresis to IV difelikefalin (FIG. 12A). In accord with this finding, total urine output over the 90-min protocol was significantly less in the IV norBNI pretreated group compared to the vehicle pretreated group (FIG. 12B: 4.44±0.28 mL versus 9.49±0.30 mL). In vehicle treated rats, IV difelikefalin also produced a marked decrease in urinary sodium and potassium excretion (FIG. 12A). In contrast, the antinatriuresis produced by IV difelikefalin was blocked by IV norBNI pretreatment except at the 50- and 80-minute time periods. Similarly, IV norBNI pretreatment prevented the initial antikaliuresis (time periods 20-30 minutes) observed in vehicle-treated rats, although urinary potassium excretion was significantly decreased below the norBNI group control level thereafter for the remainder of the protocol (40-90 minutes). Notably, the sodium concentration, potassium concentration, and osmolality measured in the total 90-minute urine sample collected was significantly higher in the IV norBNI pretreated group when compared to the vehicle pretreated group (FIG. 12B: [Na⁺] 140.4±10.7 μeq/mL versus 38.93±4.01 μeq/mL; [K⁺] 27.78±5.35 μeq/mL versus 5.66±1.04 μeq/mL; urine osmolality: 515±35.8 mmol/kg versus 161.2±9.98 mmol/kg). Additionally, MAP was significantly decreased by IV difelikefalin in the vehicle pre-treatment group (FIG. 12A; vehicle group, 70-minute peak Δ, −11 mmHg), whereas the reduction in MAP was significantly attenuated by IV nor-BNI pre-treatment (FIG. 12A; norBNI group, 80-minute peak Δ, −4.5 mmHg). Both groups displayed slight but statistically significant increases in HR following IV difelikefalin.

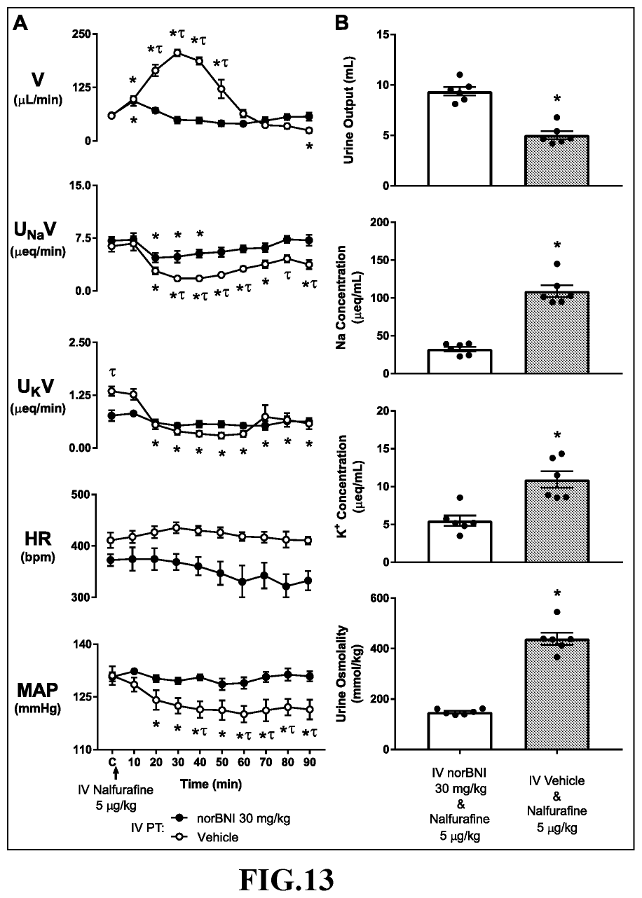

The acute cardiovascular and renal responses to IV nalfurafine (5 μg/kg) in rats pretreated IV with norBNI (30 mg/kg) are shown in FIG. 13. As shown, in vehicle pre-treated rats, IV nalfurafine produced a profound diuresis and concurrent decrease in urinary sodium and potassium excretion and decrease in MAP (FIG. 13A). However, in rats pretreated IV with norBNI, the diuresis to nalfurafine was nearly abolished, with a statistically significant increase in urine flow rate above group control levels occurring only at the 10-minute time point. This was also reflected in the total urine output, where IV norBNI pre-treated rats excreted significantly less urine over the 90-minute protocol than rats pretreated with vehicle (FIG. 13B: norBNI pre-treatment, 5.03±0.39 mL versus vehicle pre-treatment, 9.37±0.42 mL). While IV nalfurafine did produce a statistically significant decrease in urinary sodium excretion (20-40 minutes) in rats pre-treated IV with norBNI, the duration and magnitude of antinatriuresis was significantly less than that compared to the responses observed in vehicle-treated rats (FIG. 13A). Regarding potassium handling, IV norBNI pre-treatment prevented the marked antikaliuretic effect produced by IV nalfurafine. Note, however, that the control level for urinary potassium excretion was significantly lower in rats pre-treated with norBNI as compared to control levels in the vehicle pre-treated rats (FIG. 13A). The sodium concentration, potassium concentration, and urine osmolality measured in the total 90-minute urine sample in rats pretreated IV with norBNI was significantly higher than rats pretreated with vehicle (FIG. 13B: [Na⁺] 109.1±7.85 μeq/mL versus 32.56±3.05 μeq/mL; [K⁺] 10.94±1.09 μeq/mL versus 5.49±0.68 μeq/mL; urine osmolality: 439.2±24.02 mmol/kg versus 148.7±4.4 mmol/kg). Additionally, the decrease in MAP produced by IV nalfurafine (FIG. 13A: 60-min peak Δ−11 mmHg) was abolished in rats pre-treated IV with norBNI. IV nalfurafine did not produce any change in HR in either treatment group.

### Discussion

The present study examined the cardiovascular and renal excretory responses to difelikefalin, a peripherally restricted KOR agonist. A major finding of these investigations was demonstration that IV administration of difelikefalin produced a marked increase in urine output, a reduction in urinary sodium and potassium excretion, and a slight decrease in MAP in conscious rats. This is the first time that the cardiovascular and renal excretory effects of difelikefalin have been reported. The observation that difelikefalin produced a marked water diuresis is in congruence with several studies which have shown that KOR agonists evoke an increase in urine output associated with either no change or a decrease in urinary sodium excretion.^{4,16} However, previous KOR agonists which have been studied are known to readily cross the blood-brain barrier and mediate their diuretic actions through a central pathway involving inhibition of vasopressin secretion into the systemic circulation.¹⁰ In addition, KOR agonists are well recognized to produce dysphoria/psychotomimetic effects via a central action. Considering this knowledge and that difelikefalin is reported to be a peripherally restricted KOR agonist which is devoid of centrally mediated dysphoria/psychotomimetic effects when administered chronically to patients, the finding that difelikefalin produced a marked water diuresis was unexpected.⁹

As noted, considerable findings provide evidence that KOR agonists elicit their influence on the renal handling of water via a central pathway involving inhibition of the release/secretion of vasopressin.^{10,11} Following systemic administration, KOR agonists can enter the brain and activate KORs that are expressed in the supraoptic and paraventricular nuclei, as well as along the magnocellular neurons that project from these nuclei to the pituitary.^{17,18} In this manner, stimulation of KORs at these central sites causes hyperpolarization of the magnocellular neurons involved in mitigating the release of vasopressin.¹⁹ However, it is also plausible that a component of the diuresis produced by peripherally administered KOR agonists may occur by the drug binding to KORs situated along neurons outside of the blood brain barrier. The blood brain barrier begins to breakdown along the hypothalamic-pituitary axis which allows for communication between the circulatory system and the pituitary, the latter of which also expresses KORs. Therefore, it is possible that following peripheral administration, circulating KOR agonists may increase urine output, at least in part, by acting at the level of the pituitary to inhibit the release of vasopressin. Alternatively, it has been suggested that there is a link between the adrenal medulla and KOR-induced diuresis.^{12,13}

To determine whether the diuresis produced by IV difelikefalin was mediated via a central and/or peripheral site of action, we examined the renal excretory responses produced by IV administration of difelikefalin in rats pretreated either centrally (ICV) or peripherally (IV) with the KOR antagonist, norBNI. The findings of these studies showed that ICV norBNI markedly decreased the time course and magnitude of the diuretic response to IV difelikefalin with only a slight but significant increase in urine output noted 20-minutes after drug administration. Further, the total (cumulative) volume of urine excreted in response to IV difelikefalin over the duration of the study in rats pretreated centrally with norBNI was much lower and highly comparable to that excreted in animals that received only IV administration of vehicle. These findings support the conclusion that the primary pathway by which IV difelikefalin produces diuresis is via activation of KORs in the brain, presumably by inhibiting the secretion of vasopressin. It should be noted, however, that in other studies presented here, IV pretreatment of rats with norBNI, a KOR antagonist that readily crosses the blood-brain-barrier, completely prevented the diuresis to IV difelikefalin. Thus, when taken together these results support the premise that a small contribution of the diuresis to IV difelikefalin is mediated by the action of the drug in the periphery. However, these studies did not investigate the mechanism(s) (e.g., role of pituitary, adrenal glands, renal tubular sodium/potassium transporters) by which difelikefalin acted in the periphery to influence the renal handling of water.

Interestingly and in contrast to urine output, the findings of these studies showed that the antinatriuresis and antikaliuresis produced by IV difelikefalin were prevented by peripheral (IV), but not central (ICV) norBNI. These findings support the premise that IV difelikefalin mediates a decrease in the renal excretion of sodium and potassium via an action of the drug within the periphery. While the antinatriuretic/antikaliuretic effects of KOR agonists have been well reported, the mechanism and site(s) of action are less understood. In previous studies, we have shown that central administration of KOR agonists in rats produces antinatriuresis through the activation of the renal sympathetic nerves, a response that is abolished by prior bilateral renal denervation.²⁰ These findings demonstrate that KOR agonists can act centrally via a renal nerve pathway to enhance the renal tubular reabsorption of sodium.²⁰ In contrast, in other studies the peripheral administration of a KOR agonist also caused antinatriuresis, but this response was not abolished by prior bilateral renal sympathetic nerve denervation.²¹ Together, these findings demonstrate that KOR agonists can act centrally and peripherally via a renal nerve-dependent and independent pathway, respectively. In the present studies, IV difelikefalin produced an antinatriuretic and antikaliuretic response that was prevented by IV, but not ICV administration of norBNI. These findings indicate that the decrease in urinary sodium/potassium excretion evoked by IV difelikefalin was mediated by activation of KORs in the periphery, although the role of the renal nerves in producing this response was not studied. Considering a renal nerve-independent pathway, one possible mechanism may be that difelikefalin alters the renal tubular reabsorption of sodium and potassium by an intrarenal pathway. This is suggested since in an in vitro study it was demonstrated that there is an interaction between KORs and the Na⁺/H⁺-exchanger regulatory factor-1/Ezrin-radixin-moesin-binding phosphoprotein-50 (NHERF-1/EBP50); activation of the KOR caused an increase in the activity of Na⁺/H⁺-exchanger 3 (NHE3).²² Given the presence of NHE3 in the proximal tubule of the nephron and its sodium (and subsequently potassium) reabsorbing characteristics, this transporter could be involved in mediating the effects of difelikefalin on urinary sodium and potassium excretion.

To further study the site(s) of action by which KOR agonists alter the renal excretion of water and electrolytes, these investigations also examined the renal excretory responses produced by IV administration of nalfurafine, a G-protein-biased KOR agonist that penetrates the blood-brain barrier.²³ Of merit and potentially due to biased signaling, nalfurafine is reported to be devoid of producing dysphoria/psychotomimetic effects when administered chronically to treat pruritis in patients on dialysis.⁸ Comparatively, IV nalfurafine produced a very similar pattern of changes in renal excretory function in rats as that produced by IV difelikefalin, as demonstrated by a profound increase in urine flow rate and decrease in urinary sodium and potassium excretion. Further, as observed with difelikefalin, the diuretic response to IV nalfurafine was markedly attenuated by the ICV pretreatment of rats with norBNI, but the antinatriuretic effect was unaltered. Also, IV norBNI pretreatment significantly attenuated the diuretic, antinatriuretic, and antikaliuretic responses to IV nalfurafine. These findings further support the premise that the KOR agonists difelikefalin and nalfurafine act primarily in the brain to evoke diuresis but the effects on renal tubular reabsorption of sodium/potassium are mediated by an action of the KOR agonist in the periphery.

In addition to changes in renal excretory function, the peripheral administration of difelikefalin and nalfurafine were shown to produce a slight, but significant decrease in MAP, but not HR. Additionally, both ICV and IV norBNI pretreatment prevented the decrease in MAP. Reports on the effects of KOR agonists on blood pressure have been mixed and depend on the dose and ability of the drug to cross the blood-brain-barrier. Using KOR agonists with differing abilities to cross the blood-brain-barrier, Shen and Ingenito demonstrated that KOR agonists have central hypotensive actions at low doses but at higher doses a combination of central and peripheral actions leads to KOR-mediated hypertension.²⁴ Further, chronic or acute hippocampal administration of KOR agonists in several strains of rats has been shown to reduce blood pressure, a response that is prevented by bi-hippocampal microinjection of norBNI.²⁵ Our findings are in line with these observations and suggest that at the doses tested, difelikefalin and nalfurafine use a central KOR pathway to decrease MAP. Further studies, however, are required to determine the actual mechanism(s) by which these KOR agonists act centrally to reduce blood pressure.

In conclusion, the findings of the present study demonstrate, for the first time, the cardiovascular and renal effects produced by difelikefalin, the first KOR-selective agonist to be approved by the FDA in the US. The findings of these studies demonstrated that peripheral (IV) administration of difelikefalin, as well as the KOR agonist nalfurafine, produced a profound increase in urine output in rodents that is mediated predominately by an action of the drug in the brain, most probably by inhibiting the secretion of vasopressin into the systemic circulation. In contrast, it was shown that the antinatriuresis and antikaliuresis produced by IV difelikefalin and nalfurafine are mediated by an action(s) of the KOR agonist in the periphery. Given their lack of central adverse effects (psychotomimesis/dysphoria) in human patients, difelikefalin and nalfurafine could serve as novel water diuretic therapies for management of heart failure and other pathological states associated with fluid overload and hyponatremia/hypokalemia.

### REFERENCES CITED IN THIS EXAMPLE

1. Chavkin C, James I F, Goldstein A. Dynorphin Is a Specific Endogenous Ligand of the κ Opioid Receptor. Science (80-). 1982; 215(4531):413-415. doi:10.1126/SCIENCE.6120570
2. Beck T C, Reichel C M, Helke K L, Bhadsavle S S, Dix TA. Non-addictive orally-active kappa opioid agonists for the treatment of peripheral pain in rats. Eur J Pharmacol. 2019; 856:172396. doi: 10.1016/j.ejphar.2019.05.025
3. Tong G, Zhang B, Zhou X, et al. Kappa-Opioid Agonist U50,488H-Mediated Protection Against Heart Failure Following Myocardial Ischemia/Reperfusion: Dual Roles of Heme Oxygenase-1. Cell Physiol Biochem. 2016; 39(6):2158-2172. doi: 10.1159/000447911
4. Inan S, Lee D Y W, Liu-Chen L Y, Cowan A. Comparison of the diuretic effects of chemically diverse kappa opioid agonists in rats: Nalfurafine, U50,488H, and salvinorin A. Naunyn Schmiedebergs Arch Pharmacol. 2009; 379(3):263-270. doi: 10.1007/s00210-008-0358-8
5. Pfeiffer A, Brantl V, Herz A, Emrich H M. Psychotomimesis mediated by κ opiate receptors. Science (80-). 1986; 233(4765):774-776. doi: 10.1126/science.3016896
6. Ehrich J M, Messinger D I, Knakal C R, et al. Kappa opioid receptor-induced aversion requires p38 MAPK activation in VTA dopamine neurons. J Neurosci. 2015; 35(37): 12917-12931. doi:10.1523/JNEUROSCI.2444-15.2015
7. Schattauer S S, Kuhar J R, Song A, Chavkin C. Nalfurafine is a G-protein biased agonist having significantly greater bias at the human than rodent form of the kappa opioid receptor. Cell Signal. 2017; 32:59-65. doi:10.1016/j.cellsig.2017.01.016
8. Kumagai H, Ebata T, Takamori K, Muramatsu T, Nakamoto H, Suzuki H. Effect of a novel kappa-receptor agonist, nalfurafine hydrochloride, on severe itch in 337 haemodialysis patients: A Phase III, randomized, double-blind, placebo-controlled study. Nephrol Dial Transplant. 2010; 25(4): 1251-1257. doi: 10.1093/ndt/gfp588
9. Fishbane S, Jamal A, Munera C, Wen W, Menzaghi F. A Phase 3 Trial of Difelikefalin in Hemodialysis Patients with Pruritus. N Engl J Med. 2020; 382(3):222-232. doi: 10.1056/NEJMoa1912770
10. Rossi N, Brooks D. kappa-Opioid agonist inhibition of osmotically induced AVP release: preferential action at hypothalamic sites. Am J Physiol. 1996; 270(2 Pt 1). doi:10.1152/AJPENDO.1996.270.2.E367
11. Leander J D, Zerbe R L, Hart J C. Diuresis and suppression of vasopressin by kappa opioids: comparison with mu and delta opioids and clonidine. J Pharmacol Exp Ther. 1985; 234(2).
12. Blackburn T P, Borkowski K R, Friend J, Rance M J. On the mechanisms of kappa-opioid-induced diuresis. Br J Pharmacol. 1986; 89(3):593-598. doi:10.1111/J.1476-5381.1986.TB11160.X
13. Borkowski K R. Studies on the adrenomedullary dependence of kappa-opioid agonist-induced diuresis in conscious rats. Br J Pharmacol. 1989; 98(4): 1151-1156. doi: 10.1111/J.1476-5381.1989.TB12659.X
14. Kapusta D R, Kenigs V A. Cardiovascular and renal responses produced by central orphanin FQ/nociceptin occur independent of renal nerves. Am J Physiol—Regul Integr Comp Physiol. 1999; 277(4 46-4). doi: 10.1152/AJPREGU.1999.277.4.R987/ASSET/IMAGES/LARGE/AREG71027004X.JPE G
15. Krowicki Z K, Kapusta D R. Microinjection of Glycine into the Hypothalamic Paraventricular Nucleus Produces Diuresis, Natriuresis, and Inhibition of Central Sympathetic Outflow. Published online 2011. doi: 10.1124/jpet. 110.175398
16. Meariman J K, Sutphen J C, Gao J, Kapusta D R. Nalfurafine, a G-Protein-Biased KOR (Kappa Opioid Receptor) Agonist, Enhances the Diuretic Response and Limits Electrolyte Losses to Standard-of-Care Diuretics. Hypertension. Published online Dec. 2, 2021. doi: 10.1161/hypertensionaha. 121.18503
17. Shuster S J, Riedl M, Li X, Vulchanova L, Elde R. The kappa opioid receptor and dynorphin co-localize in vasopressin magnocellular neurosecretory neurons in guinea-pig hypothalamus. Neuroscience. 2000; 96(2):373-383. doi: 10.1016/S0306-4522(99)00472-8
18. Smith M J, Wise P M. Localization of kappa opioid receptors in oxytocin magnocellular neurons in the paraventricular and supraoptic nuclei. Brain Res. 2001; 898(1): 162-165. doi: 10.1016/S0006-8993(01)02154-0
19. Brown C, Ghamari-Langroudi M, Leng G, Bourque C. Kappa-opioid receptor activation inhibits post-spike depolarizing after-potentials in rat supraoptic nucleus neurones in vitro. J Neuroendocrinol. 1999; 11(11): 825-828. doi: 10.1046/J. 1365-2826.1999.00419.X
20. Kapusta D R, Obih J C. Central kappa opioid receptor-evoked changes in renal function in conscious rats: participation of renal nerves. J Pharmacol Exp Ther. 1993; 267(1).
21. Kapusta D R, Jones S Y, DiBona G F. Role of renal nerves in excretory responses to administration of kappa agonists in conscious spontaneously hypertensive rats. J Pharmacol Exp Ther. 1989; 251(1):230-237.
22. Huang P, Steplock D, Weinman E J, et al. kappa Opioid receptor interacts with Na(+)/H(+)-exchanger regulatory factor-1/Ezrin-radixin-moesin-binding phosphoprotein-50 (NHERF-1/EBP50) to stimulate Na(+)/H(+) exchange independent of G(i)/G(o) proteins. J Biol Chem. 2004; 279(24):25002-25009. doi: 10.1074/JBC.M313366200
23. Ando A, Sasago S, Ohzone Y, Miyamoto Y. Drug-Drug Interactions of a Novel κ-Opioid Receptor Agonist, Nalfurafine Hydrochloride, Involving the P-Glycoprotein. Eur J Drug Metab Pharmacokinet. 2016; 41(5):549-558. doi: 10.1007/S13318-015-0286-1
24. Shen S, Ingenito A J. Kappa-opioid receptors behind the blood-brain barrier are involved in the anti-hypertensive effects of systemically administered kappa-agonists in the conscious spontaneously hypertensive rat. J Pharm Pharmacol. 1999; 51(11): 1251-1256. doi: 10.1211/0022357991776985
25. Wang J Q, Ingenito A J. Comparative effects of intrahippocampal injection of dynorphin A(1-8), dynorphin A(1-13), dynorphin A(1-17), U-50,488H, and dynorphin B on blood pressure and heart rate in spontaneously hypertensive and normotensive Wistar-Kyoto rats. Peptides. 1994; 15(1):125-132. doi:10.1016/0196-9781(94)90180-5

### Example 5—Difelikefalin Prevents Development of Diuretic Resistance to Furosemide

One of the major causes of diuretic resistance is thought to be an intrarenal adaptation wherein the distal tubules undergo hypertrophy to enhance sodium reabsorption in response to the furosemide-induced increase in sodium delivery to the distal tubule. However, upon diuretic-induced loss of sodium, and subsequently water (urine), compensatory mechanisms may trigger the stimulation of vasopressin secretion into the systemic circulation and thereby impair the increase in urine output to diuretics. Given the unique diuretic mechanism of kappa opioid agonists (KOA) to inhibit central release of vasopressin, studies were performed to determine whether administration of the KOA, difelikefalin, would prevent the diuretic resistance to daily administration of furosemide.

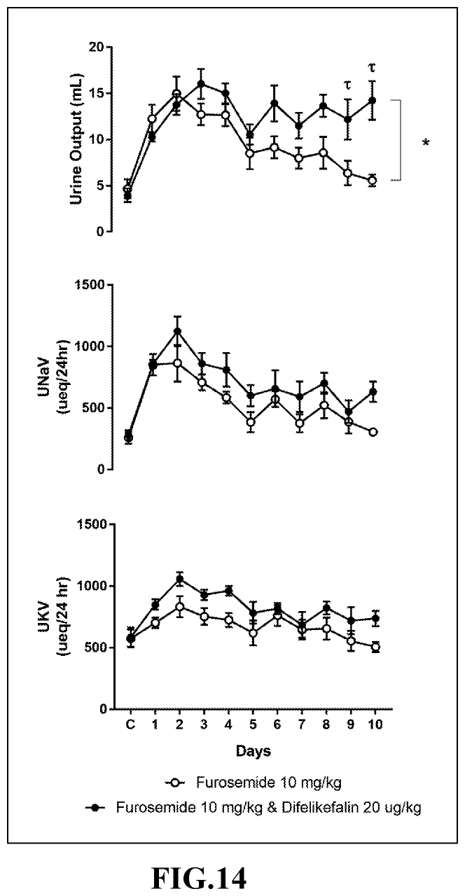

### FIG. 14 Methods

Male Sprague-Dawley rats were placed in individual metabolic cages in a room with 12-hour automatic light on/off control and allowed tap water and rodent chow ad libitum. Over the next two (2) days (control period), rats were administered vehicle (5% NaOH; 1 M PBS, IP) in the morning (9:00 am) and urine was then collected for 24-hours. Following the control period, furosemide alone (10 mg/kg) or furosemide in combination with difelikefalin (20 μg/kg) was administered IP in the morning once per day, and urine was collected for the next 24-hours; this was repeated for 10 consecutive days (experimental period)

### FIG. 14 Results and Significance

FIG. 14 displays the 24-hour renal excretory responses produced by furosemide alone and in combination with difelikefalin. In the furosemide alone group, peak diuresis occurred on day 2 (14.98±1.85 mL), whereas peak diuresis in the combination group occurred on day 3 (16.02±1.62 mL). Notably on day 10, the furosemide alone group excreted 5.57±0.63 mL of urine, while the combination group excreted 14.23±2.10 mL of urine. The combination of furosemide and difelikefalin excreted significantly more urine than the furosemide alone group on days 9 and 10, and the interaction between treatment groups was significantly different. Interestingly, there were no differences between groups regarding the excretion of sodium and potassium.

The data presented here demonstrate that repeated daily administration of furosemide leads to a reduction in the diuretic, natriuretic, and kaliuretic response or, in other words, diuretic resistance. However, when difelikefalin, a kappa opioid receptor agonist, is added to the furosemide regiment, the diuretic response remains elevated throughout the duration of the experimental protocol without excreting excessive sodium or potassium. This persistent elevation in urine output over the duration of the study signifies that difelikefalin prevents the diuretic resistance seen in the furosemide alone group.

### Example 6—Nalfurafine Prevents Development of Diuretic Resistance To Furosemide

Without wishing to be bound by theory, a component of the factors mediating diuretic resistance involves the secretion/release of vasopressin. Accordingly, studies were conducted to determine whether co-administration of the kappa opioid agonists (KOA), nalfurafine, would prevent the diuretic resistance to furosemide.

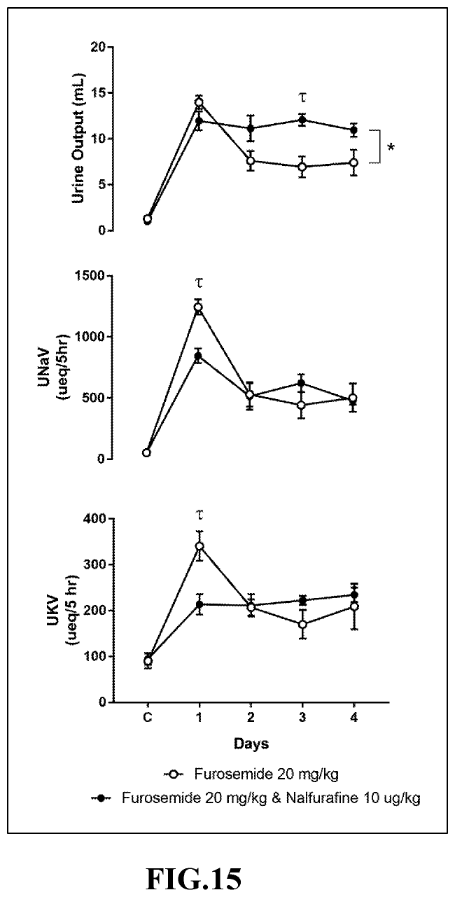

### FIG. 15 Methods

Male Sprague-Dawley rats were placed in individual metabolic cages in a room with 12-hour automatic light on/off control and allowed tap water and rodent chow ad libitum. Over the next two (2) days (control period), rats were administered vehicle (5% NaOH; 1 M PBS, IP) in the morning (9:00 am) and afternoon (2:00 pm) of each day, with urine collected for 5-hours starting immediately after the first injection. Following the control period, furosemide alone (20 mg/kg) or furosemide in combination with nalfurafine (10 μg/kg) was administered IP twice per day (9:00 am and 2:00 pm), with urine collected for 5-hours immediately after the first injection. This was repeated for 4 consecutive days (experimental period).

### FIG. 15 Results and Significance

FIG. 15 displays the 5-hour renal excretory responses produced by furosemide alone and in combination with nalfurafine. In the furosemide alone group, peak diuresis occurred on day 1 (13.99±0.72 mL), whereas peak diuresis in the combination group occurred on day 3 (12.07±0.64 mL). Notably on day 4, the furosemide alone group excreted 7.41±1.40 mL of urine, while the combination group excreted 10.96±0.72 mL of urine. The combination of furosemide and nalfurafine excreted significantly more urine than the furosemide alone group on day 3, and the interaction between treatment groups was significantly different. Additionally, the furosemide alone group excreted significantly more sodium and potassium than the combination group on day 1.

The data presented here demonstrate that repeated daily administration of furosemide leads to a reduction in the diuretic, natriuretic, and kaliuretic response or, in other words, diuretic resistance. However, when nalfurafine, a G-protein biased kappa opioid receptor agonist, is added to the furosemide regiment, the diuretic response remains elevated throughout the protocol without excreting excessive sodium or potassium. This persistent diuretic response signifies that nalfurafine prevents the diuretic resistance seen in the furosemide alone group.

### Example 7—Difelikefalin Reverses Diuretic Resistance to Furosemide Under Conditions of a Low Dietary Salt Intake

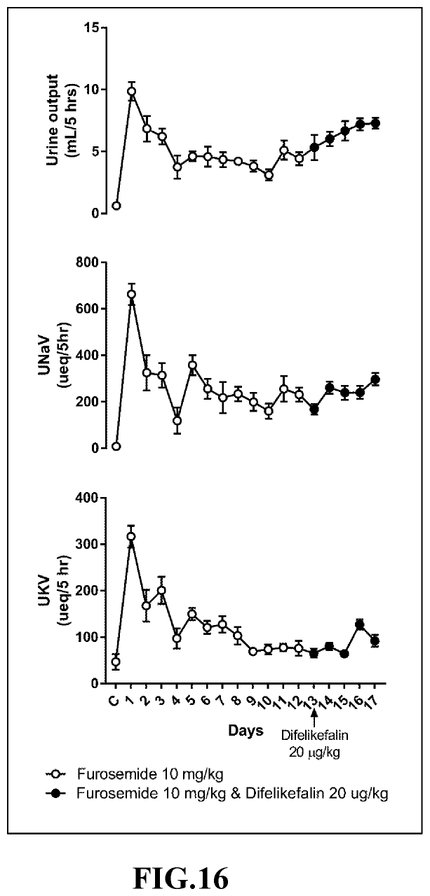

Without wishing to be bound by theory, a component of the factors mediating diuretic resistance is the secretion/release of vasopressin. Accordingly, studies were conducted to determine whether co-administration of the kappa opioid agonists (KOA), difelikefalin, would reverse established diuretic resistance to furosemide in rats consuming a low sodium diet. FIG. 16 Methods

Male Sprague-Dawley rats were placed in individual metabolic cages in a room with 12-hour automatic light on/off control and allowed tap water. For these studies, rats were placed on a low-salt diet (0.1% NaCl) for one week prior to and throughout the experimental protocol. A low-sodium diet was used as an approach to activate endogenous neural and humoral mechanisms that are actively involved in the renal retention of sodium; these pathways are also activated in sodium-retaining states such as heart failure, a pathology in which loop diuretics including furosemide are used but which diuretic resistance to loop diuretics including furosemide often develops. For the first two (2) days (control period), rats were administered vehicle (5% NaOH; 1 M PBS, IP) in the morning (9:00 am) and urine was then collected for 5-hours. Following the control period, furosemide alone (10 mg/kg) was administered IP once per day and urine was then collected for 5 hours; this was repeated for 17 days (experimental period). On day 13, furosemide was administered in combination with difelikefalin (20 μg/kg) for the remainder of the protocol (days 13-17).

### FIG. 16 Results and Significance

FIG. 16 displays the 5-hour renal excretory responses produced by daily IP administration of furosemide in rats placed on a low-sodium diet. Peak diuresis in response to furosemide alone occurred on day 1 (9.86±0.76 mL). On day 12, furosemide produced a diuresis of 4.43±0.54 mL thus demonstrating development of diuretic resistance with repeated furosemide dosing. However, following continued treatment of furosemide in combination with difelikefalin the urine output steadily increased, with the diuretic response on day 17 (7.28±0.43 mL) being significantly greater than that attained on day 12 (p<0.05; Student's t test). Interestingly, there were no differences between groups regarding the excretion of sodium and potassium.

The data presented here demonstrate that repeated daily administration of furosemide in rats placed on a low-sodium diet leads to a reduction in the diuretic, natriuretic, and kaliuretic response or, in other words, diuretic resistance. However, when the kappa opioid agonist difelikefalin was added to the furosemide regiment, the diuretic response increased to more than half of the peak diuretic response produced by furosemide alone. The increase in urine output produced by the addition of difelikefalin to the furosemide treatment regimen demonstrates that difelikefalin rescues the established diuretic resistance produced by repeated daily administration of furosemide.

### Example 8—Oral Administration of Nalfurafine Reverses Diuretic Resistance to Furosemide

Without wishing to be bound by theory, a component of the factors mediating diuretic resistance involves the secretion/release of vasopressin. Accordingly, studies were conducted to determine whether oral co-administration of the kappa opioid agonists (KOA), nalfurafine, would reverse established diuretic resistance to furosemide.

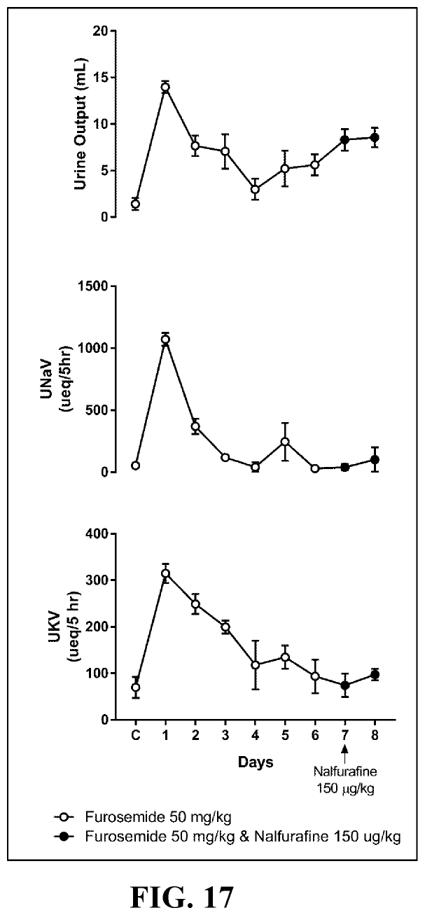

### FIG. 17 Methods

Male Sprague-Dawley rats were placed in individual metabolic cages in a room with 12-hour automatic light on/off control and allowed tap water and rodent chow ad libitum. Over the next two (2) days (control period), rats were administered vehicle (5% NaOH; 1 M PBS) orally (gavage) in the morning (9:00 am) and afternoon (2:00 pm) and urine was collected for 5-hours after the first oral treatment. Following the control period, furosemide alone (20 mg/kg) was administered orally twice per day (9:00 am and 2:00 pm), with urine collected for 5-hours immediately after the first oral dose. This was repeated for 8 consecutive days (experimental period). On day 7, furosemide was administered twice per day (oral) in combination with nalfurafine (150 μg/kg) for the remainder of the protocol.

### FIG. 17 Results and Significance

FIG. 17 shows the 5-hour renal excretory responses produced by oral administration of furosemide. Peak diuresis in response to furosemide alone occurred on day 1 (13.97±0.64 mL). On day 6, furosemide produced a diuresis of 5.61±1.14 mL indicating development of diuretic resistance; however, on day 8, the combination of furosemide and nalfurafine increased the diuretic response to 8.55±1.04 mL. Interestingly, there were no differences between groups regarding the excretion of sodium and potassium.

The data presented here demonstrate that repeated daily oral administration of furosemide leads to a reduction in the diuretic, natriuretic, and kaliuretic response or, in other words, diuretic resistance. However, when nalfurafine, a G-protein biased kappa opioid receptor agonist, is added to the furosemide regiment, the diuretic response increases to more than half of the peak diuretic response produced by furosemide alone. This increase in diuretic response following the addition of nalfurafine demonstrates that nalfurafine is able to treat or rescue the diuretic resistance seen following repeated oral administration of furosemide.

### Example 9—Nalfurafine Decreases Blood Pressure in Hypertensive Rats

### Introduction

Nalfurafine produces a clinically useful water diuresis and decrease in blood pressure. The studies presented in this example validated the chronic antihypertensive effects of nalfurafine in spontaneously hypertensive rats. These investigations examined whether nalfurafine would produce a decrease in blood pressure in this animal model of hypertension.

### Methods

### Animals

Male spontaneously hypertensive rats (SHR; Charles Rivers) were single housed and allowed tap water and standard rodent diet ad libitum. All procedures and experiments were conducted in accordance with the National Institutes of Health guidelines for the care and use of animals.

### Radiotelemetry Transmitter Implantation

Radiotelemetry transmitters (Data Sciences International, St. Paul, MN) were implanted into SHR for chronic recording of blood pressure and heart rate and followed as previously published.⁵⁶ Briefly, rats were anesthetized and maintained on 2% isoflurane and the right femoral artery was surgically exposed. The transmitter catheter was inserted into the femoral artery and advanced until reaching the abdominal aorta. The site exposing the femoral artery was sutured and rats were allowed 1 week of recovery. Telemetry data was analyzed using Ponemah software (DSI, St. Paul, MN).

### Experimental Protocols

Spontaneously hypertensive rats were orally administered (gavage) nalfurafine (0.5 mg/kg) or vehicle (water) once per day for 5 weeks following recovery from radiotelemetry transmitter insertion. Blood pressure was recorded once per week and the 12:00-14:00 time frame was plotted.

In a separate cohort, spontaneously hypertensive rats were administered a subcutaneous nalfurafine infusion (0.3 mg/day) or vehicle (isotonic saline) using an osmotic pump (2ML4; Alzet) for 4 weeks. For the insertion of the osmotic pumps, rats were anesthetized and maintained under 2% isoflurane, a small incision was made at the nape, and the osmotic pump was inserted between the scapulae. The incision was sutured, and rats were returned to their home cages. Blood pressure was recorded throughout the entirety of the experiment and the 20:00-22:00 time frame was plotted.

### Statistical Analysis

All results are expressed as mean±SEM. Statistically different changes for both experimental protocols were determined using 2-way ANOVA with repeated measures and were considered statistically different following a significant/value for an interaction between experimental group and time. Statistical significance was determined as P<0.05, and statistics were performed on Prism 7.05 software (GraphPad Software, Inc, San Diego, CA).

### Results

### Blood Pressure Effects of Chronic Oral Administration of Nalfurafine in Spontaneously Hypertensive Rats.

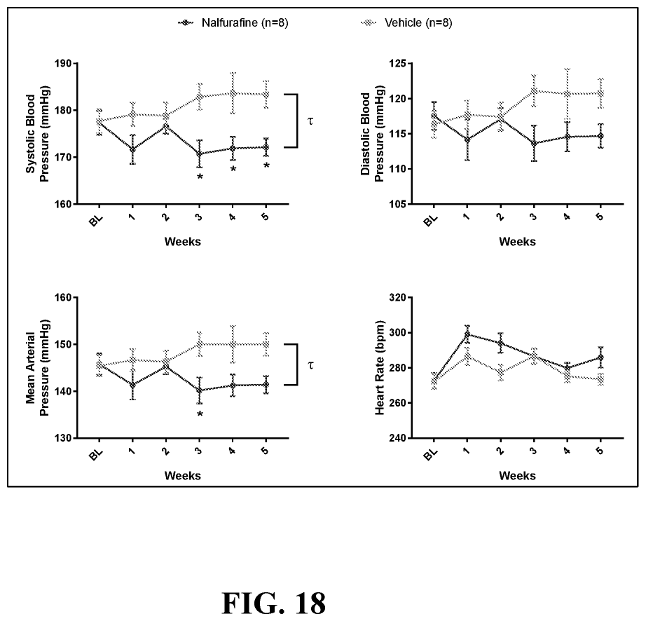

FIG. 18 displays the effects of oral administration of nalfurafine on blood pressure and heart rate in conscious spontaneously hypertensive rats. Over the course of 5 weeks, rats that were treated with vehicle (water) had a gradual increase in systolic blood pressure, diastolic blood pressure, and mean arterial pressure, with no change in heart rate. In contrast, rats that were treated with nalfurafine displayed a decrease in systolic blood pressure, mean arterial pressure, diastolic blood pressure. The interaction between the nalfurafine and vehicle treated groups were significantly different over time for systolic blood pressure, and systolic blood pressure was significantly lower in the nalfurafine group compared to vehicle at 3, 4, and 5 weeks. Additionally, the interaction between the nalfurafine and vehicle treated groups were significantly different over time for mean arterial pressure, with nalfurafine treated animals having a significantly lower mean arterial pressure at the 3-week time point. There was no significant interaction between treatment over time for diastolic blood pressure or heart rate.

### Changes in Blood Pressure Produced by Chronic Subcutaneous Infusion of Nalfurafine in Spontaneously Hypertensive Rats

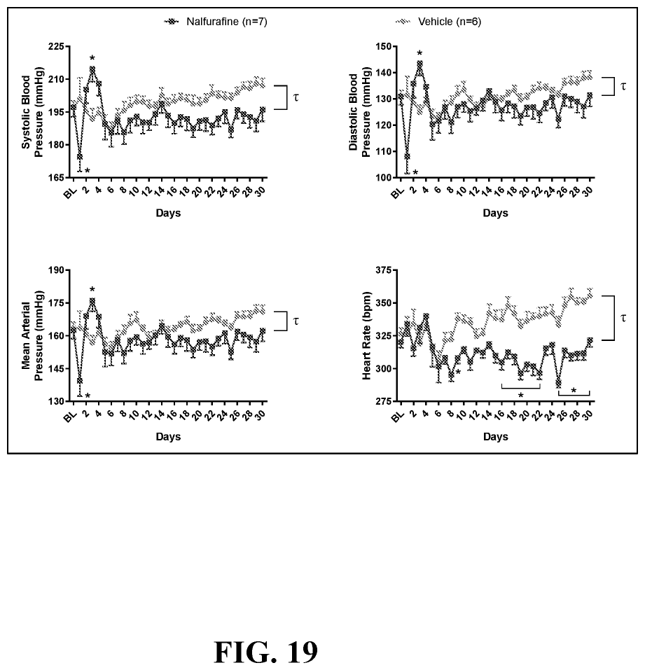

The changes in blood pressure and heart rate produced by chronic subcutaneous infusion of nalfurafine in conscious spontaneously hypertensive rats is shown in FIG. 19. Rats that were treated with vehicle (isotonic saline) displayed a gradual increase in systolic blood pressure, diastolic blood pressure, mean arterial pressure, and heart rate over the course of 30 days. However, rats that received a subcutaneous infusion of nalfurafine (0.3 mg/day) did not demonstrate an increase in these parameters. Additionally, the interaction between the nalfurafine and vehicle treated groups was significantly different over time for systolic blood pressure, diastolic blood pressure, mean arterial pressure, and heart rate. Rats treated with nalfurafine had a significantly different systolic blood pressure, diastolic blood pressure, and mean arterial pressure on day 1 and day 3 when compared to vehicle. Notably, the nalfurafine treated group had a significantly lower heart rate than the vehicle treated group on day 9, day 16-22, and day 25-30.

### Discussion

The studies presented in this example investigated the chronic antihypertensive effects of nalfurafine in spontaneously hypertensive rats through varying routes of administration. When administered orally for 5 weeks, nalfurafine produced a significant decrease in systolic blood pressure and mean arterial pressure when compared to animals that were administered vehicle. However, this effect was only seen acutely following administration of nalfurafine. Animals were dosed daily at 10:00, and the values presented in FIGS. 18 and 19 represent the 12:00-14:00 time frame. No other differences in blood pressure or heart rate were observed throughout the day. The results presented here demonstrate that tolerance does not develop to the acute effect on blood pressure produced by nalfurafine. Given the relatively short half-life of nalfurafine in rats (˜6 hours), a chronic effect on blood pressure was not observed. However, these findings led us to investigate the effects of a chronic infusion of nalfurafine on blood pressure.

When administered as a chronic subcutaneous infusion (mini-osmotic pump), nalfurafine elicited significant changes in systolic blood pressure, diastolic blood pressure, mean arterial pressure, and heart rate when compared to vehicle treatment. The values in FIGS. 18 and 19 represent the 20:00-22:00 time frame; given that rats are a nocturnal species, this time frame would occur during their “active” period. Notably, differences in blood pressure and heart rate between treatment groups were only observed during this active period. As depicted in FIGS. 18 and 19, spontaneously hypertensive rats treated with vehicle exhibited a steady increase over time in systolic blood pressure, diastolic blood pressure, mean arterial pressure, and heart rate. In contrast, rats that received nalfurafine did not demonstrate the temporal increase seen in vehicle treated rats, and blood pressure and heart rate values were significantly less than vehicle. Interestingly, 1 day of nalfurafine infusion resulted in a significant decrease (˜ 20 mmHg) in systolic, diastolic, and mean arterial pressure, highlighting the acute hypotensive effects of nalfurafine. However, this decrease was followed by 3 days of a rebound hypertensive response before leveling out on day 5.

While the effects on blood pressure are significant, the most prominent differences occurred in the heart rates between the nalfurafine and vehicle treated groups. Nalfurafine infusion produced a significantly decreased heart rate throughout the majority of the second half of the protocol when compared to vehicle. These findings are of merit given the known interaction between kappa opioid receptors and β-adrenergic receptors in the heart. In a previous study, the kappa opioid receptor agonist U50,488 was shown to inhibit the increase in [Ca2+]i transit in response to norepinephrine in isolated rat ventricular myocytes, and the effect was also inhibited by nor-binaltorphimine (norBNI), signifying that this inhibition is mediated through a kappa opioid receptor.⁵⁷ Given that the increase in [Ca2+]i transit in response to norepinephrine is mediated by β-adrenergic receptors, this study demonstrates that kappa opioid agonists can inhibit the stimulatory effects of β-adrenergic receptors in the heart. The findings presented here demonstrate that nalfurafine significantly reduces heart rate in hypertensive rats, which agrees with what was shown in isolated ventricular myocytes and elevates these findings to a broader physiological scale. In addition to the diuretic effects of nalfurafine, the ability to inhibit the effects of β-adrenergic receptors in the heart could further broaden the clinical potential of this compound in cardiovascular diseases.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method to reverse diuretic resistance, the method comprising administering to the subject an amount of a kappa opioid receptor agonist (KOA) sufficient to selectively increase urine output, wherein the KOA decreases or does not change urinary excretion of electrolytes.

2. The method of claim 1, wherein the subject suffers from diuretic resistance.

3. The method of claim 1, wherein the subject has failed to achieve diuresis with diuretic therapy prior to administering.

4. The method of claim 1, where the kappa opioid receptor agonist comprises an organic or synthetic small molecule or a peptide.

5. The method of claim 1, wherein the kappa opioid receptor agonist is a peripherally restricted KOA, a centrally acting KOA, or both.

6. The method of claim 1, wherein the electrolytes comprise sodium, potassium, chloride, or a combination thereof.

7. The method of claim 4, where the organic small molecule or peptide is JT09, nalfurafine, CR665, or difelikefalin.

8. The method of claim 4, where the organic or synthetic small molecule or peptide comprises a molecule of: 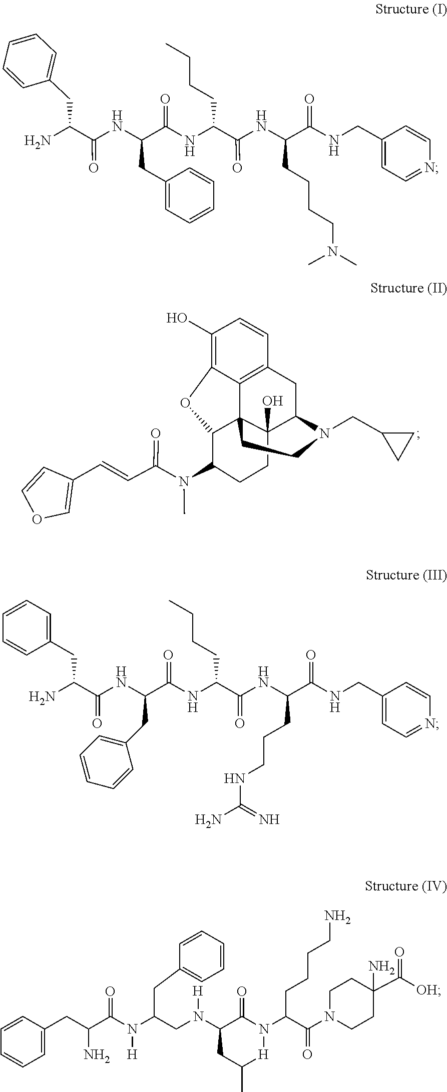
or any combination thereof.

9. The method of claim 1, further comprising administering to the subject one or more additional active agents.

10. The method of claim 9, wherein the one or more additional active agents comprises a diuretic.

11. The method of claim 10, wherein the diuretic comprises a loop diuretic, a thiazide or thiazide-like diuretic, or a potassium sparing diuretic.

12. The method of claim 11, wherein the loop diuretic comprises furosemide, torsemide, ethacrynic acid, or bumetanide.

13. The method of claim 11, wherein the thiazide or thiazide-like diuretic comprises hydrochlorothiazide, chlorthalidone, metolazone, indapamide, chlorothiazide, or bendroflurnethiazide.

14. The method of claim 11, wherein the potassium sparing diuretic comprises amiloride, triamterene, spironolactone, or eplerenone.

15. A method of preventing diuretic resistance, the method comprising administering to the subject an amount of a kappa opioid receptor agonist (KOA) sufficient to selectively increase urine output, wherein the KOA decreases or does not change urinary excretion of electrolytes, and the KOA is administered in combination with a diuretic.

16. A method of sensitizing a subject to a diuretic, the method comprising administering to the subject an amount of a kappa opioid receptor agonist (KOA) sufficient to selectively increase urine output, wherein the KOA decreases or does not change urinary excretion of electrolytes.

17. The method of claim 15 or claim 16, where the kappa opioid receptor agonist comprises an organic or synthetic small molecule or a peptide.

18. The method of claim 15 or claim 16, wherein the kappa opioid receptor agonist is a peripherally restricted KOA, a centrally acting KOA, or both.

19. The method of claim 15 or claim 16, wherein the electrolytes comprise sodium, potassium, chloride, or a combination thereof.

20. The method of claim 17, where the organic small molecule or peptide is JT09, nalfurafine, CR665, or difelikefalin.

21. The method of claim 17, where the organic or synthetic small molecule or peptide comprises a molecule of: 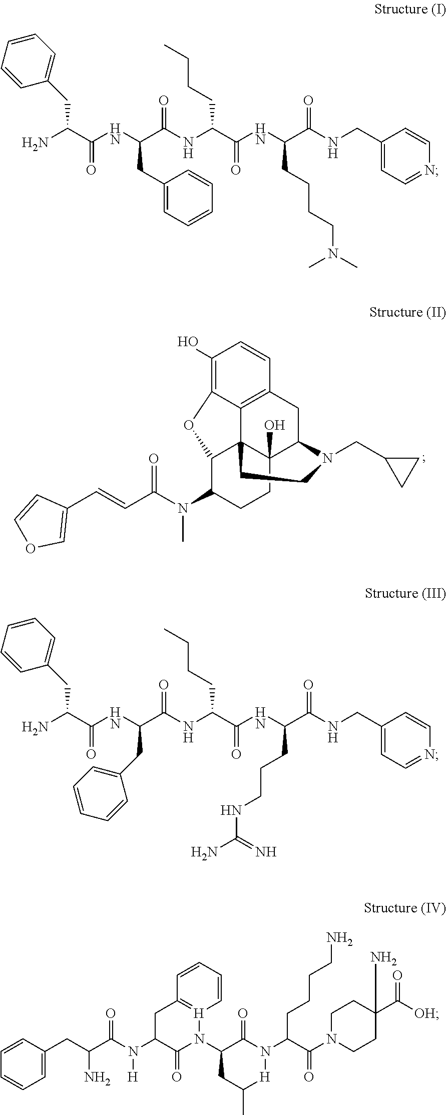
or any combination thereof.

22. The method of claim 15 or claim 16, further comprising administering to the subject one or more additional active agents.

23. The method of claim 22, wherein the one or more additional active agents comprises a diuretic.

24. The method of claim 23, wherein the diuretic comprises a loop diuretic, a thiazide or thiazide-like diuretic, or a potassium sparing diuretic.

25. The method of claim 24, wherein the loop diuretic comprises furosemide, torsemide, ethacrynic acid, or bumetanide.

26. The method of claim 24, wherein the thiazide or thiazide-like diuretic comprises hydrochlorothiazide, chlorthalidone, metolazone, indapamide, chlorothiazide, or bendroflurnethiazide.

27. The method of claim 24, wherein the potassium sparing diuretic comprises amiloride, triamterene, spironolactone, or eplerenone.

28. The method of claim 15 or claim 16, wherein the subject suffers from diuretic resistance.

29. The method of claim 15 or claim 16, wherein the subject has failed to achieve diuresis with diuretic therapy prior to administering.

30. A method of promoting urinary output, the method comprising administering to the subject an amount of a kappa opioid receptor agonist (KOA) sufficient to selectively increase urine output, wherein the KOA reduces or does not change urinary excretion of electrolytes.

31. The method of claim 30, where the kappa opioid receptor agonist comprises an organic or synthetic small molecule or a peptide.

32. The method of claim 30, wherein the kappa opioid receptor agonist is a peripherally restricted KOA, a centrally acting KOA, or a combination thereof.

33. The method of claim 30, wherein the electrolytes comprise sodium, potassium, chloride, or a combination thereof.

34. The method of claim 31, where the organic small molecule or peptide is JT09, nalfurafine, CR665, or difelikefalin.

35. The method of claim 31, where the organic or synthetic small molecule or peptide comprises a molecule of: 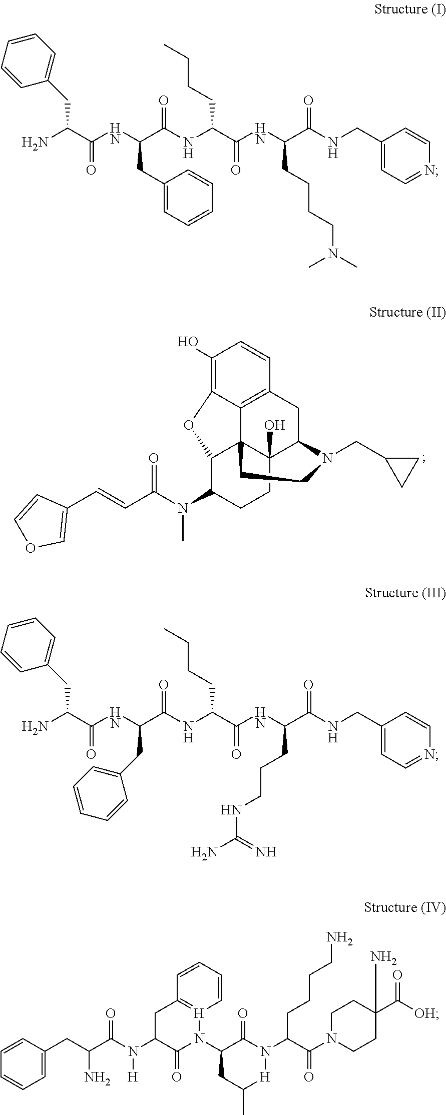
or any combination thereof.

36. The method of claim 30, further comprising administering to the subject one or more additional active agents.

37. The method of claim 36, wherein the one or more additional active agents comprises a diuretic.

38. The method of claim 37, wherein the diuretic comprises a loop diuretic, a thiazide or thiazide-like diuretic, or a potassium sparing diuretic.

39. The method of claim 38, wherein the loop diuretic comprises furosemide, torsemide, ethacrynic acid, or bumetanide.

40. The method of claim 38, wherein the thiazide or thiazide-like diuretic comprises hydrochlorothiazide, chlorthalidone, metolazone, indapamide, chlorothiazide, or bendroflurnethiazide.

41. The method of claim 38, wherein the potassium sparing diuretic comprises amiloride, triamterene, spironolactone, or eplerenone.

42. The method of claim 30, wherein the subject suffers from diuretic resistance.

43. The method of claim 30, wherein the subject has failed to achieve diuresis with diuretic therapy prior to administering.

44. A method of treating a subject afflicted with a cardiovascular disease, the method comprising administering to the subject an amount of a kappa opioid receptor agonist (KOA) sufficient to selectively increase urine output, wherein the KOA decreases or does not change urinary excretion of electrolytes.

45. A method of treating a subject afflicted with a renal disease, the method comprising administering to the subject an amount of a kappa opioid receptor agonist (KOA) sufficient to selectively increase urine output, wherein the KOA decreases or does not change urinary excretion of electrolytes.

46. The method of claim 44 or claim 45, wherein treating comprises increasing urine output, decreasing urinary sodium and potassium excretion, decreasing urine osmolality, decreasing mean arterial pressure, or a combination thereof.

47. The method of claim 44, wherein the cardiovascular disease comprises hypertension, heart failure, or a combination thereof.

48. The method of claim 45, wherein the renal disease comprises acute or chronic kidney disease.

49. The method of claim 44, wherein the kappa opioid receptor agonist decreases mean arterial pressure (MAP).

50. The method of claim 44 or claim 45, further comprising administering the subject one or more additional active agents.

51. The method of claim 50, wherein the one or more additional active agents comprises a diuretic, an antihypertensive medication, or a combination thereof.

52. The method of claim 51, wherein the diuretic comprises a loop diuretic, a thiazide or thiazide-like diuretic, or a potassium sparing diuretic.

53. The method of claim 52, wherein the loop diuretic comprises furosemide, torsemide, ethacrynic acid, or bumetanide.

54. The method of claim 52, wherein the thiazide or thiazide-like diuretic comprises hydrochlorothiazide, chlorthalidone, metolazone, indapamide, chlorothiazide, or bendroflurnethiazide.

55. The method of claim 52, wherein the potassium sparing diuretic comprises amiloride, triamterene, spironolactone, or eplerenone.

56. The method of claim 51, wherein the antihypertensive medication comprises an angiotensin receptor blocking agent, an angiotensin converting enzyme inhibitor, a beta blocker, an alpha receptor blocker, a centrally acting sympatholytic, a calcium channel blocker, or a direct acting vascular smooth muscle dilator.

57. The method of claim 44 or claim 45, wherein the subject suffers from diuretic resistance.

58. The method of claim 57, wherein the subject has failed to achieve diuresis with diuretic therapy prior to administering.
